(19) United States
(12) Patent Application Publication
(10) Pub. No.: US 2026/0347440 A1
(43) Pub. Date: Nov. 5, 2026

(54) ANTIBODIES AND METHOD OF USE FOR P. GINGIVALIS

(71) Applicants: QJM BIOINFORMATION LTD., Orpington, Kent (GB); ZHENJIANG YANGTZE GREEN BIOTECHNOLOGY LTD., Zhanjiang, Jiangsu (CN)

(72) Inventors: Xiaoju SHI, Orpington, Kent (GB); Maria Xing XU, Manchester Greater Manchester (GB)

(73) Assignees: QJM BIOINFORMATION LTD., Orpington, Kent (GB); ZHENJIANG YANGTZE GREEN BIOTECHNOLOGY LTD., Zhenjiang, Jiangsu (CN)

(21) Appl. No.: 19/109,504

(22) PCT Filed: Sep. 16, 2022
(86) PCT No.: PCT/EP2022/075842

§ 371 Date: Mar. 6, 2025

(51) Int. Cl. C07K 16/1203; A61K 39/02; A61K 49/00; A61P 31/04; A61P 37/04; C07K 14/195; G01N 33/569

(52) U.S. Cl. CPC C07K 16/1203; A61K 39/0208; A61K 49/0008; A61P 31/04; A61P 37/04; C07K 14/195; G01N 33/56911; G01N 2333/195; G01N 2469/10; G01N 2800/26; G01N 2800/52

(57) ABSTRACT

The present disclosure provides monoclonal antibodies, and method of use thereof, for diagnosing and/or treating P. gingivalis infection and/or chronic diseases resulted from or associated with such infection. Specific antigen-antibody reaction regions on each P. gingivalis outer membrane protein are also identified and used for developing vaccines for prevention and treatment. The monoclonal antibodies of the present disclosure cross-react with different antigen regions of different P. gingivalis outer membrane protein. Animal models and diagnostic kits for serological evaluation and assessment of prognosis and treatment effectiveness after P. gingivalis infection are also provided.

### FIELD AND BACKGROUND

The present disclosure relates to the fields of immunology and biologics, particularly, multi-target monoclonal antibodies against P. gingivalis infection.

Periodontal disease is one of the most common oral infectious diseases in the world. The global annual cost of oral disease treatment is about 442 billion US dollars, of which periodontal disease accounts for 10.5-12%. In the United States of America (USA), 17 million people with periodontal disease seek medical care each year, costing more than $6 billion. The United Kingdom (UK) National Health System (NHS) estimates that England and Wales spend more than £500 million a year for periodontal care. These charges apply only to common acute periodontal treatment by NHS dentists and do not include specialized treatments, follow-up and hospital services, and private consultations. 80-97% of adults in China have periodontal problems of varying degrees.

Porphyromonas gingivalis (P. gingivalis) is recognized in the research field as an etiological agent of “adult” periodontal disease. Published literature suggests that periodontal disease in children with P. gingivalis infection is not as severe as adults. The survival rate of dental implants is directly related to the health of the oral periodontal tissue. P. gingivalis is the main pathogen causing peri-implantitis. In fact, most patients with periodontal disease have a chronic, progressive, recurring disease course, signifying that the bacterial infection has never been eradicated.

Many clinical and statistical research studies show that P. gingivalis not only causes oral and periodontal lesions, but also is related to cardiovascular disease, Alzheimer's disease, diabetes, and tumors of the digestive system and respiratory system. P. gingivalis infection is closely correlated to increased incidence of low-birth-weight babies and premature infants, and is strongly implicated in many chronic, systemic diseases. Up to date, the pathogenic mechanism associated with local infection and systemic disease is still unclear, and sometimes experimental results conflict with the proposed research direction.

P. gingivalis has many different subtypes, and these subtypes have different bacterial virulence. Bacterial virulence and abnormal immune responses have been the focus of debate in the scientific community when discussing bacterial pathogenic mechanisms.

At present, the main methods of clinical treatment of periodontal disease are the use of broad-spectrum antibiotics, physical/mechanical methods to remove local lesions, and surgical treatment if necessary. Ongoing areas of clinical practice that need resolution include how to prevent, treat and control P. gingivalis infection, reduce local and systemic damage caused by the bacterial infection, how to select biological products with immune protection, and how to evaluate animal experimental models for the function of new drugs.

### SUMMARY

The present disclosure provides safe, stable and effective monoclonal antibody drugs to prevent and treat diseases caused by P. gingivalis infection, including but not limited to periodontal disease.

The present disclosure provides that antibodies against P. gingivalis outer membrane protein are involved in local tissue and systemic damage occurs in infected individuals. The present disclosure further provides the heterophilic antigenic characteristics of P. gingivalis outer membrane proteins and the pathogenesis of local and systemic injury caused by P. gingivalis infection. Furthermore, the new animal model provided herein can be used to evaluate the protective function of the product.

In certain embodiments, the present disclosure provides the pathogenic mechanism of P. gingivalis, the specific monoclonal antibodies, and method of making thereof, against P. gingivalis. The specific antigen targets corresponding to the monoclonal antibodies, and method of identifying such antigen targets are also provided. In certain embodiments, novel tag-free RagB and Cra4S1 recombinant plasmids, and the nucleotide and amino acid sequences of the plasmids are shown in SEQ ID NOs: 1-10. Processes of producing recombinant proteins from plasmids, including the design of artificially synthesized genes, expression cassettes and recombinant vectors or cells are also provided. In certain embodiments, the specific antigen target sequences of the above recombinant proteins are shown in SEQ ID NOs: 11-126.

The monoclonal antibodies disclosed in the present disclosure are against multiple antigen targets disclosed herein. In certain embodiments, the nucleotide and amino acid sequences of the monoclonal antibodies corresponding to the antigen target are shown in SEQ ID NO. 128-167. In certain embodiments of the present disclosure, bispecific antibody and method of making thereof, are also provided. In certain embodiments, the nucleotide and amino acid sequences of the bispecific antibodies are shown in SEQ ID NO. 168-174. Methods of making such bispecific antibodies are also provided.

The present disclosure further provides a method of preventing and treating periodontal disease/peri-implantitis caused by chronic infection of P. gingivalis and other diseases related to chronic infection of P. gingivalis using one or more monoclonal antibodies disclosed herein. In certain embodiments, a combined antibody formula is that the mass ratio of RagB-4-1B11-4-7-7 monoclonal antibody to RagB-4-1C3-7-8 monoclonal antibody is 1:0.5-5. The present disclosure further provides applications of the monoclonal and/or bispecific antibody expression cassette, the recombinant vector, and the host cell in the preparation of medicines for preventing or treating periodontal disease/peri-implantitis and those with chronic diseases caused by P. gingivalis infections. The monoclonal and/or bispecific antibodies disclosed herein are used in prevention of primary disease, recurrence of disease in patients with previous periodontal disease/peri-implantitis, and those with chronic infectious diseases and non-oral/dental diseases caused by P. gingivalis infections.

The present disclosure further provides preventative and/or therapeutic vaccines for periodontal disease and/or peri-implantitis or diseases associated with P. gingivalis infection. In certain embodiments, the amino acid sequence of such vaccine is shown in SEQ ID NO. 127. The vaccines disclosed herein are used in prevention of primary disease, recurrence of disease in patients with previous periodontal disease/peri-implantitis, and those with chronic infectious diseases and non-oral/dental diseases caused by P. gingivalis infections.

The present disclosure also provides a novel animal model. In certain embodiment, an animal is infected by P. gingivalis and used for evaluating the monoclonal antibody of the present disclosure and/or antibody-antigen reaction or cross-reaction, and effects on animal's offspring.

The present disclosure further provides a serological diagnostic kit with specific diagnostic reagents for diagnosing and evaluating the treatment effect and prognosis of P. gingivalis infection-related diseases. In certain embodiments, the amino acid sequence of such diagnostic reagents is shown in SEQ ID NO. 2, 4, 6, 8 and 10. The diagnostic kit also provides an instruction on performing reaction using the reagent to detect specific antibodies from a biologic sample, which includes but is not limited to, a body fluid such as blood, gingival crevicular fluid, urine, saliva, cerebrospinal fluid, pleural and ascites fluid, and amniotic fluid.

### BRIEF DESCRIPTION OF THE DRAWINGS

Many aspects of the present disclosure can be better understood with reference to the drawings. The components in the drawings are not necessarily to scale, emphasis instead being placed upon clearly illustrating the principles of the present disclosure. Moreover, in the drawings, like reference numerals designate corresponding parts throughout the several views. A better understanding of the features and advantages of the disclosed invention will be obtained by reference to the following detailed description that sets forth illustrative embodiments, in which the principles of the invention are utilized, and the accompanying drawings (also “Figure” and “FIG.” herein), of which:

FIGS. 1A-1B show the nucleic acid and amino acid sequence for RagB-1 of P. gingivalis. FIG. 1A shows SEQ ID NO: 1 full length of 1461 base, C+G average (55.10%). FIG. 1B shows SEQ ID NO: 2 full length of 482 amino acid. The details of which are described in Example 1.

FIGS. 2A-2B show the nucleic acid and amino acid sequence for RagB-2 of P. gingivalis. FIG. 2A shows SEQ ID NO: 3 full length of 1449 base, C+G average (51.62%). FIG. 2B shows SEQ ID NO: 4 full length of 480 amino acid. The details of which are described in Example 1.

FIGS. 3A-3B show the nucleic acid and amino acid sequence for RagB-3 of P. gingivalis. FIG. 3A shows SEQ ID NO: 5 full length of 1470 base, C+G average (51.29%). FIG. 3B shows SEQ ID NO: 6 full length of 485 amino acid. The details of which are described in Example 1.

FIGS. 4A-4B show the nucleic acid and amino acid sequence for RagB-4 of P. gingivalis. FIG. 4A shows SEQ ID NO: 7 full length of 1455 base, C+G average (51.00%). FIG. 4B shows SEQ ID NO: 8 full length of 480 amino acid. The details of which are described in Example 1.

FIGS. 5A-5B show the nucleic acid and amino acid sequence for Cra4S1 of P. gingivalis. FIG. 5A shows SEQ ID NO: 9 full length of 411 base, C+G average (52.55%). FIG. 5B shows SEQ ID NO: 10 full length of 132 amino acid. The details of which are described in Example 1.

FIG. 6 shows that CLUSTALW (1.81) multiple sequence alignment (peptides) between four major outer membrane proteins, including RagB-1, RagB-2, RagB-3 and RagB-4. FIG. 6 discloses SEQ ID Nos: 2 (for RagB-1, W50 RagB), 4 (for RagB-2, ThaiRagB), 6 (for RagB-3, QMULRagB) and 8 (for RagB-4, 381 RagB). The details of which are described in Example 1.

FIGS. 7A-7B show the detection of recombinant protein expression and identification of purified recombinant protein RagB-1. FIG. 7A shows recombinant protein expression after induction, lane M: protein molecular weight scale (15-120 kDa); lane 1-3: BSA standard 0.5 μg, 1 μg, 2 μg; lane 4-6: total protein of bacteria (whole bacterial lysate) of seed cells, 7.4 hours culture and 24 hours culture; lane 7-9: supernatant after whole bacterial lysate centrifugation of seed cells, 7.4 hours culture and 24 hours culture; lane 10-12: precipitate after whole bacterial lysate centrifugation of seed cells, 7.4 hours culture and 24 hours culture. FIG. 7B, SDS-PAGE detection of purified recombinant protein, lane M: protein molecular weight scale (15-120 kDa); lane 1: non-reduction recombinant RagB-1; lane 2: reduction recombinant RagB-1. The details of which are described in Example 1.

FIGS. 8A-8B show the detection of recombinant protein expression and identification of purified recombinant protein RagB-2. FIG. 8A shows recombinant protein expression after induction, lane M: protein molecular weight scale (15-120 kDa); lane 1-3: BSA standard 0.5 μg, 1 μg, 2 μg; lane 4-6: total protein of bacteria (whole bacterial lysate) of seed cells, non-induction culture and 22 hours culture; lane 7-9: supernatant after whole bacterial lysate centrifugation of seed cells, non-induction culture and 22 hours culture. FIG. 8B shows SDS-PAGE detection of purified recombinant protein, lane M: protein molecular weight scale (15-120 kDa); lane 1: non-reduction recombinant RagB-2; lane 2: reduction recombinant RagB-2. The details of which are described in Example 1.

FIGS. 9A-9B show the detection of recombinant protein expression and identification of purified recombinant protein RagB-3. FIG. 9A shows recombinant protein expression after induction, lane M: protein molecular weight scale (15-120 kDa); lane 1-3: BSA standard 0.5 μg, 1 μg, 2 μg; lane 4-6: total protein of bacteria (whole bacterial lysate) of seed cells, non-induction cultures and 22 hours cultures; lane 7-9: supernatant after whole bacterial lysate centrifugation of seed cells, non-induction culture and 22 hours culture. FIG. 9B shows SDS-PAGE detection of purified recombinant protein, lane M: protein molecular weight scale (15-120 kDa); lane 1: non-reduction recombinant RagB-3; lane 2: reduction recombinant RagB-3. The details of which are described in Example 1.

FIGS. 10A-10B show the detection of recombinant protein expression and identification of purified recombinant protein RagB-4. FIG. 10A shows recombinant protein expression after induction, lane M: protein molecular weight scale (15-120 kDa); lane 1-3: BSA standard 0.5 μg, 1 μg, 2 μg; lane 4-6: total protein of bacteria (whole bacterial lysate) of seed cells, 6 hours culture and 15.5 hours culture; lane 7-9: supernatant after whole bacterial lysate centrifugation of seed cells, 6 hours culture and 15.5 hours culture; lane 10-12: precipitate after whole bacterial lysate centrifugation of seed cells, 6 hours culture and 15.5 hours culture. FIG. 10B shows SDS-PAGE detection of purified recombinant protein, lane M: protein molecular weight scale (15-120 kDa); lane 1: non-reduction recombinant RagB-4; lane 2: reduction recombinant RagB-4. The details of which are described in Example 1.

FIGS. 11A-11B show the detection of recombinant protein expression and identification of purified recombinant protein Cra4S1. FIG. 11A shows recombinant protein expression after induction, lane M: protein molecular weight scale (15-120 kDa); lane 1-3: BSA standard 0.5 μg, 1 μg, 2 μg; lane 4-6: total protein of bacteria (whole bacterial lysate) of seed cells, 14 hours culture and 23 hours culture; lane 7-9: supernatant after whole bacterial lysate centrifugation of seed cells, 14 hours culture and 23 hours culture; lane 10-12: precipitate after whole bacterial lysate centrifugation of seed cells, 14 hours culture and 23 hours culture. FIG. 11B shows the SDS-PAGE detection of purified recombinant protein, lane M: protein molecular weight scale (15-120 kDa); lane 1: non-reduction recombinant Cra4S1; lane 2: reduction recombinant Cra4S1. The details of which are described in Example 1.

FIG. 12A-12B show that the distribution of antibodies against different outer membrane proteins of P. gingivalis from healthy people, total of 51 clinical samples of serum. FIG. 12A shows the average OD value of the lowest quintile (10/51) of samples was set up as a negative value. The positive rate was set as OD>2.5 times of the negative value. The positive rate of RagB-1 antibody was 9.8% (5/51), high density antibody (set as OD>4 times of the negative value) was 2% (1/51); the positive rate of RagB-2 antibody was 0% (0/51); the positive rate of RagB-3 antibody was 0% (0/51), but high density antibody was 2% (1/51); 30 the positive rate of RagB-4 antibody was 17.6% (9/51), high density antibody was 13.7% (7/51); the positive rate of Cra4S1 antibody was 9.8% (5/51), high density antibody was 0% (0/51); the positive rate of unrelated protein GST antibody was 0% (0/51). FIG. 12B shows the proportion of antibody distribution in the analyzed sample. The details of which are described in Example 5.

FIG. 13 shows that the distribution of P. gingivalis ragB gene from 107 periodontal disease patients' clinical samples of gingival crevicular fluid. The results showed that 28 patients were negative for P. gingivalis 16s RNA (28/107, 26.2%) by PCR. Among 16s RNA positive samples, 7 patients were RagB-1 subtype of P. gingivalis positive by PCR ragB amplification (7/107, 6.6%), 30 patients were RagB-2 subtype positive (30/107, 28.0%), 32 patients were RagB-3 subtype positive (32/107, 29.9%), 6 patients were RagB-4 subtype positive (6/107, 5.6%), 4 patients were of unknown ragB subtype (4/107, 3.7%). The details of which are described in Example 5.

FIG. 14A-14B show the distribution of antibodies against different outer membrane proteins of P. gingivalis from geriatric patients, a total of 62 clinical samples of serum. FIG. 14A shows the average OD value of the lowest quintile (12/62) of samples was set up as a negative value. The positive rate was set as OD>2.5 times of the negative value. The he positive rate RagB-1 antibody was 25.8% (16/62), high density antibody (set as OD>4 times of the negative value) was 22.6% (14/62); the positive rate of RagB-2 antibody was 24.2% (15/62), high density antibody was 24.2% (15/62); the positive rate of RagB-3 antibody was 22.6% (14/62), high density antibody was 24.2% (15/62); the positive rate of RagB-4 antibody was 16.1% (10/62), high density antibody was 22.6% (14/62); the positive rate of Cra4S1 antibody was 22.6% (14/62), high density antibody was 21% (13/62); the positive rate of unrelated protein GST antibody was 19.4% (12/62), high density antibody was 21% (13/62). FIG. 14B shows the proportion of antibody distribution in the analyzed sample. The details of which are described in Example 5.

FIG. 15A-15B show the distribution of antibodies against different outer membrane proteins of P. gingivalis from cardiovascular patients, totaling 49 clinical samples of serum. FIG. 15A shows the average OD value of the lowest quintile (10/49) of samples was set up as a negative value. The positive rate was set as OD>2.5 times of the negative value. The positive rate of RagB-1 antibody was 36.7% (18/49), high density antibody (OD>4 times of negative value) was 8.2% (4/49); the positive rate of RagB-2 antibody was 32.7% (16/49), high density antibody was 8.2% (4/49); the positive rate of RagB-3 antibody was 38.8% (19/49), high density antibody was 8.2%(4/49); the positive rate of RagB-4 antibody was 46.9% (23/49), high density antibody was 10.2% (5/49); the positive rate of Cra4S1 antibody was 36.7% (18/49), high density antibody was 4.1% (2/49); the positive rate of unrelated protein GST antibody was 49% (24/49), high density antibody was 4.1% (2/49). FIG. 15B shows the proportion of antibody distribution in the analyzed sample. The details of which are described in Example 5.

FIG. 16 shows that protein sequence alignments between RagB-4 of P. gingivalis and outer membrane protein of Porphyromonas gulae strain COT-052 OH2179. The Query sequence is P. gulae outer membrane protein sequence (PubMed ID: 25858832, GenBank: JRAJ01000005.1), subject sequence is RagB-4 (SEQ ID NO: 8). Two proteins have a 93% identity similarity rate. The details of which are described in Example 5.

FIGS. 17A-17E show the study of passive immune function using the combination of two targeted specific antibodies. Pictures were taken on day 12 post challenge of P. gingivalis. FIG. 17A shows group G1 mice received monoclonal antibody (MAb) 1D2-2-1-3. FIG. 17B shows group G2 mice received MAb 1D2-2-1-3 and mouse polyclonal antibody against Cra4S1. FIG. 17C shows group G3 mice received mouse polyclonal antibody against Cra4S1. FIG. 17D shows group G4 mice received normal mouse serum. FIG. 17E shows group G5 mice received PBS as a challenge control. The details of which are described in Example 6.

FIGS. 18A-18B shows the passive immune protective function of combined antibodies, the details of which are described in Example 6, the X-coordinate being the groups of tested animals, and the Y-coordinate being the area of skin and soft tissue lesions (mm²). FIG. 18A shows the results of day 5 post challenge. FIG. 18B shows the results of day 12 post challenge. The details of which are described in Example 6. One-way ANOVA analysis was used, *P<0.05.

FIGS. 19A-19D show the monoclonal antibody RagB-2-1A4-3-7-7 sequences. FIG. 19A shows heavy chain (Mouse IgG1) variable region (V_{H}) DNA sequence confirmed by sequencing (SEQ ID NO: 128). FIG. 19B shows V_{H} Amino Acid Sequence (SEQ ID NO: 129). FIG. 19C shows light chain (Mouse Kappa) variable region (V_{L}) DNA sequence confirmed by sequencing (SEQ ID NO: 133). FIG. 19D shows V_{L} amino acid sequence (SEQ ID NO: 134), the details of which are described in Example 7.

FIGS. 20A-20F show the monoclonal antibody RagB-3-1D2-2-1-3 sequences and the detection of purified recombinant antibody which is expressed in eukaryotic HEK293 cells and purified. FIG. 20A shows heavy chain (Mouse IgG2a) variable region (V_{H}) DNA sequence confirmed by sequencing (SEQ ID NO: 138). FIG. 20B shows V_{H} Amino Acid Sequence (SEQ ID NO: 139). FIG. 20C shows light chain (Mouse Kappa) variable region (V_{L}) DNA sequence confirmed by sequencing (SEQ ID NO: 143). FIG. 20D shows V_{L} amino acid sequence (SEQ ID NO: 144). FIG. 20E shows recombinant antibody purification in HPLC, 280 nm collection peak. FIG. 20F shows confirmation of purified recombinant antibody on SDS-PAGE, lane M: protein molecular weight scale (15-120 kDa); lane 1: purified RagB-3-1D2-2-1-3. The details of which are described in Example 7.

FIGS. 21A-21B show the monoclonal antibody RagB-4-1B11-4-4 sequences and the detection of purified recombinant antibody RagB-4-1B11-4-4 which is expressed in eukaryotic HEK293 cells and purified. FIG. 21A shows heavy chain (Mouse IgG1) variable region (V_{H}) DNA sequence confirmed by sequencing (SEQ ID NO: 148). FIG. 21B shows V_{H} Amino Acid Sequence (SEQ ID NO: 149). FIG. 21C shows light chain (Mouse Kappa) variable region (V_{L}) DNA sequence confirmed by sequencing (SEQ ID NO: 153). FIG. 21D shows V_{L} amino acid sequence (SEQ ID NO: 154). 21E shows recombinant antibody purification in HPLC, 280 nm collection peak. FIG. 21F shows confirmation of purified recombinant antibody on SDS-PAGE, lane M: protein molecular weight scale (15-120 kDa); lane 1: purified RagB-4-1B11-4-4. The details of which are described in Example 7.

FIGS. 22A-22B show the monoclonal antibody RagB-4-1C3-7-8 sequences and the detection of purified recombinant antibody RagB-4-1C3-7-8 which is expressed in eukaryotic HEK293 cells and purified. FIG. 22A shows heavy chain (Mouse IgG1) variable region (V_{H}) DNA sequence confirmed by sequencing (SEQ ID NO: 158). FIG. 22B shows V_{H} Amino Acid Sequence (SEQ ID NO: 159). FIG. 22C shows light chain (Mouse Kappa) variable region (V_{L}) DNA sequence confirmed by sequencing (SEQ ID NO: 163). FIG. 22D shows V_{L} amino acid sequence (SEQ ID NO: 164). FIG. 22E shows recombinant antibody purification in HPLC, 280 nm collection peak. FIG. 22F shows confirmation of purified recombinant antibody on SDS-PAGE, lane M: protein molecular weight scale (15-120 kDa); lane 1: RagB-4-1C3-7-8. The details of which are described in Example 7.

FIGS. 23A-23H show the designed sequences of bispecific antibody 1B11-1C3, and detection of recombinant plasmid expressed in eukaryotic HEK293 cells. FIG. 23A shows recombinant heavy chain variable region (V_{H}) DNA sequence confirmed by sequencing (SEQ ID NO: 168). FIG. 23B shows V_{H} Amino Acid Sequence (SEQ ID NO: 169). FIG. 23C shows sequence diagram of the bispecific antibody heavy chain, bold and underlined indicate the original specific MAb. FIG. 23D shows light chain (Mouse Kappa) variable region (V_{L}) DNA sequence confirmed by sequencing (SEQ ID NO: 173). FIG. 23E shows V_{L} amino acid sequence (SEQ ID NO: 174). FIG. 23F shows sequence diagram of the bispecific antibody light chain, bold and underlined indicate the original specific MAb. FIG. 23G shows recombinant bispecific antibody purification in HPLC, 280 nm collection peak. FIG. 23H shows confirmation of purified recombinant antibody on SDS-PAGE, lane M: protein molecular weight scale (15-120 kDa); lane R: reduction 1B11-1C3, 76 kDa (heavy chain)/24 kDa (light chain), lane N-R: non-reduction 1B11-1C3, 200 kDa. The details of which are described in Example 8.

### DETAILED DESCRIPTIONS

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### DEFINITIONS

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a therapeutic agent” including, but not limited to, one or more such therapeutic agents and/or combinations of one or more therapeutic agents, and the like.

Reference to “a/an” chemical compound, therapeutic agent, and pharmaceutical composition each refers to one or more molecules of the chemical compound, therapeutic agent, and pharmaceutical composition rather than being limited to a chemical compound, therapeutic agent, and pharmaceutical composition, the one or more molecules may or may not be identical, so long as they fall under the category of the chemical compound, therapeutic agent, and pharmaceutical composition. Thus, for example, “a” therapeutic agent is interpreted to include one or more molecules of the therapeutic agent, where the therapeutic agent molecules may or may not be identical (e.g., comprising different isotope abundances and/or different degrees of hydration or in equilibrium with different conjugate base or conjugate acid forms).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of inflammation associated with any disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e., one atmosphere).

Treatment and/or Prevention Method

Disclosed herein is a method of treating and/or preventing P. gingivalis infection in various diseases and/or associated pathological conditions in a patient in need thereof, comprising administering to the patient a therapeutically effective amount of one or more monoclonal and/or bispecific antibodies disclosed herein. In certain embodiments, provided herein is a method for reducing the level of infections caused by P. gingivalis. Methods for measuring the extent of infection are well known in the art. In one embodiment, the level of infection is reduced by about 5% to about 100%. In one embodiment, the level of infection is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

### Monoclonal and Bispecific Antibodies

As disclosed herein, in certain embodiments, the nucleotide and amino acid sequences of certain idiotypic antibodies are provided as follows:
1. RagB-2-1A4-3-7-7 monoclonal antibody comprises a heavy chain (Mouse IgG1) variable region (V_{H}) comprising a nucleic acid sequence (357 base) as set forth in SEQ ID NO:128 which encodes an amino acid sequence (119 aa) as set forth in SEQ ID NO:129; and a light chain (Mouse Kappa) variable region (V_{L}) comprising a nucleic acid sequence (282 base) as set forth in SEQ ID NO: 133 which encodes an amino acid sequence (94 aa) as set forth in SEQ ID NO: 134.
2. RagB-3-1D2-2-1-3 monoclonal antibody comprises a heavy chain (Mouse IgG2a) variable region (V_{H}) comprising a nucleic acid sequence (360 base) as set forth in SEQ ID NO:138 which encodes an amino acid sequence (120 aa) as set forth in SEQ ID NO:139; and a light chain (Mouse Kappa) variable region (V_{L}) comprising a nucleic acid sequence (336 base) as set forth in SEQ ID NO: 143 which encodes an amino acid sequence (112 aa) as set forth in SEQ ID NO: 144
3. RagB-4-1B11-4-4 monoclonal antibody comprises a heavy chain (Mouse IgG1) variable region (V_{H}) comprising a nucleic acid sequence (375 base) as set forth in SEQ ID NO:148 which encodes an amino acid sequence (125 aa) as set forth in SEQ ID NO:149; and a light chain (Mouse Kappa) variable region (V_{L}) comprising a nucleic acid sequence (336 base) as set forth in SEQ ID NO: 153 which encodes an amino acid sequence (112 aa) as set forth in SEQ ID NO: 154.
4. RagB-4-1C3-7-8 monoclonal antibody comprises a heavy chain (Mouse IgG1) variable region (V_{H}) comprising a nucleic acid sequence (357 base) as set forth in SEQ ID NO:158 which encodes an amino acid sequence (119 aa) as set forth in SEQ ID NO:159, and a light chain (Mouse Kappa) variable region (V_{L}) comprising a nucleic acid sequence (321 bases) as set forth in SEQ ID NO:163 which encodes an amino acid sequence (107 aa) as set forth in SEQ ID NO:164.
5. 1B11-1C3 bispecific antibody comprises a heavy chain (Mouse IgG1) variable region (V_{H}) comprising a nucleic acid sequence (2232 bases) as set forth in SEQ ID NO:168 which encodes an amino acid sequence (728 aa) as set forth in SEQ ID NO:172, and a light chain (Mouse Kappa) variable region (V_{L}) comprising a nucleic acid sequence (744 bases) as set for the in SEQ ID NO:173 which encodes an amino acid sequence (232 aa) as set forth in SEQ ID NO: 174.

### Dosing and Administration

While it is possible for an active ingredient to be administered alone, it may be preferable to present them as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carriers must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10. The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

### Pharmaceutical Compositions

The pharmaceutical compositions of the disclosure provide for an effective amount of one or more antibodies disclosed herein.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In certain embodiments, the antibodies of the present disclosure may be formulated in any suitable dosage form for an appropriate administration. In certain embodiments, the methods provided herein comprise administering a pharmaceutical composition comprising one or more antibodies of the present disclosure and a pharmaceutically acceptable carrier or excipient. Combination formulations and/or treatment according to the present disclosure comprise the antibody of the present disclosure together with one or more pharmaceutically acceptable carriers or excipients and optionally other therapeutic agents, now known or later developed, for treating and/or preventing infections. Combination formulations containing the active ingredient may be in any form suitable for the intended method of administration.

In certain embodiments, the vaccine of the present disclosure is formulated in any suitable dosage form for an appropriate administration. In certain embodiments, the methods provided herein comprise administering a pharmaceutical composition comprising one or more vaccines of the present disclosure and a pharmaceutically acceptable carrier or excipient. Combination formulations and/or treatment according to the present disclosure comprise the vaccine of the present disclosure together with one or more pharmaceutically acceptable carriers or excipients and optionally other therapeutic agents, now known or later developed, for treating and/or preventing infections. Combination formulations containing the active ingredient may be in any form suitable for the intended method of administration.

In view of the technologies disclosed in PCT/GB2005/001976 and PCT/CN2019/124433, the rag locus encoding the bacterial outer membrane protein of P. gingivalis is identified by ragA and ragB which are two co-transcribed and independent genes. The data have shown that the outer membrane protein encoded by the ragB gene has four main different subtypes. These four subtypes account for more than 95% of 197 clinical sample collections of patients with periodontal disease from a worldwide collaboration. The four subtypes of P gingivalis, according to their RagB outer membrane proteins, were named W50ragB protein, ThairagB protein, QMLragB protein and 381 ragB protein, respectively.

There are significant differences in protein sequences among the four isoforms, with a similarity rate of only 42-59% in pair alignment (FIG. 6). It was found that RagA, like RagB, exists in several different isoforms. However, Cra4S1 is a polypeptide fragment near the N-terminus of the RagA protein encoded by the ragA gene, and is a conserved domain, meaning that the Cra4S1 polypeptide occurs in all various isoforms of P. gingivalis.

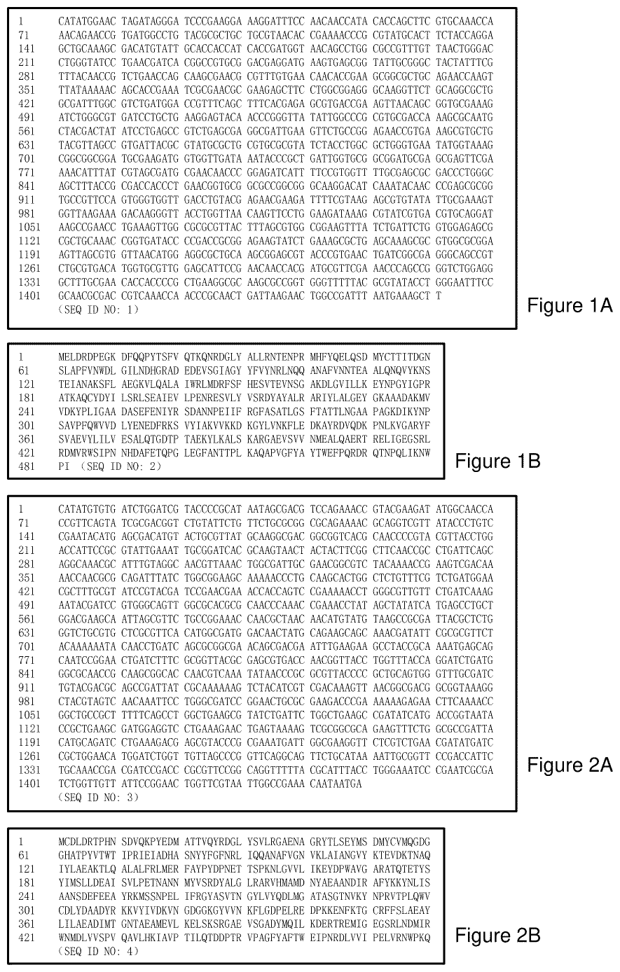

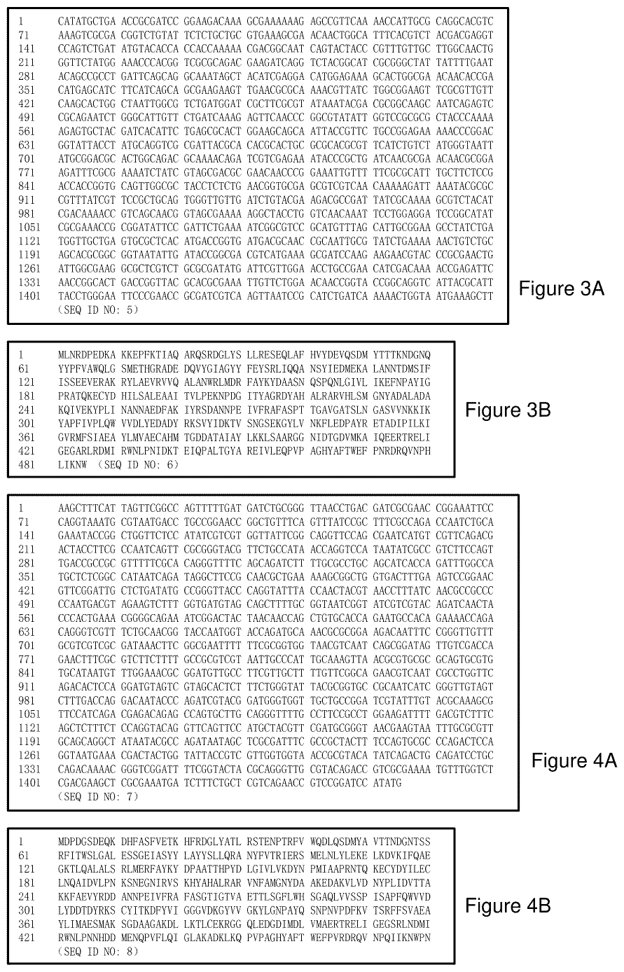

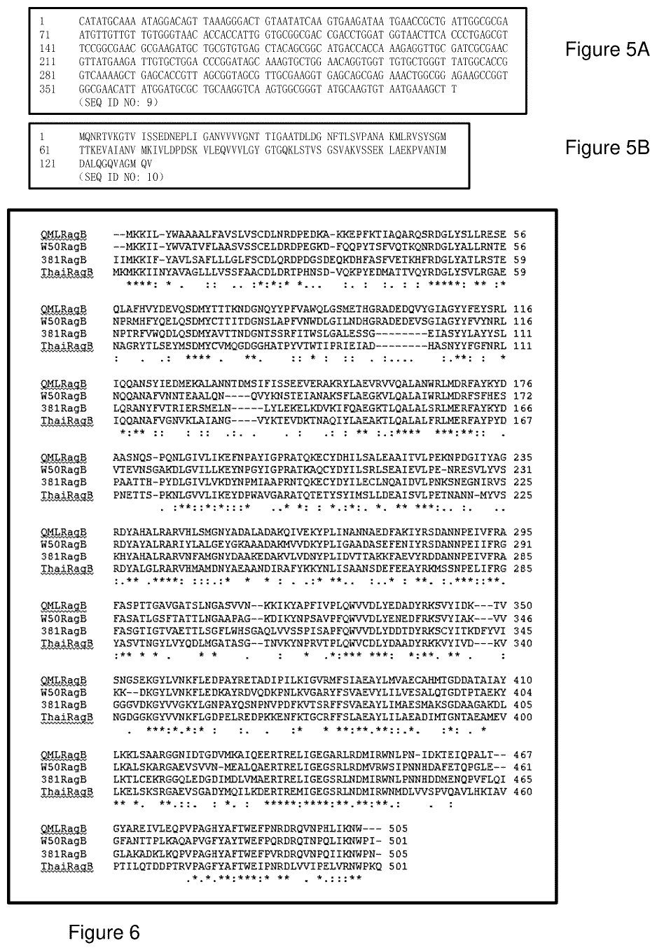

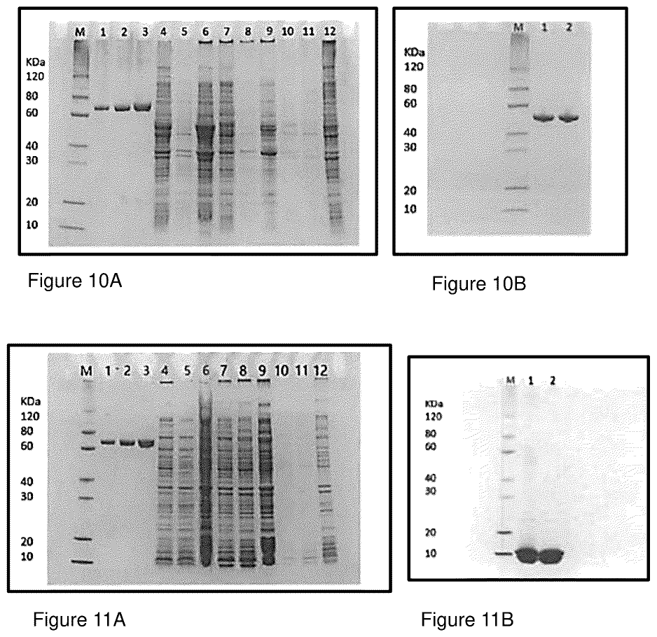

In certain embodiments, the present disclosure provides the gene modification sequence of P. gingivalis outer membrane protein RagB, and a successful completion of the pilot-scale production of tag-free RagB and Cra4S1 recombinant proteins (Example 1, FIGS. 1-5 and FIGS. 7-11).

The different RagB outer membrane protein subtypes each have their own antigenic characteristics. However, polyclonal antibody sera produced from animals immunized with a single recombinant RagB protein were found to have visible cross-reactivity with other RagB protein isoforms at certain dilutions (Example 2).

Mouse monoclonal antibodies against four different RagB and Cra4S1 proteins have been prepared, some of which not only react with homologous proteins but also cross-react with proteins of other subtypes (Example 3).

Although the sequence similarity of RagB isoforms is as low as 42-59%, cross-reactivity between different RagB proteins suggests the presence of common antigens or compatible epitopes. Are these cross-reactions caused by linear protein sequences, two-dimensional planar and/or multi-dimensional structures? What is the molecular basis of these cross-reactive targets? Where is the target antigen? Do monoclonal antibodies have cross-immune protection?

In certain embodiments, the present disclosure provides an artificially synthesized peptide library. The five peptide library sequences of P. gingivalis bacteria contain the peptide fragments of the different subtype of outer membrane protein RagB, and Cra4S1. These are used to screen the antigen targets of monoclonal antibodies. Peptide libraries of RagB and Cra4S1 proteins were then created, and monoclonal antibodies had the opportunity to react with peptide fragments at different positions throughout the corresponding protein to find binding targets. Several monoclonal antibodies with cross-reactive characteristics found corresponding targets in the peptide library. This important discovery has the potential to save both production processes and production costs. The previous approach was to target homologous bacteria with specific monoclonal antibodies depending on the strain of P. gingivalis, but this new approach is to use antibody products which can target all four subtypes of P. gingivalis simultaneously (Example 4).

More specifically, the present disclosure provides a pathogenic mechanism of P. gingivalis. In certain embodiments, the heterophilic antigen and individual heterophilic antibodies of P. gingivalis were identified as causing chronic persistence of the disease and damage to multiple tissues and organs, the methods and results of pathogenic mechanism of P. gingivalis.

In certain embodiments, the results of serological studies of clinical specimens showed that P. gingivalis outer membrane proteins including RagB and Cra4S1 are characteristic of heterophilic antigens. The results demonstrated that local lesions and systemic damage caused by P. gingivalis infection are closely related to heterophilic antigens and/or heterophilic antibodies (Example 5).

In certain embodiments, the present disclosure also provides an animal model for P. gingivalis infection. The animal model disclosed herein is used to evaluate the systemic damage caused by pathogenic bacteria infection, as well as a treatment efficacy for P. gingivalis infection.

The effect of the combined antibody on the mouse reproductive model has yielded profound results, which have wide ranging implications for the development of products for both prevention and treatment of P gingivalis and related chronic system diseases. Particularly important is the discovery of two monoclonal antibodies with adjacent antigen targets, which are directed against different subtypes of bacteria, and the positions and sequences of antigen targets in each outer membrane protein are identified. As research in this area expands, further indications for treatment are identified, and the antibodies and method of use thereof, disclosed herein open a broad prospective market for products used for prevention and/or treatment of various diseases beyond periodontal disease. Therefore, the present disclosure provides stable and efficient biological products which solve the problem of treating different subtypes of pathogenic bacteria. This simplifies the production process, reduces production costs, and results in an overall benefit to public health (Example 6).

In certain embodiments, the present disclosure provides the nucleotide and protein sequences of four monoclonal antibody genes produced by mice immunized with P. gingivalis outer membrane protein RagB. The recombinant antibody plasmids constructed with these antibody sequences were successfully expressed in eukaryotic cells. These retain the ability of the original monoclonal antibody to bind to the antigen, providing the molecular basis for therapeutic drug development and industrial production of the product.

The present disclosure further provides a humanized mutation scheme of one or more monoclonal antibodies and a large-scale production process for such antibodies disclosed herein (Example 7).

In certain embodiments, the present disclosure provides two specific monoclonal antibodies, whose antigen targets are closely connected and belong to the same subtype of P. gingivalis. Importantly, both antibodies are characterized by cross-reactivity with different isoforms of outer membrane proteins, and the peptide sequences and positions on outer membrane proteins of these cross-antigens are different. This discovery contributes greatly to simplifying the production process and effectively producing the products.

In certain embodiment, the present disclosure provides the design and operation of bispecific antibody against different subtypes of P. gingivalis, and the recombinant antibody plasmid constructed by using the designed antibody sequences has been successfully expressed in eukaryotic cells. The data presented herein show that the recombinant bispecific antibody not only retains the antigen-binding ability of the original different monoclonal antibodies, but also supplements the deficiencies of the original monoclonal antibodies. These data establish feasibility and promise in bispecific antibody product development (Example 8).

In certain embodiments, the present disclosure also provides a diagnostic kit for the diagnosis, treatment and prognostic assessment of diseases associated with P. gingivalis infection, including but not limited to periodontal disease, cardiovascular disease, rheumatoid arthritis, oral gastrointestinal cancer, ulcerative colitis, neurological disorders, autoimmune encephalomyelitis, lung cancer, adverse pregnancy, pancreatic cancer, diabetes, chronic kidney disease, bacterial pneumonia and chronic obstructive pulmonary disease.

The present disclosure provides reagents of diagnostic kit for the diagnosis, treatment and prognostic assessment of diseases associated with P. gingivalis infection. In certain embodiments, the amino acid sequence of such diagnostic reagents is shown in SEQ ID NO. 2, 4, 6, 8 and 10. The diagnostic kit also provides an instruction on performing reaction using the reagent to detect specific antibodies from a biologic sample, which includes but is not limited to, a body fluid such as blood, gingival crevicular fluid, urine, saliva, cerebrospinal fluid, pleural and ascites fluid, and amniotic fluid. The methods, making, using and results analysis of the diagnostic kit are detailed in Example 9 of the present disclosure.

The present invention also discloses a polypeptide vaccine that mobilizes the active immune function of a subject of interest. In certain embodiments, the sequence of the polypeptide vaccine is identified according to the target of a neutralizing antibody, the antigenic characteristics of the polypeptide vaccine is retained, and irrelevant and harmful components are removed. The polypeptide vaccine disclosed herein reduces common side effects and repeated administrations, providing cost-effective and long-lasting protection against P. gingivalis infection.

The present disclosure further provides polypeptide vaccines and combined antibodies used in active immunisation and passive immunisation on infected animals. The methods and results of making and using these polypeptide vaccines and/or combined antibodies are detailed in Example 10.

Now having described the aspects of the present disclosure, in general, the following Examples describe some additional and/or more detailed aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Example 1

### Expression of RagB and Cra4S1 in E. coli

The P. gingivalis outer membrane protein RagB has four major allelic isoforms that predominate in clinical periodontal specimens. The four different isoforms of outer membrane proteins are W50RagB, ThaiRagB, QMLRagB and 381 RagB.

In this disclosure, the names of the four RagB subtypes have been simplified, with RagB-1 replacing W50RagB, RagB-2 replacing ThaiRagB, RagB-3 replacing QMLRagB, and RagB-4 replacing 381 RagB. As shown in FIG. 6 and Table 1, the similarity of protein sequences between RagB alleles is only 42-57%, RagB-1 and RagB-2 share 47% of their sequence, RagB-1 and RagB-3 have 57% similarity, it is 49% for RagB-1 and RagB-4, RagB-2 and RagB-3 share 45% of their sequence, 42% for RagB-2 and RagB-4, and 49% for RagB-3 and RagB-4.

The above-mentioned four different P. gingivalis outer membrane proteins (RagBs) and Cra4S1 protein encoding genes, and recombinant plasmids constructed by the vector, were transfected into the host Escherichia coli (E. coli). All of them were successfully expressed after induction.

TABLE 1
RagB Multiple Sequence Alignments
(percentage of identical sequence)

| | RagB-1 | RagB-2 | RagB-3 | RagB-4 |
|---|---|---|---|---|
| RagB-1 | 100% | 47% | 57% | 49% |
| RagB-2 | 47% | 100% | 45% | 42% |
| RagB-3 | 57% | 45% | 100% | 49% |
| RagB-4 | 49% | 42% | 49% | 100% |

The original nucleotide sequence of the target protein was modified according to the codon preference of the host E. coli. After the nucleotide sequence was optimized, the encoded protein sequence remains the same as the original target protein, so that the target protein can be accurately expressed.

Specifically, the artificially synthesized codon-optimized nucleic acid sequences and amino acid sequences of RagB-1, RagB-2, RagB-3, RagB-4, and Cra4S1 are set forth in SEQ ID NO: 1-10, as well as in FIGS. 1A-1B (for RagB-1, SEQ ID NO:1-2), 2A-2B (for RagB-2, SEQ ID NO:3-4), 3A-3B (for RagB-3, SEQ ID NO:5-6), 4A-4B (for RagB-4, SEQ ID NO:7-8), and 5A-5B (for Cra4S1, SEQ ID NO:9-10).

TABLE 2
Recombinant Outer Membrane Protein Expressed in E. coli

| Recombinant Protein Name | RagB-1 | RagB-2 | RagB-3 | RagB-4 | Cra4S1 |
|---|---|---|---|---|---|
| Gene Length (bp) | 1461 | 1455 | 1470 | 1455 | 411 |
| Cloning Strategy | Ndel-HindIII | Ndel-HindIII | Ndel-HindIII | Ndel-HindIII | Ndel-HindIII |
| Resistant Antibiotic | Ampicillin/Kanamycin | Kanamycin | Kanamycin | Kanamycin | Ampicillin Kanamycin |
| Expected MW (kDa) | ~54.40 | ~55 | ~55 | ~55 | ~13.67 |
| CRO | GenScrpt, USA | DetaBio, China | DetaBio, China | DetaBio, China | GenScrpt, USA |
| Contract Number | C7997FI160 | DT1872 | DT1868 | DT1780 | C0904FK060 |

Five P. gingivalis outer membrane proteins including RagB-1 (W50 RagB), RagB-2 (ThaiRagB), RagB-3 (QMLRagB), RagB-4 (381 RagB), and Cra4S1 were all expressed in the host E. coli. Briefly described as follows, after the plasmid was successfully constructed, the cloned gene (containing optimized nucleotide sequence) was confirmed by sequencing to ensure the accuracy of the protein sequence. After the plasmid was transformed into E. coli, the expression of the recombinant protein was promoted by induction. The expressed bacteria were centrifuged to collect the precipitate, the bacterial sludge was mechanically broken, and the supernatant was purified by ion exchange chromatography to obtain the target protein. All recombinant proteins are tag-free and soluble (Table 2).

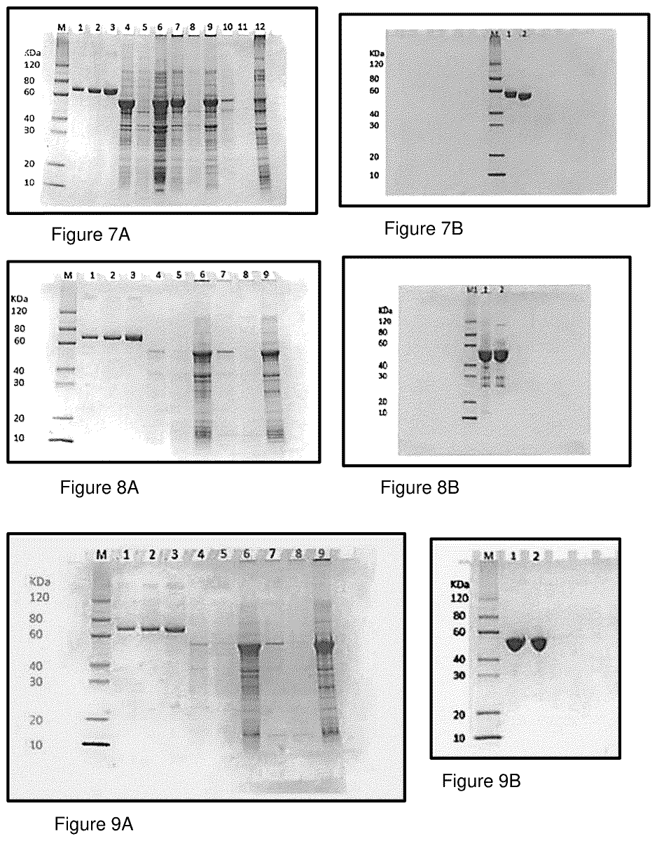

The pilot-scale production of the above five tag-free recombinant proteins was conducted by a third-party contract research organization (CRO) company (GenScript, USA, contract number C9458FG140). Specifically, the recombinant plasmid and/or seed bacteria were sent to the CRO, which took the next steps of fermentation production after confirmation and identification. During high-density fermentation, the medium was free of animal-derived proteins and antibiotics, and after fermentation was complete, the protein was purified by five-step chromatography. The five recombinant protein products were accompanied by a certificate of analysis. FIGS. 7A-7B (for RagB-1), 8A-8B (for RagB-2), 9A-9B (for RagB-3), 10A-10B (for RagB-4), and 11A-11B (for Cra4S1) show the induced expression and purification results of five tag-free target proteins.

TABLE 3
Pilot-scale production of recombinant protein

| Plasmid Name | Contract Number | Production batch | MW (kDa) | Concentration | Purity | Endotoxin detection | Certificate of Analysis |
|---|---|---|---|---|---|---|---|
| Cra4S1 | C9458FG140-1 | Cra4S1F210321FE | 13 KDa | 6.0 mg/ml | 98% | N/A | Yes |
| RagB-1 | C9458FG140-2 | RagB-1F201020F101 | 54.1 KDa | 3.1 mg/ml | 98% | <1 EU/mg | Yes |
| RagB-2 | C9458FG140-3 | RagB-2F200815F1-01-01 | 51.5 KDa | 3.4 mg/ml | 93% | <1 EU/mg | Yes |
| RagB-3 | C9458FG140-4 | RagB-3F200815F2-01 | 51.3 KDa | 10.7 mg/ml | 96% | <1 EU/mg | Yes |
| RagB-4 | C9458FG140-5 | RagB-4F201103F2 | 49.8 KDa | 1.0 mg/ml | 99% | <1 EU/mg | Yes |

Therefore, the successful pilot production of five P. gingivalis outer membrane proteins marks the feasibility of product industrialization.

### Example 2

### Cross-Reactivity of Polyclonal Antibodies Against P. Gingivalis Outer Membrane Protein

Although the RagB protein sequences of different subtypes of P. gingivalis are different, in the animal serological studies, the degree of antibody cross-reactivity to the subtypes had been measured. Thus, the cross-reaction index of antibodies to different RagB subtypes was added into the experimental design. This aspect of study mainly utilized the Enzyme-Linked Immunosorbent Assay (ELISA) method to detect antibodies with differing degrees of binding affinity.

Specifically, the preparation methods of mouse and rabbit antibody sera are briefly described as follows: after immunizing mice and rabbits with antigenic protein (RagB/Cra4S1) three or four times according to their antibody response, the titer of specific antibodies in the serum was determined by the indirect ELISA method. Once the antibody level reached a set standard, a sample of whole blood was collected from the animal, the serum was obtained after incubation and centrifugation, and was frozen for storage.

ELISA is an enzyme-linked immunosorbent assay. The basic principle is to fix a certain concentration of antigen on the surface of polystyrene microplate by physical adsorption, add the diluted serum (containing the primary antibody) to the reaction plate, allow time for the antigen and antibody to combine, and then wash away the free, unbound molecules. Next, add the enzyme-linked secondary antibody (anti-primary antibody), then again wash away the free molecules. Finally, add the enzyme substrate solution to allow a period of enzymatic reaction, and measure the color reaction of the substrate. The degree of color reaction indirectly reflects the qualitative and semi-quantitative characteristics of the substrate. Specific steps are as follows:
1. Coating antigen: proteins (antigen) solution prepared in PBS, concentration 4 μg/ml, add 100 μl of antigen solution to each well, seal the plate, and incubate overnight at room temperature (about 20 °C), wash the plate 4 times with PBS buffer, 300 μl/well, pat dry the residual buffer in the well on absorbent paper, add 100 μl blocking buffer (1% BSA in PBS) to each well and incubate at room temperature (20 °C) for 2 hours, wash the plate 4 times with PBS buffer, 300 μl/well, pat dry the residual buffer in the well on absorbent paper, the coated ELISA plate is stored in a low temperature freezer at −20 °C for 4-6 weeks.
2. Adding primary antibody: the cell culture supernatant or serum antibody is made into the required working concentration with sample dilution buffer (1% BSA in PBS), add 100 μl primary antibody solution to each well, seal the plate, and incubate at 37 °C for 60-90 minutes, wash the plate 4 times with a PBS buffer, 300 μl/well, and pat dry the residual buffer in the well on absorbent paper.
3. Adding enzyme-conjugated secondary antibody: according to the manufacturer's instructions, dilute the secondary antibody with a sample dilution buffer, add 100 μl enzyme-conjugated secondary antibody to each well, incubate at 37 °C for 40-60 minutes, wash the plate 3 times with PBS buffer, 300 μl/well, pat dry the residual buffer in the well on absorbent paper, lastly wash the plate with deionized water, 300 μl/well, and pat dry the residual liquid in the well on absorbent paper.
4. Reaction showing color: prepare the substrate solution; 4-nitrophenyl phosphate disodium salt hexahydrate is dissolved in 1M Tris-MgCl₂, 1 mg/ml, add 100 μl of substrate solution to each well and incubate at room temperature for 50-180 minutes in the dark, set the dual-wavelength (405 nm and 630 nm) program on the microplate reader, place the microplate in the preheated reader for reading, save the data, and analyze.

In the traditional ELISA method, there are many different available detection systems to choose from. In this Example, the alkaline phosphatase-conjugated secondary antibody was chosen for enzyme binding due to its long stability and low false-positive error. Specifically, for the ELISA plate No. 17003 shown in Table 4, the antigen layout described here is designed as follows: the 1st and 7th columns were coated with RagB-1 recombinant protein, the 2nd and 8th columns were coated with RagB-2, and the 3rd and 9th columns were coated RagB-3, the 4th and 10th columns were coated with RagB-4, the 5th and 11th columns were coated with Cra4S1 recombinant protein. The 6th and 12th columns were coated with GST recombinant protein as negative irrelevant protein controls. The serum of the test and the control group was diluted 1:2000, and each serum sample had the opportunity to detect 6-well antigens, including RagB-1, RagB-2, RagB-3, RagB-4, Cra4S1 and GST. The optical density (OD) values of No. 17003 ELISA plate are shown in Table 4, and PBS and normal serum were used as negative controls.

TABLE 4
P. gingivalis Infected Animal's Polyclonal Antibody Detection

| 17003 | RagB-1 | RagB-2 | RagB-3 | RagB-4 | Cra4S1 | GST | RagB-1 | RagB-2 | RagB-3 | RagB-4 | Cra4S1 | GST |
|---|---|---|---|---|---|---|---|---|---|---|---|---|
| Serial | 1 | 2 | 3 | 4 | 5 | 6 | 7 | 8 | 9 | 10 | 11 | 12 |
| A | 0.062 | 0.071 | 0.081 | 0.036 | 0.042 | 0.034 | 0.082 | 2.177 | 0.357 | 0.509 | 0.155 | 0.058 |
| B | 0.076 | 0.195 | 0.074 | 0.074 | 0.026 | 0.041 | 0.058 | 0.082 | 0.072 | 0.062 | 0.062 | 0.047 |
| C | 0.08 | 0.271 | 0.078 | 0.055 | 0.042 | 0.046 | 0.046 | 0.05 | 0.048 | 0.035 | 0.04 | 0.033 |
| D | 0.103 | 1.858 | 0.904 | 0.523 | 0.157 | 0.062 | 0.057 | 0.151 | 0.09 | 0.049 | 0.045 | 0.044 |
| E | 0.12 | 2.365 | 0.756 | 1.303 | 0.152 | 0.061 | 0.062 | 0.141 | 0.093 | 0.057 | 0.053 | 0.045 |
| F | 0.076 | 0.192 | 0.069 | 0.093 | 0.034 | 0.034 | — | — | — | — | — | — |
| G | 0.074 | 0.324 | 0.069 | 0.038 | 0.044 | 0.032 | — | — | — | — | — | — |
| H | 0.117 | 1.655 | 0.288 | 0.442 | 0.149 | 0.052 | 0.036 | 0.042 | 0.039 | 0.029 | 0.027 | 0.027 |
| lay out | 1 | 2 | 3 | 4 | 5 | 6 | 7 | 8 | 9 | 10 | 11 | 12 |

| | | |
|---|---|---|
| A | PBS | ♂G7-4 |
| B | ♂G6-1 | ♂G8-1 |
| C | ♂G6-2 | ♂G8-2 |
| D | ♂G6-3 | ♂G8-3 |
| E | ♂G6-4 | ♂G8-4 |
| F | ♂G7-1 | Blank |
| G | ♂G7-2 | Blank |
| H | ♂G7-3 | Normal mouse serum (1:2000) |

The ELISA results show that the antibody to the corresponding outer membrane protein can be detected in the sera of animals infected with the homologous strain P. gingivalis. For example, mice infected with P. gingivalis subtype RagB-2, as shown in Table 5, consisted of a total of 8 mice (G6-1, G6-2, G7-1, G7-2, G8-1, G8-2, G8-3 and G8-4). Their sera reacted with the RagB-2 recombinant protein, indicating that the RagB-2 protein is a major antigen on the surface of bacteria. The results also showed that the higher the number of infections, the higher the corresponding antibody level, e.g., 4 mice in the G8 group (G8-1, G8-2, G8-3, G8-4) were challenged with P. gingivalis only once, while the other 4 mice (G6-1, G6-2, G7-1, G7-2) were infected with P. gingivalis 3 times. The OD value of antibody detection was higher in those infected multiple times than those infected only once. In addition, when animals with a history of P. gingivalis infection were immunized with RagB-2 protein, high titers of specific antibodies were produced (Table 4 and 5, G6-3, G6-4, G7-3, and G7-4). The OD values were substantially higher in animals following bacterial infection compared to those who had only received an active immunization vaccine regimen. This indicates that bacterial infection has caused animals to be in an immunosensitized state, and a vaccine product will promote effective immune response in patients with a history of prior infection.

TABLE 5
Experimental Animal's Information

| | Received RagB-2 | Number of attacks of |
|---|---|---|
| | Vaccine | P. gingivalis RagB-2 |
| ♂G6-1 | No | 3 |
| ♂G6-2 | No | 3 |
| ♂G6-3 | Yes | 3 |
| ♂G6-4 | Yes | 3 |
| ♂G7-1 | No | 3 |
| ♂G7-2 | No | 3 |
| ♂G7-3 | Yes | 3 |
| ♂G7-4 | Yes | 3 |
| ♂G8-1 | No | 1 |
| ♂G8-2 | No | 1 |
| ♂G8-3 | No | 1 |
| ♂G8-4 | No | 1 |

These results show the reaction in animals that were pre-infected with P. gingivalis RagB-2 upon immunization with RagB-2 protein (G6-3, G6-4, G7-3 and G7-4). The resulting sera showed not only a strong immune reaction to RagB-2, but also cross-reactivity to different subtypes of RagB including RagB-1, RagB-3 and RagB-4, and weak but visible cross-reactivity with the Cra4S1 protein. There was no reaction to the negative control GST protein.

TABLE 6
P. gingivalis Infected Animal's Polyclonal Antibody Detection

| 17004 | RagB-1 | RagB-2 | RagB-3 | RagB-4 | Cra4S1 | GST | RagB-1 | RagB-2 | RagB-3 | RagB-4 | Cra4S1 | GST |
|---|---|---|---|---|---|---|---|---|---|---|---|---|
| Serial | 1 | 2 | 3 | 4 | 5 | 6 | 7 | 8 | 9 | 10 | 11 | 12 |
| A | 0.04 | 0.045 | 0.038 | 0.038 | 0.043 | 0.036 | 0.11 | 0.101 | 1.883 | 0.248 | 0.415 | 0.053 |
| B | 0.048 | 0.049 | 0.279 | 0.042 | 0.054 | 0.038 | 0.044 | 0.049 | 0.17 | 0.046 | 0.052 | 0.042 |
| C | 0.045 | 0.052 | 0.411 | 0.043 | 0.054 | 0.039 | 0.047 | 0.046 | 0.217 | 0.044 | 0.093 | 0.043 |
| D | 0.075 | 0.351 | 2.287 | 0.552 | 0.201 | 0.049 | 0.052 | 0.058 | 0.242 | 0.053 | 0.068 | 0.048 |
| E | 0.104 | 0.163 | 1.177 | 0.32 | 0.631 | 0.051 | 0.043 | 0.048 | 0.078 | 0.048 | 0.06 | 0.038 |
| F | 0.048 | 0.073 | 0.441 | 0.147 | 0.061 | 0.038 | — | — | — | — | — | — |
| G | 0.046 | 0.054 | 0.471 | 0.062 | 0.063 | 0.038 | — | — | — | — | — | — |
| H | 0.073 | 0.197 | 1.694 | 0.441 | 0.279 | 0.039 | 0.039 | 0.041 | 0.039 | 0.048 | 0.048 | 0.036 |
| lay out | 1 | 2 | 3 | 4 | 5 | 6 | 7 | 8 | 9 | 10 | 11 | 12 |

| | | |
|---|---|---|
| A | PBS [symbol] | ♂G11-4 |
| B | ♂G9-1 | ♂G12-1 |
| C | ♂G9-2 | ♂G12-2 |
| D | ♂G9-3 | ♂G12-3 |
| E | ♂G9-4 | ♂G12-4 |
| F | ♂G11-1 | Blank |
| G | ♂G11-2 | Blank |
| H | ♂G11-3 | Normal mouse serum (1:2000) |

TABLE 7
Experimental Animal's Information

| | Received RagB-3 | Number of attacks of |
|---|---|---|
| | Vaccine | P. gingivalis RagB-3 |
| ♂G9-1 | No | 3 |
| ♂G9-2 | No | 3 |
| ♂G9-3 | Yes | 3 |
| ♂G9-4 | Yes | 3 |
| ♂G11-1 | No | 3 |
| ♂G11-2 | No | 3 |
| ♂G11-3 | Yes | 3 |
| ♂G11-4 | Yes | 3 |
| ♂G12-1 | No | 1 |
| ♂G12-2 | No | 1 |
| ♂G12-3 | No | 1 |
| ♂G12-4 | No | 1 |

Similarly, it was observed that immunization of pre-infected animals with RagB-3 protein also produced high-intensity specific antibodies, and the cross-reactivity of serum antibodies with RagB-1, RagB-2, RagB-4, and Cra4S1 (Table 6 and Table 7).

Therefore, this Example demonstrates that P. gingivalis recombinant outer membrane proteins including RagB and Cra4S1 have good antigenicity and can stimulate animals to produce high titers of antibodies. In addition, cross-reactivity of polyclonal serum antibodies with other subtypes of RagB proteins was observed. Although the gene sequence and protein sequence of P. gingivalis outer membrane protein RagB are significantly different between subtypes, they are homologous alleles. It is suggested that there are common antigens or compatible antigenic determinants between these subtypes of P. gingivalis. Particularly, the finding of antibodies produced by the RagB protein that are capable of cross-reacting with the Cra4S1 protein is surprising and unexpected.

### Example 3

### Cross-Reactivity of Monoclonal Antibodies to RagB

The monoclonal antibodies against P. gingivalis outer membrane proteins including RagB and Cra4S1 proteins were prepared respectively by the third-party CRO companies. Specifically, the general process of obtaining a monoclonal antibody by preparing mouse hybridoma cells is briefly described as follows: firstly, animals were immunized with antigenic proteins five times, and each immunization interval was 1-2 weeks. Murine splenocytes were fused with well-grown SP2/0 myeloma cells, and the cells were grown for seven days after fusion. The positive clones were then screened with immunization antigens, and further subcloned three times to confirm that the positive cell lines continued to stably produce antibodies. The amplified hybridoma seed cell lines were stored in liquid nitrogen.

TABLE 8
RagB hybridoma cell lines

| | | Recombinant protein cross-reaction | | | | |
|---|---|---|---|---|---|---|
| CRO | Hybridoma Name | RagB-1 | RagB-2 | RagB-3 | RagB-4 | Ig Subclass |
| HuaBio | 1A1-7-7 | P | — | — | — | IgG2a, κ |
| China | 1A3-3-8 | P | — | — | — | IgG1, κ |
| HAMJ0550 | 1A6-6-5 | P | — | — | — | IgG1, κ |
| RagB-1 | 1C3-3-3 | P | — | — | — | IgG1, κ |
| | 1E3-4-7-1 | P | — | — | — | IgG1, κ |
| | 1A1-5-5-5 | P | — | — | — | N/A |
| HuaBio | 1A3-4-1-7 | — | P | — | — | IgG1, κ |
| China | 1A4-3-7-7 | P | P | — | — | IgG1, κ |
| HAMJ0551 | 1A5-4-1-5 | — | P | — | — | IgG1, κ |
| RagB-2 | 1A6-3-3 | — | P | — | — | IgG1, κ |
| | 1A9-8-2-8 | — | P | — | — | IgG1, κ |
| | 1B3-1-5 | — | P | — | — | IgG2b, κ |
| HuaBio | 1C10-8-7-3 | — | — | P | — | IgG1, κ |
| China | 1D2-2-1-3 | P | P | P | — | IgG2a, κ |
| HAMJ0552 | 1D9-6-8-6 | — | — | P | — | IgG1, κ |
| RagB-3 | 1E4-6-1-7 | — | — | P | P | N/A |
| | 1E6-3-8-6 | — | — | P | — | N/A |
| | 1F10-5-3-6 | — | — | P | — | N/A |
| | 1H11-2-2-4 | — | — | P | P | IgG2a, κ |
| | 2B2-8-5-5 | — | — | P | — | N/A |
| | 2B7-4-6-7 | P | — | P | — | IgG1, κ |
| | 2B8-1-8-4 | — | — | P | — | N/A |
| | 2C1-4-6-2 | P | — | P | — | IgG1, κ |
| | 2C4-6-1-4 | — | — | P | — | N/A |
| | 2C5-4-6-6 | — | — | P | — | N/A |
| | 2C8-7-6-6 | — | — | P | — | IgG1, κ |
| | 2C10-1-2-3 | — | — | P | — | N/A |
| | 2E6-4-4-8 | — | — | P | — | N/A |
| HuaBio | 1B6-1-8 | — | — | — | P | IgG1, κ |
| China | 1B10-6-8 | — | — | — | P | IgG1, κ |
| HAMJ0242 | 1B11-4-4 | P | P | P | P | IgG1, κ |
| RagB-4 | 1C3-7-8 | P | P | P | P | IgG1, κ |
| | 1C11-1-5 | — | — | — | — | N/A |
| | 1D6-3-8 | — | — | — | P | IgG1, κ |
| | 1E2-3-7 | — | — | — | P | IgG1, κ |
| | 1D10-2-8 | — | — | — | P | IgG1, κ |

TABLE 9
Cra4S1 Hybridoma Cell Lines

| Hybridoma Name | | | | | | | | | | | | | |
|---|---|---|---|---|---|---|---|---|---|---|---|---|---|
| (Genecreate, | | | | | | | | | | | | | |
| China, PA1- | 4G4- | 5C2- | 7D5- | 5H1- | 5F9- | 6F10- | 1D12- | 2B10- | 3F1- | 4A3- | 4C2- | 8B1- | 5B9- |
| 21030025) | 1G11 | 2B10 | 3D9 | 2D11 | 4C8 | 4E11 | 4D2 | 5E7 | 1A8 | 2C9 | 2H5 | 5C5 | 2C5 |
| Ig Subclass | TgG2b | TgG1 | TgG1 | TgG2b | TgG1 | TgG1 | TgG2b | TgG1 | TgG1 | TgG1 | TgG1 | TgG1 | TgG1 |

Five to ten antibody-positive hybridoma cell lines for each antigen project were provided (Table 8 and Table 9). Monoclonal antibodies were mainly purified from cell culture supernatant and ascitic fluids produced after mouse hybridoma cells were implanted into the peritoneal cavity. Immunoglobulin subclass of the purified monoclonal antibodies were detected. Positive result labeled “P” in the Tables.

TABLE 10
Monoclonal Antibody Cross-Antigen Detection

| | RagB-1 | RagB-2 | RagB-3 | RagB-4 | RagB-1 | RagB-2 | RagB-3 | RagB-4 | RagB-1 | RagB-2 | RagB-3 | RagB-4 |
|---|---|---|---|---|---|---|---|---|---|---|---|---|
| 2101M-1 | 1 | 2 | 3 | 4 | 5 | 6 | 7 | 8 | 9 | 10 | 11 | 12 |
| A | 0.665 | 0.066 | 0.067 | 0.063 | 0.06 | 0.147 | 0.057 | 0.058 | 0.061 | 0.06 | 0.175 | 0.061 |
| B | 0.252 | 0.064 | 0.062 | 0.063 | 0.061 | 0.345 | 0.057 | 0.056 | 0.08 | 0.065 | 0.387 | 0.107 |
| C | 0.43 | 0.061 | 0.058 | 0.056 | 0.062 | 0.649 | 0.053 | 0.061 | 0.057 | 0.059 | 0.103 | 0.057 |
| D | 0.382 | 0.062 | 0.058 | 0.06 | 0.063 | 0.06 | 0.219 | 0.057 | 0.136 | 0.061 | 0.226 | 0.061 |
| E | 0.278 | 0.059 | 0.058 | 0.059 | 0.421 | 0.158 | 0.525 | 0.057 | 0.062 | 0.059 | 0.189 | 0.055 |
| F | 0.061 | 0.23 | 0.053 | 0.055 | 0.06 | 0.054 | 0.334 | 0.061 | 0.174 | 0.058 | 0.236 | 0.052 |
| G | 0.213 | 0.263 | 0.105 | 0.104 | 0.056 | 0.055 | 0.335 | 0.168 | 0.213 | 0.247 | 0.214 | 0.263 |
| H | 0.052 | 0.236 | 0.056 | 0.053 | 0.049 | 0.047 | 0.123 | 0.042 | 0.062 | 0.067 | 0.068 | 0.079 |
| Antibody | | | | | | | | | | | | |
| lay out | 1 | 2 | 3 | 4 | 5 | 6 | 7 | 8 | 9 | 10 | 11 | 12 |

| | | | |
|---|---|---|---|
| A | RagB-1-1A1-7-7 | RagB-2-1A6-3-3 | RagB-3-1F10-5-3-6 |
| B | RagB-1-1A3-3-8 | RagB-2-1A9-8-2-8 | RagB-3-1H11-2-2-4 |
| C | RagB-1-1A6-6-5 | RagB-2-1B3-1-5 | RagB-3-2B2-8-5-5 |
| D | RagB-1-1C3-3-3 | RagB-3-1C10-8-7-3 | RagB-3-2B7-4-6-7 |
| E | RagB-1-1E3-4-7-1 | RagB-3-1D2-2-1-3 | RagB-3-2B8-1-8-4 |
| F | RagB-2-1A3-4-1-7 | RagB-3-1D9-6-8-6 | RagB-3-2C1-4-6-2 |
| G | RagB-2-1A4-3-7-7 | RagB-3-1E4-6-1-7 | Positive control |
| H | RagB-2-1A5-4-1-5 | RagB-3-1E6-3-8-6 | Normal mouse serum control |

TABLE 11
Monoclonal Antibody Cross-Antigen Detection

| | RA | RB | RC | RD | RA | RE | RC | RD | RA | RB | RC | RD |
|---|---|---|---|---|---|---|---|---|---|---|---|---|
| 2101M-2 | 1 | 2 | 3 | 4 | 5 | 6 | 7 | 8 | 9 | 10 | 11 | 12 |
| A | 0.062 | 0.064 | 0.251 | 0.06 | 0.296 | 0.122 | 0.352 | 0.414 | 0.364 | 0.063 | 0.063 | 0.062 |
| B | 0.084 | 0.058 | 0.255 | 0.066 | 0.06 | 0.059 | 0.065 | 0.064 | 0.061 | 0.167 | 0.064 | 0.062 |
| C | 0.063 | 0.058 | 0.373 | 0.063 | 0.061 | 0.057 | 0.058 | 0.341 | 0.471 | 0.133 | 0.554 | 0.094 |
| D | 0.065 | 0.059 | 0.282 | 0.063 | 0.087 | 0.079 | 0.103 | 0.143 | 0.403 | 0.118 | 0.401 | 0.099 |
| E | 0.061 | 0.058 | 0.223 | 0.062 | 0.534 | 0.196 | 0.726 | 0.124 | 0.131 | 0.166 | 0.075 | 0.078 |
| F | 0.058 | 0.053 | 0.057 | 0.246 | 0.344 | 0.151 | 0.662 | 0.095 | 0.117 | 0.142 | 0.067 | 0.067 |
| G | 0.056 | 0.053 | 0.057 | 0.402 | 0.334 | 0.107 | 0.522 | 0.087 | 0.225 | 0.285 | 0.224 | 0.261 |
| H | 0.284 | 0.225 | 0.254 | 0.48 | 0.252 | 0.065 | 0.345 | 0.056 | 0.074 | 0.066 | 0.072 | 0.075 |
| Antibody | | | | | | | | | | | | |
| lay out | 1 | 2 | 3 | 4 | 5 | 6 | 7 | 8 | 9 | 10 | 11 | 12 |

| | | | |
|---|---|---|---|
| A | RagB-3-2C4-6-1-4 | 1C3-7-8 | RagB-1-1C3-3-3 |
| B | RagB-3-2C5-4-6-6 | 1C11-1-5 | RagB-2-1A6-3-3 |
| C | RagB-3-2C8-7-6-6 | 1D6-3-8 | 1D2-2-1-3 purified ascites 1:2000 |
| D | RagB-3-2C10-1-2-3 | 1E2-3-7 | 1D2-2-1-3 purified ascites 1:4000 |
| E | RagB-3-2E6-4-4-8 | GM1D2-2-1-3 1:2000 | 1A4-3-7-7 purified ascites 1:2000 |
| F | RagB-4-1D10-2-8 | GM1D2-2-1-3 1:4000 | 1A4-3-7-7 purified ascites 1:4000 |
| G | RagB-4-1B10-6-8 | GM1D2-2-1-3 1:8000 | Positive control |
| H | RagB-4-1B11-4-4 | GM1D2-2-1-3 1:16000 | Normal mouse serum control |

Table 10 and 11 show the original results of cross-reactivity of monoclonal antibodies and RagB recombinant proteins. The ELISA procedure is as described in Example 2. The antigen-coated plates were designed as follows: 1st, 5th, and 9th columns were coated with RagB-1 recombinant protein; 2nd, 6th, and 10th columns were coated with RagB-2; 3rd, 7th, and 11th columns were coated with RagB-3; and 4th, 8th and 12th columns were coated with RagB-4. The antibodies were in the hybridoma culture supernatants or purified antibodies were diluted 1:1000~1:4000. Each antibody sample was able to react with 4-well antigen proteins RagB-1, RagB-2, RagB-3 and RagB-4.

Compared with the normal serum negative control group, if the OD reading value was more than double that of the control group, it is considered as positive. The data presented in this Example illustrates that all tested monoclonal antibodies, except for one hybridoma (well B5-8, cell name RagB-4-1C11-1-5 listed in Table 11 showed negative reaction), showed positive immunization against the antigen. Moreover, several hybridoma antibodies were identified that not only reacted with their corresponding RagB protein, but also cross-reacted with other subtypes of RagB proteins.

For example, in Table 10, plate positions G1-G4, RagB-2-1A4-3-7-7 is a specific monoclonal antibody against RagB-2 protein, but also cross-reacted with RagB-1 protein; plate position E5-E8, RagB-3-1D2-2-1-3 is a specific monoclonal antibody against RagB-3 protein, but also cross-reacted with RagB-1 and RagB-2 proteins. In Table 11, positions H1-H4 and A5-A8, 1B11-4-4 antibody and 1C3-7-8 antibodies are RagB-4 protein-specific monoclonal antibodies, however, they both cross-reacted with RagB-1, RagB-2, and RagB-3 proteins, respectively.

Since the nucleic acid and protein sequences between the RagB subtypes of P. gingivalis are significantly different with no large contiguous common sequences in pairwise alignments, the conventional bacterial immunotherapy was to focus on precision, starting with identifying a subtype first, then selecting a specific monoclonal antibody against the corresponding subtype. However, this Example provides some monoclonal antibodies that are capable of cross-reacting with different subtypes of RagB proteins, suggesting that the different subtypes of P. gingivalis share common targets or compatible epitopes. Then, what and where are these common targets and/or compatible epitopes?

### Example 4

### Targets of Monoclonal Antibodies in Peptide Libraries

To identify the antigenicity of P. gingivalis Rag outer membrane proteins and the antigenic targets bound by monoclonal antibodies, five peptide libraries for outer membrane proteins RagB-1, RagB-2, RagB-3, RagB-4, and Cra4S1 were established. The synthetic peptides were produced by the third-party CRO Company (GenScript, USA). Specifically, based on the amino acid sequence of each protein, starting from the N-terminus, an 18-25 amino acid peptide chain was artificially synthesized. The next peptide would contain an overlap of five amino acids from the C-terminus of the previous peptide chain, and so on. Aside from the peptides at both ends of each protein, which each have a single overlapping sequence, all other peptides have a five amino acid overlap with the upstream and downstream peptide chains to either side, to avoid the omission of any antigenic determinants. The peptide sequences are shown in Tables 12-16. Each subtype of RagB protein is segmented into 24 peptide chains, and the peptides of RagB-1 are named RA plus a serial number denoting its location, for example, RA-1, RA-2 . . . , and so on. The peptides of RagB-2 are named RB plus the sequence number, the peptides of RagB-3 are named RC plus the sequence number, and the peptides of RagB-4 are named RD plus the sequence number. The peptides of Cra4S1 are named Cra and New Cra plus the sequence number. Most of the peptide fragments are soluble in ultrapure water, PBS and/or DMSO. The peptide sequences and the physical and chemical properties of the peptide chains are listed in Tables 12-16.

After the P. gingivalis outer membrane protein peptide libraries were established, all monoclonal antibodies were reacted with the fragments in the peptide libraries to find the target sites and key sequence segments for antigen-antibody binding. Specifically, a soluble coating solution (1 mg/ml) was prepared according to the solubility of the peptide, and then the concentration of the peptide solution was further diluted to 20 μg/ml, and the ELISA plate was coated. The subsequent antibody detection steps were as described in Example 2. Each monoclonal antibody was reacted with its corresponding RagB subtype outer membrane protein peptide fragments. To avoid misdiagnosing peptide antigens and properly determine fragment alignment, each synthetic peptide has overlapping portions with upstream and downstream peptides as previously described.

Four established RagB subtype libraries are designed to capture epitopes and cross-antigen targets with monoclonal antibodies. Some monoclonal antibodies to one RagB subtype showed cross-reactivity to another RagB subtype as described in Example 3. These antibodies were then also screened against the peptide library of the RagB subtype where cross reactivity was identified, to try to locate the antibody-antigen target site.

TABLE 12
RagB-1 (SEQ ID NO: 2) peptide library (GenScript, USA, C4965GB230)

| | | | | Solvent | | | |
|---|---|---|---|---|---|---|---|
| | | | | selection | | | |
| Peptide | Peptide | AA | | ultrapure | 0.1M | | |
| Name | sequence | Number | Purity | H₂O | PBS | DMSO | Notes |
| RA-1 | ELDRDPEGKD FQQPYTSFVQ | 25 | 85.0% | Y | Y | Y | |
| | TKQNR (SEQ ID NO: 11) | | | | | | |
| RA-2 | TKQNRDGLYA LLRNTENPRM | 25 | 96.0% | Y | Y | Y | |
| | HFYQE (SEQ ID NO: 12) | | | | | | |
| RA-3 | HFYQELQSDM YCTTITDGN | 11 | 96.7% | — | Y | Y | Low isoelectric |
| | S LAPFV (SEQ ID NO: 13) | | | | | | point, difficult |
| | | | | | | | to purify, |
| | | | | | | | only the |
| | | | | | | | bold sequence |
| RA-4 | LAPFVNWDLG ILNDHGRADE | 25 | 85.9% | Y | Y | Y | |
| | DEVSG (SEQ ID NO: 14) | | | | | | |
| RA-5 | DEVSGIAGYY FVYNRLNQQA | 25 | 87.8% | | — | Y | |
| | NAFVN (SEQ ID NO: 15) | | | | | | |
| RA-6 | NAFVNNTEAA LQNQVYKNST | 25 | 70.1% | — | - | Y | |
| | EIANA (SEQ ID NO: 16) | | | | | | |
| RA-7 | EIANAKSFLA EGKVLQALAI | 25 | 91.6% | — | - | Y | |
| | WRLMD (SEQ ID NO: 17) | | | | | | |
| RA-8 | WRLMDRFSFH ESVTEVNSGA | 25 | 82.3% | — | - | Y | |
| | KDLGV (SEQ ID NO: 18) | | | | | | |
| RA-9 | KDLGVILLKE YNPGYIGPRA | 25 | 91.9% | Y | Y | Y | |
| | TKAQC (SEQ ID NO: 19) | | | | | | |
| RA-10 | TKAQCYDYIL SRLSEAIEVL | 25 | 86.5% | — | - | Y | |
| | PENRE (SEQ ID NO: 20) | | | | | | |
| RA-11 | PENRESVLYV SRDYAYALRA | 25 | 76.4% | Y | — | Y | |
| | RIYLA (SEQ ID NO: 21) | | | | | | |
| RA-12 | RIYLALGEYG KAAADAKMVV | 25 | 85.3% | Y | — | - | |
| | DKYPL (SEQ ID NO: 22) | | | | | | |
| RA-13 | DKYPLIGAAD ASEFENIYRS | 25 | 90.2% | | — | Y | |
| | DANNP (SEQ ID NO: 23) | | | | | | |
| RA-14 | DANNPEIIFR GFASATLGSF | 10 | 94.9% | Y | — | Y | The sequence is |
| | TATTL (SEQ ID NO: 24) | | | | | | hydrophobic and |
| | | | | | | | the amino acid |
| | | | | | | | coupling |
| | | | | | | | efficiency is |
| | | | | | | | low, only the |
| | | | | | | | bold sequence |
| RA-15 | TATTLNGAAP AGKDIKYNPS | 25 | 94.7% | Y | Y | Y | |
| | AVPFQ (SEQ ID NO: 25) | | | | | | |
| RA-16 | AVPFQWVVDL YENEDFRKSV | 25 | 75.8% | Y | Y | Y | |
| | YIAKV (SEQ ID NO: 26) | | | | | | |
| RA-17 | YIAKVVKKDK GYLVNKFLED | 25 | 81.9% | Y | Y | Y | |
| | KAYRD (SEQ ID NO: 27) | | | | | | |
| RA-18 | KAYRDVQDKP NLKVGARYFS | 25 | 73.8% | Y | Y | Y | |
| | VAEVY (SEQ ID NO: 28) | | | | | | |
| RA-19 | VAEVYLILVE SALQTGDTPT | 25 | 70.2% | — | | Y | |
| | AEKYL (SEQ ID NO: 29) | | | | | | |
| RA-20 | AEKYLKALSK ARGAEVSWVN | 25 | 81.6% | Y | — | Y | |
| | MEALQ (SEQ ID NO: 30) | | | | | | |
| RA-21 | MEALQAERTR ELIGEGSRLR | 25 | 91.3% | Y | — | Y | |
| | DMVRW (SEQ ID NO: 31) | | | | | | |
| RA-22 | DMVRWSIPNN HDAFETQPGL | 25 | 82.5% | Y | — | Y | The amino acid |
| | EGFAN (SEQ ID NO: 32) | | | | | | coupling |
| | | | | | | | efficiency |
| | | | | | | | is low |
| RA-23 | EGFANTTPLK AQAPVGFYAY | 25 | 89.5% | Y | Y | Y | |
| | TWEFP (SEQ ID NO: 33) | | | | | | |
| RA-24 | TWEFPQRDRQ TNPQLIKNWP | 21 | 99.6% | Y | Y | Y | |
| | I (SEQ ID NO: 34) | | | | | | |

TABLE 13
RagB-2 (SEQ ID NO: 4) peptide library (GenScript, USA, C4965GB230)

| | | ino | | Solvent selection | | | |
|---|---|---|---|---|---|---|---|
| Peptide | | Acid | | ultrapure | 0.1 M | | |
| Name | Peptide sequence | Num | Purity | H₂O | PBS | DMSO | Notes |
| RB-1 | CDLDRTPHNS DVQKPYEDMA | 25 | 75.3% | Y | Y | Y | |
| | TTVQY (SEQ ID NO: 35) | | | | | | |
| RB-2 | TTVQYRDGLY SVLRGAENAG | 25 | 70.4% | — | — | Y | |
| | RYTLS (SEQ ID NO: 36) | | | | | | |
| RB-3 | RYTLSEYMSD MYCVMQGDGG | 25 | 95.1% | — | — | Y | |
| | HATPY (SEQ ID NO: 37) | | | | | | |
| RB-4 | HATPYVTWTI PRIEIADHAS | 25 | 87.5% | — | — | Y | |
| | NYYFG (SEQ ID NO: 38) | | | | | | |
| RB-5 | NYYFGFNRLI QQANAFVGNV | 25 | 70.3% | — | — | Y | |
| | KLAIA (SEQ ID NO: 39) | | | | | | |
| RB-6 | KLAIANGVYK TEVDKTNAQI | 25 | 90.2% | Y | — | Y | |
| | YLAEA (SEQ ID NO: 40) | | | | | | |
| RB-7 | YLAEAKTLQA LALFRLMERF | 25 | 85.6% | — | — | Y | |
| | AYPYD (SEQ ID NO: 41) | | | | | | |
| RB-8 | AYPYDPNETT SPKNLGVVLI | 25 | 91.8% | Y | Y | Y | |
| | KEYDP (SEQ ID NO: 42) | | | | | | |
| RB-9 | KEYDPWAVGA RATQTETYSY | 25 | 81.6% | — | — | Y | |
| | IMSLL (SEQ ID NO: 43) | | | | | | |
| RB-10 | IMSLLDEAIS VLPETNANNM | 25 | 91.1% | — | — | Y | |
| | YVSRD (SEQ ID NO: 44) | | | | | | |
| RB-11 | YVSRDYALGL RARVHMAMDN | 25 | 74.3% | — | — | Y | |
| | YAEAA (SEQ ID NO: 45) | | | | | | |
| RB-12 | YAEAANDIRA FYKKYNLISA | 25 | 88.0% | Y | Y | Y | |
| | ANSDE (SEQ ID NO: 46) | | | | | | |
| RB-13 | ANSDEFEEAY RKMSSNPELI | 25 | 91.8% | Y | Y | Y | |
| | FRGYA (SEQ ID NO: 47) | | | | | | |
| RB-14 | FRGYASVTNG YLVYQDLMGA | 25 | 85.5% | — | — | Y | |
| | TASGT (SEQ ID NO: 48) | | | | | | |
| RB-15 | TASGTNVKYN PRVTPLQWVC | 25 | 98.4% | Y | Y | Y | |
| | DLYDA (SEQ ID NO: 49) | | | | | | |
| RB-16 | DLYDAADYRK KVYIVDKVNG | 25 | 99.3% | Y | — | Y | |
| | DGGKG (SEQ ID NO: 50) | | | | | | |
| RB-17 | DGGKGYWNK FLGDPELRED | 25 | 98.5% | Y | Y | Y | |
| | PKKEN (SEQ ID NO: 51) | | | | | | |
| RB-18 | PKKENFKTGC RFFSLAEAYL | 25 | 76.7% | — | — | — | HCOOH |
| | ILAEA (SEQ ID NO: 52) | | | | | | |
| RB-19 | ILAEADIMTG NTAEAMEVLK | 25 | 90.4% | — | — | — | 3% ammonia |
| | ELSKS (SEQ ID NO: 53) | | | | | | water |
| RB-20 | ELSKSRGAEV SGADYMQILK | 25 | 76.3% | — | — | Y | |
| | DERTR (SEQ ID NO: 54) | | | | | | |
| RB-21 | DERTREMIGE GSRLNDMIRW | 25 | 97.5% | Y | — | Y | |
| | NMDLV (SEQ ID NO: 55) | | | | | | |
| RB22 | NMDLVVSPVQ AVLHKIAVPT | 25 | 82.1% | Y | Y | Y | |
| | ILQTD (SEQ ID NO: 56) | | | | | | |
| RB-23 | ILQTDDPTRV PAGFYAFTWE | 25 | 93.5% | | | Y | |
| | IPNRD (SEQ ID NO: 57) | | | | | | |
| RB-24 | IPNRDLVVIP ELVRNWPKQ | 19 | 96.2% | Y | Y | Y | |
| | (SEQ ID NO: 58) | | | | | | |

TABLE 14
RagB-3 (SEQ ID NO: 6) peptide library (GenScript, USA, C5903GB210)

| | | Amino | | | | | |
|---|---|---|---|---|---|---|---|
| Peptide | | Acid | | Solvent selection | | | Notes |
| Name | Peptide sequence | Number | Purity | ultrapure H₂O | 0.1 M PBS | DMSO | |
| RC-1 | LNRDPEDKAK KEPFKTIAQA | 25 | 79.5% | Y | Y | Y | |
| | RQSRD (SEQ ID NO: 59) | | | | | | |
| RC-2 | RQSRDGLYSL LRESEQLAFH | 25 | 80.1% | — | — | Y | |
| | VYDEV (SEQ ID NO: 60) | | | | | | |
| RC-3 | VYDEVQSDMY TTTKNDGNQY | 25 | 84.4% | Y | — | Y | |
| | YPFVA (SEQ ID NO: 61) | | | | | | |
| RC-4 | YPFVAWQLGS METHGRADED | 25 | 91.4% | — | — | Y | |
| | QVYGI (SEQ ID NO: 62) | | | | | | |
| RC-5 | QVYGIAGYYF EYSRLIQQAN | 25 | 90.5% | — | — | — | 3% ammonia water |
| | SYIED (SEQ ID NO: 63) | | | | | | |
| RC-6 | SYIEDMEKAL ANNTDMSIFI | 19 | 70.3% | — | — | — | Low isoelectric |
| | SSEEV (SEQ ID NO: 64) | | | | | | point, |
| | | | | | | | difficult to |
| | | | | | | | purify, only |
| | | | | | | | the bold |
| | | | | | | | sequence |
| RC-7 | SSEEVERAKR YLAEVRVVQA | 25 | 95.1% | Y | — | Y | |
| | LANWR (SEQ ID NO: 65) | | | | | | |
| RC-8 | LANWRLMDRF AYKYDAASNQ | 25 | 94.5% | Y | — | Y | |
| | SPQNL (SEQ ID NO: 66) | | | | | | |
| RC-9 | SPQNLGIVLI KEFNPAYIGP | 25 | 85.9% | — | — | Y | |
| | RATQK (SEQ ID NO: 67) | | | | | | |
| RC-10 | RATQKECYDH ILSALEAAIT | 25 | 73.5% | Y | Y | Y | |
| | VLPEK (SEQ ID NO: 68) | | | | | | |
| RC-11 | VLPEKNPDGI TYAGRDYAHA | 25 | 91.5% | Y | Y | Y | |
| | LRARV (SEQ ID NO: 69) | | | | | | |
| RC-12 | LRARVHLSMG NYADALADAK | 25 | 79.4% | Y | — | Y | |
| | QIVEK (SEQ ID NO: 70) | | | | | | |
| RC-13 | QIVEKYPLIN ANNAEDFAKI | 25 | 90.2% | Y | Y | Y | |
| | YRSDA (SEQ ID NO: 71) | | | | | | |
| RC-14 | YRSDANNPEI VFRAFASPTT | 25 | 96.5% | Y | Y | Y | |
| | GAVGA (SEQ ID NO: 72) | | | | | | |
| RC-15 | GAVGATSLNG ASVVNKKIKY | 25 | 74.7% | Y | Y | Y | |
| | APFIV (SEQ ID NO: 73) | | | | | | |
| RC-16 | APFIVPLQWV VDLYEDADYR | 25 | 84.1% | | — | Y | |
| | KSVYI (SEQ ID NO: 74) | | | | | | |
| RC-17 | KSVYIDKTVS NGSEKGYLVN | 25 | 89.7% | Y | — | Y | |
| | KFLED (SEQ ID NO: 75) | | | | | | |
| RC-18 | KFLEDPAYRE TADIPILKIG | 25 | 74.2% | — | — | Y | |
| | VRMFS (SEQ ID NO: 76) | | | | | | |
| RC-19 | VRMFSIAEAY LMVAECAHMT | 25 | 90.5% | — | — | — | HCOOH |
| | GDDAT (SEQ ID NO: 77) | | | | | | |
| RC-20 | GDDATAIAYL KKLSAARGGN | 25 | 85.0% | Y | Y | Y | |
| | IDTGD (SEQ ID NO: 78) | | | | | | |
| RC-21 | IDTGDVMKAI QEERTRELIG | 25 | 76.7% | Y | Y | Y | |
| | EGARL (SEQ ID NO: 79) | | | | | | |
| RC-22 | EGARLRDMIR WNLPNIDKTE | 25 | 84.4% | Y | Y | Y | |
| | IQPAL (SEQ ID NO: 80) | | | | | | |
| RC-23 | IQPALTGYAR EIVLEQPVPA | 25 | 91.5% | Y | Y | Y | |
| | GHYAF (SEQ ID NO: 81) | | | | | | |
| RC-24 | GHYAFTWEFP NRDRQVNPHL | 24 | 99.1% | Y | — | Y | |
| | IKNW (SEQ ID NO: 82) | | | | | | |

TABLE 15
RagB-4 (SEQ ID NO: 8) peptide library (GenScript, USA, C5903GB210)

| | | Amino | | Solvent selection | | | |
|---|---|---|---|---|---|---|---|
| Peptide | | Acid | | ultrapure | 0.1 M | | |
| Name | Peptide sequence | Number | Purity | H₂O | PBS | DMSO | Notes |
| RD-1 | MDPDGSDEQK DHFASFVETK | 25 | 92.8% | Y | Y | Y | |
| | HFRDG (SEQ ID NO: 83) | | | | | | |
| RD-2 | HFRDGLYATL RSTENPTRFV | 25 | 93.9% | — | — | Y | |
| | WQDLQ (SEQ ID NO: 84) | | | | | | |
| RD-3 | WQDLQSDMYA VTTNDGNTSS | 25 | 70.2% | — | — | Y | |
| | RFITW (SEQ ID NO: 85) | | | | | | |
| RD-4 | RFITWSLGAL ESSGEIASYY | 15 | 84.8% | — | — | Y | The sequence is |
| | LAYYS (SEQ ID NO: 86) | | | | | | hydrophobic, and |
| | | | | | | | the amino acid |
| | | | | | | | coupling |
| | | | | | | | efficiency is |
| RD-5 | LAYYSLLQRA NYFVTRIERS | 25 | 73.8% | Y | — | Y | |
| | MELNL (SEQ ID NO: 87) | | | | | | |
| RD-6 | MELNLYLEKE LKDVKIFQAE | 25 | 82.4% | — | — | Y | |
| | GKTLQ (SEQ ID NO: 88) | | | | | | |
| RD-7 | GKTLQALALS RLMERFAYKY | 25 | 88.6% | Y | — | Y | |
| | DPAAT (SEQ ID NO: 89) | | | | | | |
| RD-8 | DPAATTHPYD LGIVLVKDYN | 25 | 92.5% | — | — | Y | |
| | PMIAA (SEQ ID NO: 90) | | | | | | |
| RD-9 | PMIAAPRNTQ KECYDYILEC | 25 | 81.8% | Y | Y | Y | |
| | LNQAI (SEQ ID NO: 91) | | | | | | |
| RD-10 | LNQAIDVLPN KSNEGNIRVS | 25 | 88.4% | Y | Y | Y | |
| | KHYAH (SEQ ID NO: 92) | | | | | | |
| RD-11 | KHYAHALRAR VNFAMGNYDA | 25 | 79.3% | Y | — | Y | |
| | AKEDA (SEQ ID NO: 93) | | | | | | |
| RD-12 | AKEDAKVLVD NYPLIDVTTA | 25 | 86.9% | Y | Y | Y | |
| | KKFAE (SEQ ID NO: 94) | | | | | | |
| RD-13 | KKFAEVYRDD ANNPEIVFRA | 25 | 98.2% | Y | Y | Y | |
| | FASGT (SEQ ID NO: 95) | | | | | | |
| RD-14 | FASGTIGTVA ETTLSGFLWH | 25 | 87.1% | — | — | Y | |
| | SGAQL (SEQ ID NO: 96) | | | | | | |
| RD-15 | SGAQLVVSSP ISAPFQWWVD | 11 | 93.2% | — | — | Y | The sequence is |
| | LYDDT (SEQ ID NO: 97) | | | | | | hydrophobic, and |
| | | | | | | | the amino acid |
| | | | | | | | coupling |
| | | | | | | | efficiency is |
| | | | | | | | low; the |
| | | | | | | | isoelectric |
| | | | | | | | point is low, |
| | | | | | | | and |
| | | | | | | | the purification |
| | | | | | | | is difficult, |
| | | | | | | | only the |
| RD-16 | LYDDTDYRKS CYITKDFYVI | 25 | 78.4% | — | — | Y | |
| | GGGVD (SEQ ID NO: 98) | | | | | | |
| RD-17 | GGGVDKGYVV GKYLGNPAYQ | 25 | 89.6% | Y | Y | Y | |
| | SNPNV (SEQ ID NO: 99) | | | | | | |
| RD-18 | SNPNVPDFKV TSRFFSVAEA | 25 | 83.0% | — | — | Y | |
| | YLIMA (SEQ ID NO: 100) | | | | | | |
| RD-19 | YLIMAESMAK SGDAAGAKDL | 25 | 79.9% | — | — | Y | |
| | LKTLC (SEQ ID NO: 101) | | | | | | |
| RD-20 | LKTLCEKRGG QLEDGDIMDL | 25 | 88.8% | Y | Y | Y | |
| | VMAER (SEQ ID NO: 102) | | | | | | |
| RD-21 | VMAERTRELI GEGSRLNDMI | 25 | 72.3% | — | — | Y | |
| | RWNLP (SEQ ID NO: 103) | | | | | | |
| RD-22 | RWNLPNNHDD MENQPVFLQI | 25 | 95.4% | Y | — | Y | |
| | GLAKA (SEQ ID NO: 104) | | | | | | |
| RD-23 | GLAKADKLKQ PVPAGHYAFT | 25 | 99.5% | Y | — | Y | |
| | WEFPV (SEQ ID NO: 105) | | | | | | |
| RD-24 | WEFPVRDRQV NPQIIKNWPN | 20 | 94.9% | Y | Y | Y | |
| | (SEQ ID NO: 106) | | | | | | |

TABLE 16
Cra4S1 (SEQ ID NO: 10) peptide library

| | | | Amino | | Solvent selection | | | |
|---|---|---|---|---|---|---|---|---|
| | Peptide | | Acid | | ultrapure | 0.1 M | | |
| CRO | Name | Peptide sequence | Number | Purity | H₂O | PBS | DMSO | Notes |
| GenScript, | Cra-1 | QNRTVKGTVISSEDNEPL | 13 | 96.7% | Y | Y | Y | |
| USA | | (SEQ ID NO: 107) | | | | | | |
| C4965GB230 | Cra-2 | DNEPLIGANVVVVGNTTI | 18 | 96.5% | Y | Y | Y | |
| | | (SEQ ID NO: 108) | | | | | | |
| | Cra-3 | GNTTIGAATDLDGNFTLS | 18 | 95.6% | — | — | Y | |
| | | (SEQ ID NO: 109) | | | | | | |
| | Cra-4 | NFTLSVPANAKMLRVSYS | 18 | 95.9% | — | — | Y | |
| | | (SEQ ID NO: 110) | | | | | | |
| | Cra-5 | RVSYSGMTTKEVAIANVM | 18 | 98.3% | Y | x | Y | |
| | | (SEQ ID NO: 111) | | | | | | |
| | Cra-6 | IANVMKIVLDPDSKVLEQ | 18 | 95.3% | — | — | Y | |
| | | (SEQ ID NO: 112) | | | | | | |
| | Cra-7 | KVLEQVVVLGYGTGQKLS | 18 | 96.0% | Y | Y | Y | |
| | | (SEQ ID NO: 113) | | | | | | |
| | Cra-8 | GQKLSTVSGSVAKVSSEK | 18 | 95.1% | Y | Y | Y | |
| | | (SEQ ID NO: 114) | | | | | | |
| | Cra-9 | VSSEKLAEKPVANIMDAL | 18 | 96.3% | Y | Y | Y | |
| | | (SEQ ID NO: 115) | | | | | | |
| | Cra-10 | IMDALQGQVAGMQV | 14 | 95.7% | Y | Y | Y | |
| | | (SEQ ID NO: 116) | | | | | | |
| GenScript, | New Cra-1 | KGTVI SSEDNEPLIG | 25 | 71.3% | — | — | — | 3% ammonia |
| USA | | ANVVVVGNTT | | | | | | water |
| C935NGF150 | | (SEQ ID NO: 117) | | | | | | |
| | New Cra-2 | VGNTT IGAATDLDGN | 25 | 97.8% | Y | Y | Y | |
| | | FTLSVPANAK | | | | | | |
| | | (SEQ ID NO: 118) | | | | | | |
| | New Cra-3 | PANAK MLRVSYSGMT | 25 | 81.2% | Y | — | Y | |
| | | TKEVAIANVM | | | | | | |
| | | (SEQ ID NO: 119) | | | | | | |
| | New Cra-4 | IANVM KIVLDPDSKV | 25 | 92.2% | — | — | Y | |
| | | LEQVVVLGYG | | | | | | |
| | | (SEQ ID NO: 120) | | | | | | |
| | New Cra-5 | VLGYG TGQKLSTVSG | 25 | 91.4% | Y | Y | Y | |
| | | SVAKVSSEKL | | | | | | |
| | | (SEQ ID NO: 121) | | | | | | |
| | New Cra-6 | SSEKL AEKPVANIMD | 25 | 92.9% | Y | Y | Y | |
| | | ALQGQVAGMQ | | | | | | |
| | | (SEQ ID NO: 122) | | | | | | |

The antigen-antibody binding reactions between the monoclonal antibodies and the corresponding peptide fragments were determined according to the OD value of the indirect ELISA experiment. An OD value of double the OD of the negative control was regarded as a positive result.

Table 17 shows the peptide library for RagB-1(SEQ ID NO: 2), and the monoclonal antibody capture of antigenic targets, both by monoclonal antibodies directed at RagB-1 and monoclonal antibodies which were identified as reacting to other RagB subtypes. The ELISA results showed that peptide antigen targets were detected by 3 out of 5 hybridoma antibodies of RagB-1, e.g., monoclonal antibody 1A1-7-7 and 1A3-3-8 react with RA-1 peptide, 1E3-4-7-1 reacts with RA-9 peptide (Table 17). Mouse serum containing polyclonal antibodies raised from vaccination with RagB-1 protein was named 2019-G85-1 serum (P), which reacts with RA-1 peptide. Interestingly, other subtypes of monoclonal antibodies that cross-react with RagB-1 protein also found target antigens in the RagB-1 peptide library. For example, 1A4-3-7-7 (specific to RagB-2 protein) binds to RA-19 peptide; 1D2-2-1-3 (specific to RagB-3 protein) binds to RA-17 peptide, 2C1-4-6-2 (specific to RagB-3 protein) binds to RA-9 and RA-17, 2B7-4-6-7 (specific to RagB-3 protein) binds to RA9; and 1B11-4-4 (specific to RagB-4 protein) binds to RA-21 peptide.

TABLE 17
RagB-1 (SEQ ID NO: 2) Peptide Library Screening Targeted Antigen

| | | Antibodies to RagB-1 | | | | | | Cross-Reaction antobodies raised from other subtype of RagB | | | | | |
|---|---|---|---|---|---|---|---|---|---|---|---|---|---|
| | | | | | | 1C3-3-3 | 1E3-4-7-1 | | 1D2-2-1-3 | 2C1-4-6-2 | 2B7-4-6-7 | 1B11-4-4 | 1C3-7-8 |
| Peptide's Name and | | 2019-G85-1 | 1A1-7-7 | 1A3-3-8 | 1A6-6-5 | Super- | Super- | 1A4-3-7-7 | Recom- | Super- | Super- | Recom- | Recom- |
| amino acid Sequence | | Serum (P) | Ascites | Ascites | Ascites | natan | natant | Ascites | binant | natant | natant | binant | binant |
| RA-1 | ELDRDPEGKD FQQP | Positive | Positive | — | — | Positive | — | — | — | — | — | — | — |
| | YTSFVQ TKQNR | | | | | | | | | | | | |
| | (SEQ ID NO: 11) | | | | | | | | | | | | |
| RA-2 | TKQNRDGLYA LLRN | — | — | — | — | — | — | — | — | — | — | — | — |
| | TENPRM HFYQE | | | | | | | | | | | | |
| | (SEQ ID NO: 12) | | | | | | | | | | | | |
| RA-3 | HFYQELQSDM YCTT | — | — | — | — | — | — | — | — | — | — | — | — |
| | ITDGNS LAPFV | | | | | | | | | | | | |
| | (SEQ ID NO: 13) | | | | | | | | | | | | |
| RA-4 | LAPFVNWDLG ILND | — | — | — | — | — | — | — | — | — | — | — | — |
| | HGRADE DEVSG | | | | | | | | | | | | |
| | (SEQ ID NO: 14) | | | | | | | | | | | | |
| RA-5 | DEVSGIAGYY FVYN | — | — | — | — | — | — | — | — | — | — | — | — |
| | RLNQQA NAFVN | | | | | | | | | | | | |
| | (SEQ ID NO: 15) | | | | | | | | | | | | |
| RA-6 | NAFVNNTEAA LQNQ | — | — | — | — | — | — | — | — | — | — | — | — |
| | VYKNST EIANA | | | | | | | | | | | | |
| | (SEQ ID NO: 16) | | | | | | | | | | | | |
| RA-7 | EIANAKSFLA EGKV | — | — | — | — | — | — | — | — | — | — | — | — |
| | LQALAI WRLMD | | | | | | | | | | | | |
| | (SEQ ID NO: 17) | | | | | | | | | | | | |
| RA-8 | WRLMDRFSFH ESVT | — | — | — | — | — | — | — | — | — | — | — | — |
| | EVNSGA KDLGV | | | | | | | | | | | | |
| | (SEQ ID NO: 18) | | | | | | | | | | | | |
| RA-9 | KDLGVILLKE YNPG | — | — | — | — | — | Positive | — | — | Positive | Positive | — | — |
| | YIGPRA TKAQC | | | | | | | | | | | | |
| | (SEQ ID NO: 19) | | | | | | | | | | | | |
| RA-10 | TKAQCYDYIL SRLS | — | — | — | — | — | — | — | — | — | — | — | — |
| | EAIEVL PENRE | | | | | | | | | | | | |
| | (SEQ ID NO: 20) | | | | | | | | | | | | |
| RA-11 | PENRESVLYV SRDY | — | — | — | — | — | — | — | — | — | — | — | — |
| | AYALRA RIYLA | | | | | | | | | | | | |
| | (SEQ ID NO: 21) | | | | | | | | | | | | |
| RA-12 | RIYLALGEYG KAAA | — | — | — | — | — | — | — | — | — | — | — | — |
| | DAKMVV DKYPL | | | | | | | | | | | | |
| | (SEQ ID NO: 22) | | | | | | | | | | | | |
| RA-13 | DKYPLIGAAD ASEF | — | — | — | — | — | — | — | — | — | — | — | — |
| | ENIYRS DANNP | | | | | | | | | | | | |
| | (SEQ ID NO: 23) | | | | | | | | | | | | |
| RA-14 | DANNPEIIFR GFAS | — | — | — | — | — | — | — | — | — | — | — | — |
| | ATLGSF TATTL | | | | | | | | | | | | |
| | (SEQ ID NO: 24) | | | | | | | | | | | | |
| RA-15 | TATTLNGAAP AGKD | — | — | — | — | — | — | — | — | — | — | — | — |
| | IKYNPS AVPFQ | | | | | | | | | | | | |
| | (SEQ ID NO: 25) | | | | | | | | | | | | |
| RA-16 | AVPFQWVVDL YENE | — | — | — | — | — | — | — | — | — | — | — | — |
| | DFRKSV YIAKV | | | | | | | | | | | | |
| | (SEQ ID NO: 26) | | | | | | | | | | | | |
| RA-17 | YIAKVVKKDK GYLV | — | — | — | — | — | — | — | Positive | Positive | — | — | — |
| | NKFLED KAYRD | | | | | | | | | | | | |
| | (SEQ ID NO: 27) | | | | | | | | | | | | |
| RA-18 | KAYRDVQDKP NLKV | — | — | — | — | — | — | — | — | — | — | — | — |
| | GARYFS VAEVY | | | | | | | | | | | | |
| | (SEQ ID NO: 28) | | | | | | | | | | | | |
| RA-19 | VAEVYLILVE SALQ | — | — | — | — | — | — | Positive | — | — | — | — | — |
| | TGDTPT AEKYL | | | | | | | | | | | | |
| | (SEQ ID NO: 29) | | | | | | | | | | | | |
| RA-20 | AEKYLKALSK ARGA | — | — | — | — | — | — | — | — | — | — | — | — |
| | EVSVVN MEALQ | | | | | | | | | | | | |
| | (SEQ ID NO: 30) | | | | | | | | | | | | |
| RA-21 | MEALQAERTR ELIG | — | — | — | — | — | — | — | — | — | — | Positive | — |
| | EGSRLR DMVRW | | | | | | | | | | | | |
| | (SEQ ID NO: 31) | | | | | | | | | | | | |
| RA-22 | DMVRWSIPNN HDAF | — | — | — | — | — | — | — | — | — | — | — | — |
| | ETQPGL EGFAN | | | | | | | | | | | | |
| | (SEQ ID NO: 32) | | | | | | | | | | | | |
| RA-23 | EGFANTTPLK AQAP | — | — | — | — | — | — | — | — | — | — | — | — |
| | VGFYAY TWEFP | | | | | | | | | | | | |
| | (SEQ ID NO: 33) | | | | | | | | | | | | |
| RA-24 | TWEFPQRDRQ TNPQ | — | — | — | — | — | — | — | — | — | — | — | — |
| | LIKNWP \| | | | | | | | | | | | | |
| | (SEQ ID NO: 34) | | | | | | | | | | | | |

Table 18 shows the peptide library for RagB-2 (SEQ ID NO: 4), and the monoclonal antibody capture of antigenic targets, both by monoclonal antibodies directed at RagB-2 and monoclonal antibodies which were identified as reacting to other RagB subtypes. The ELISA results showed that peptide antigen targets were detected by 3 out of 6 hybridoma antibodies of RagB-2, e.g., monoclonal antibody 1A4-3-7-7, 1A6-3-3 and 1B3-1-5 react with RB-18, RB-21 and RB-7 peptides respectively (Table 18). Mouse serum containing polyclonal antibodies raised from vaccination of RagB-2 protein was named 2019-G65-1, which reacts with RB-17 peptide.

Monoclonal antibodies that cross-reacted with RagB-2 protein also found peptide targets in the RagB-2 peptide library. For example, 1D2-2-1-3 (specific to RagB-3 protein) binds to RB-117 peptide, and 10C3-7-8 (specific to RagB-4 protein) binds to RB-21 peptide.

TABLE 18
RagB-2 (SEQ ID NO: 4) Peptide Library Screening Targeted Antigen

| | | | | | | | | | Cross-Reaction | | |
|---|---|---|---|---|---|---|---|---|---|---|---|
| | | | | | | | | | antobodies | | |
| | | | | | | | | | raised from other | | |
| | | Antibodies to RagB-2 | | | | | | | subtype of RagB | | |
| | | 2019- | 1A3-4- | 1A4-3- | 1A5-4- | 1 A6- | 1A9-8- | 1B3- | 1D2-2- | 1B11- | 1C3- |
| | | G65-1 | 1-7 | 7-7 | 1-5 | 3-3 | 2-8 | 1-5 | 1-3 | 4-4 | 7-8 |
| Peptide's Name and | | Serum | Super- | Asci- | Asci- | Super- | Asci- | Asci- | Recom- | Recom- | Recom- |
| amino acid Sequence | | (P) | natant | tes | tes | natan | tes | tes | binant | binant | binant |
| RB-1 | CDLDRTPHNS | — | — | — | — | — | — | — | — | — | — |
| | DVQKPYEDMA | | | | | | | | | | |
| | TTVQY | | | | | | | | | | |
| | (SEQ ID NO: 35) | | | | | | | | | | |
| RB-2 | TTVQYRDGLY | — | — | — | — | — | — | — | — | — | — |
| | SVLRGAENAG | | | | | | | | | | |
| | RYTLS | | | | | | | | | | |
| | (SEQ ID NO: 36) | | | | | | | | | | |
| RB-3 | RYTLSEYMSD | — | — | — | — | — | — | — | — | — | — |
| | MYCVMQGDGG | | | | | | | | | | |
| | HATPY | | | | | | | | | | |
| | (SEQ ID NO: 37) | | | | | | | | | | |
| RB-4 | HATPYVTWTI | — | — | — | — | — | — | — | — | — | — |
| | PRIEIADHAS | | | | | | | | | | |
| | NYYFG | | | | | | | | | | |
| | (SEQ ID NO: 38) | | | | | | | | | | |
| RB-5 | NYYFGFNRLI | — | — | — | — | — | — | — | — | — | — |
| | QQANAFVGNV | | | | | | | | | | |
| | KLAIA | | | | | | | | | | |
| | (SEQ ID NO: 39) | | | | | | | | | | |
| RB-6 | KLAIANGVYK | — | — | — | — | — | — | — | — | — | — |
| | TEVDKTNAQI | | | | | | | | | | |
| | YLAEA | | | | | | | | | | |
| | (SEQ ID NO: 40) | | | | | | | | | | |
| RB-7 | YLAEAKTLQA | — | — | — | — | — | — | Posi- | — | — | — |
| | LALFRLMERF | | | | | | | tive | | | |
| | AYPYD | | | | | | | | | | |
| | (SEQ ID NO: 41) | | | | | | | | | | |
| RB-8 | AYPYDPNETT | — | — | — | — | — | — | — | — | — | — |
| | SPKNLGVVLI | | | | | | | | | | |
| | KEYDP | | | | | | | | | | |
| | (SEQ ID NO: 42) | | | | | | | | | | |
| RB-9 | KEYDPWAVGA | — | — | — | — | — | — | — | — | — | — |
| | RATQTETYSY | | | | | | | | | | |
| | IMSLL | | | | | | | | | | |
| | (SEQ ID NO: 43) | | | | | | | | | | |
| RB-10 | IMSLLDEAIS | — | — | — | — | — | — | — | — | — | — |
| | VLPETNANNM | | | | | | | | | | |
| | YVSRD | | | | | | | | | | |
| | (SEQ ID NO: 44) | | | | | | | | | | |
| RB-11 | YVSRDYALGL | — | — | — | — | — | — | — | — | — | — |
| | RARVHMAMDN | | | | | | | | | | |
| | YAEAA | | | | | | | | | | |
| | (SEQ ID NO: 45) | | | | | | | | | | |
| RB-12 | YAEAANDIRA | — | — | — | — | — | — | — | — | — | — |
| | FYKKYNLISA | | | | | | | | | | |
| | ANSDE | | | | | | | | | | |
| | (SEQ ID NO: 46) | | | | | | | | | | |
| RB-13 | ANSDEFEEAY | — | — | — | — | — | — | — | — | — | — |
| | RKMSSNPELI | | | | | | | | | | |
| | FRGYA | | | | | | | | | | |
| | (SEQ ID NO: 47) | | | | | | | | | | |
| RB-14 | FRGYASVTNG | — | — | — | — | — | — | — | — | — | — |
| | YLVYQDLMGA | | | | | | | | | | |
| | TASGT | | | | | | | | | | |
| | (SEQ ID NO: 48) | | | | | | | | | | |
| RB-15 | TASGTNVKYN | — | — | — | — | — | — | — | — | — | — |
| | PRVTPLQWVC | | | | | | | | | | |
| | DLYDA | | | | | | | | | | |
| | (SEQ ID NO: 49) | | | | | | | | | | |
| RB-16 | DLYDAADYRK | — | — | — | — | — | — | — | — | — | — |
| | KVYIVDKVNG | | | | | | | | | | |
| | DGGKG | | | | | | | | | | |
| | (SEQ ID NO: 50) | | | | | | | | | | |
| RB-17 | DGGKGYVVNK | Posi- | — | — | — | — | — | — | Posi- | — | — |
| | FLGDPELRED | tive | | | | | | | tive | | |
| | PKKEN | | | | | | | | | | |
| | (SEQ ID NO: 51) | | | | | | | | | | |
| RB-18 | PKKENFKTGC | — | — | Posi- | — | — | — | — | — | — | — |
| | RFFSLAEAYL | | | tive | | | | | | | |
| | ILAEA | | | | | | | | | | |
| | (SEQ ID NO: 52) | | | | | | | | | | |
| RB-19 | ILAEADIMTG | — | — | — | — | — | — | — | — | — | — |
| | NTAEAMEVLK | | | | | | | | | | |
| | ELSKS | | | | | | | | | | |
| | (SEQ ID NO: 53) | | | | | | | | | | |
| RB-20 | ELSKSRGAEV | — | — | — | — | — | — | — | — | — | — |
| | SGADYMQILK | | | | | | | | | | |
| | DERTR | | | | | | | | | | |
| | (SEQ ID NO: 54) | | | | | | | | | | |
| RB-21 | DERTREMIGE | — | — | — | — | Posi- | — | — | — | — | Posi- |
| | GSRLNDMIRW | | | | | tive | | | | | tive |
| | NMDLV | | | | | | | | | | |
| | (SEQ ID NO: 55) | | | | | | | | | | |
| RB-22 | NMDLVVSPVQ | — | — | — | — | — | — | — | — | — | — |
| | AVLHKIAVPT | | | | | | | | | | |
| | ILQTD | | | | | | | | | | |
| | (SEQ ID NO: 56) | | | | | | | | | | |
| RB-23 | ILQTDDPTRV | — | — | — | — | — | — | — | — | — | — |
| | PAGFYAFTWE | | | | | | | | | | |
| | IPNRD | | | | | | | | | | |
| | (SEQ ID NO: 57) | | | | | | | | | | |
| RB-24 | IPNRDLVVIP | — | — | — | — | — | — | — | — | — | — |
| | ELVRNWPKQ | | | | | | | | | | |
| | (SEQ ID NO: 58) | | | | | | | | | | |

TABLE 19
RagB-3 (SEQ ID NO: 6) Peptide Library Screening Targeted Antigen

| | | Monoclonal Antibodies specific to RagB-3 | | | | | | | | | | | |
|---|---|---|---|---|---|---|---|---|---|---|---|---|---|
| | | 1C10-1-2-3 | 1C10-8- | 1D9-6- | 1E4-6-1-7 | 1E6-3-8-6 | 1F10-5-2-6 | 1H11-2- | 2B2-8-5-5 | 2B7-4-6-7 | 2B8-1-8-4 | Super- | Super- |
| Peptide's Name and | | Super- | 7-3 | 8-6 | Super- | Super- | Super- | 2-4 | Super- | Super- | Super- | natant | natant |
| amino acid Sequence | | natant | Ascites | Ascites | natant | natant | natant | Ascites | natant | natant | natant | 2C1-4-6-2 | 2C4-6-1-4 |
| RC-1 | LNRDPEDKAK KEPFKTIAQA | — | — | — | — | — | — | — | — | — | Positive | — | — |
| | RQSRD (SEQ ID NO: 59) | | | | | | | | | | | | |
| RC-2 | RQSRDGLYSL LRESEQLAFH | — | — | — | — | — | — | — | — | — | Positive | — | — |
| | VYDEV (SEQ ID NO: 60) | | | | | | | | | | | | |
| RC-3 | VYDEVQSDMY TTTKNDGNQY | — | — | Positive- | — | — | — | — | — | — | Positive | — | — |
| | YPFVA (SEQ ID NO: 61) | | | weak | | | | | | | | | |
| RC-4 | YPFVAWQLGS METHGRADED | — | — | Positive | — | — | — | — | — | — | Positive | — | — |
| | QVYGI (SEQ ID NO: 62) | | | | | | | | | | | | |
| RC-5 | QVYGIAGYYF EYSRLIQQAN | — | — | — | — | — | — | — | — | — | Positive | — | — |
| | SYIED (SEQ ID NO: 63) | | | | | | | | | | | | |
| RC-6 | SYIEDMEKAL ANNTDMSIFI | — | — | — | — | — | — | — | — | — | — | — | — |
| | SSEEV (SEQ ID NO: 64) | | | | | | | | | | | | |
| RC-7 | SSEEVERAKR YLAEVRVVQA | — | — | — | — | — | — | Positive- | — | — | Positive- | — | — |
| | LANWR (SEQ ID NO: 65) | | | | | | | weak | | | weak | | |
| RC-8 | LANWRLMDRF AYKYDAASNQ | — | — | — | — | — | — | — | — | — | — | — | — |
| | SPQNL (SEQ ID NO: 66) | | | | | | | | | | | | |
| RC-9 | SPQNLGIVLI KEFNPAYIGP | Positive- | — | — | Positive | Positive | — | — | Positive- | Positive | Positive | Positive- | Positive |
| | RATQK (SEQ ID NO: 67) | weak | | | | | | | weak | | | weak | |
| RC-10 | RATQKECYDH ILSALEAAIT | Positive- | — | Positive- | — | — | Positive | Positive- | — | — | Positive | — | — |
| | VLPEK (SEQ ID NO: 68) | weak | | weak | | | | weak | | | | | |
| RC-11 | VLPEKNPDGI TYAGRDYAHA | Positive- | — | — | — | — | — | Positive- | — | — | Positive | — | — |
| | LRARV (SEQ ID NO: 69) | weak | | | | | | weak | | | | | |
| RC-12 | LRARVHLSMG NYADALADAK | Positive | — | Positive | — | — | Positive- | — | — | — | Positive | — | — |
| | QIVEK (SEQ ID NO: 70) | | | | | | weak | | | | | | |
| RC-13 | QIVEKYPLIN ANNAEDFAKI | Positive- | — | Positive | — | Positive | — | Positive | — | Positive | Positive | — | — |
| | YRSDA (SEQ ID NO: 71) | weak | | | | | | | | | | | |
| RC-14 | YRSDANNPEI VFRAFASPTT | Positive- | — | Positive | — | — | — | — | — | — | Positive | — | — |
| | GAVGA (SEQ ID NO: 72) | weak | | | | | | | | | | | |
| RC-15 | GAVGATSLNG ASVVNKKIKY | Positive- | — | — | — | — | — | — | — | — | Positive | — | — |
| | APFIV (SEQ ID NO: 73) | weak | | | | | | | | | | | |
| RC-16 | APFIVPLQWV VDLYEDADYR | Positive- | — | — | — | — | — | Positive- | — | — | Positive | — | — |
| | KSVYI (SEQ ID NO: 74) | weak | | | | | | weak | | | | | |
| RC-17 | KSVYIDKTVS NGSEKGYLVN | Positive- | — | Positive | — | — | — | Positive | — | — | Positive | Positive | — |
| | KFLED (SEQ ID NO: 75) | weak | | | | | | | | | | | |
| RC-18 | KFLEDPAYRE TADIPILKIG | Positive- | — | Positive | — | — | — | Positive | — | — | Positive | — | — |
| | VRMFS (SEQ ID NO: 76) | weak | | | | | | | | | | | |
| RC-19 | VRMFSIAEAY LMVAECAHMT | Positive- | — | Positive | — | — | — | Positive | — | — | Positive | — | — |
| | GDDAT (SEQ ID NO: 77) | weak | | | | | | | | | | | |
| RC-20 | GDDATAIAYL KKLSAARGGN | Positive- | — | Positive | — | — | — | Positive- | — | — | Positive | — | — |
| | IDTGD (SEQ ID NO: 78) | weak | | | | | | weak | | | | | |
| RC-21 | IDTGDVMKAI QEERTRELIG | Positive- | — | Positive | — | — | — | Positive- | — | — | Positive | — | — |
| | EGARL (SEQ ID NO: 79) | weak | | | | | | weak | | | | | |
| RC-22 | EGARLRDMIR WNLPNIDKTE | Positive- | — | Positive | — | — | — | Positive | — | — | Positive | — | — |
| | IQPAL (SEQ ID NO: 80) | weak | | | | | | | | | | | |
| RC-23 | IQPALTGYAR EIVLEQPVPA | Positive- | — | Positive | — | — | — | Positive | — | — | Positive | — | — |
| | GHYAF (SEQ ID NO: 81) | weak | | | | | | | | | | | |
| RC-24 | GHYAFTWEFP NRDRQVNPHL | Positive- | — | Positive | — | — | Positive- | Positive | — | — | Positive | — | — |
| | IKNW (SEQ ID NO: 82) | weak | | | | | weak | | | | | | |

TABLE 20
RagB-3 (SEQ ID NO: 6) Peptide Library Screening Targeted Antigen

| | | Polyclonal Antibodies to | | | | | Monoclonal Antibodies | | | | Cross-Reaction antobodies raised | | |
|---|---|---|---|---|---|---|---|---|---|---|---|---|---|
| | | RagB-3 and other RagB subtype | | | | | specific to RagB-3 | | | | from other subtype of RagB | | |
| | | 2019- | 2019G160- | 2019-G160- | 2019- | | 2C5-4-6-6 | 2C8-7- | 2E6-4-4-8 | 1D2-2-1-3 | 1A4-3- | 1B11-4-4 | 1C3-7-8 |
| Peptide's Name and | | G75-2 | 2 | 4(M) | G250-3 | PBS | Super- | 6-6 | Super- | Recom- | 7-7 | Recom- | Recom- |
| amino acid Sequence | | Serum | Serum | Serum | Serum | Control | natant | Ascites | natant | binant | Ascites | binant | binant |
| RC-1 | LNRDPEDKAK KEPFKTIAQA | — | — | — | — | — | — | — | Positive- | — | — | — | — |
| | RQSRD (SEQ ID NO: 59) | | | | | | | | weak | | | | |
| RC-2 | RQSRDGLYSL LRESEQLAFH | — | — | — | — | — | — | — | Positive- | — | — | — | — |
| | VYDEV (SEQ ID NO: 60) | | | | | | | | weak | | | | |
| RC-3 | VYDEVQSDMY TTTKNDGNQY | — | — | — | — | — | — | — | Positive- | — | — | — | — |
| | YPFVA (SEQ ID NO: 61) | | | | | | | | weak | | | | |
| RC-4 | YPFVAWQLGS METHGRADED | — | — | — | — | — | — | — | Positive- | — | — | — | — |
| | QVYGI (SEQ ID NO: 62) | | | | | | | | weak | | | | |
| RC-5 | QVYGIAGYYF EYSRLIQQAN | — | — | — | — | — | — | — | Positive- | — | — | — | — |
| | SYIED (SEQ ID NO: 63) | | | | | | | | weak | | | | |
| RC-6 | SYIEDMEKAL ANNTDMSIFI | — | — | — | — | — | — | — | — | — | — | — | — |
| | SSEEV (SEQ ID NO: 64) | | | | | | | | | | | | |
| RC-7 | SSEEVERAKR YLAEVRVVQA | — | — | — | — | — | — | — | Positive- | — | — | — | — |
| | LANWR (SEQ ID NO: 65) | | | | | | | | weak | | | | |
| RC-8 | LANWRLMDRF AYKYDAASNQ | — | — | — | — | — | — | — | Positive- | — | — | — | — |
| | SPQNL (SEQ ID NO: 66) | | | | | | | | weak | | | | |
| RC-9 | SPQNLGIVLI KEFNPAYIGP | — | — | — | — | — | — | — | Positive- | — | — | — | — |
| | RATQK (SEQ ID NO: 67) | | | | | | | | weak | | | | |
| RC-10 | RATQKECYDH ILSALEAAIT | — | — | — | — | — | Positive- | — | Positive- | — | — | — | — |
| | VLPEK (SEQ ID NO: 68) | | | | | | weak | | weak | | | | |
| RC-11 | VLPEKNPDGI TYAGRDYAHA | — | — | — | — | — | Positive- | — | Positive- | — | — | — | — |
| | LRARV (SEQ ID NO: 69) | | | | | | weak | | weak | | | | |
| RC-12 | LRARVHLSMG NYADALADAK | — | — | — | — | — | Positive- | — | Positive | — | — | — | — |
| | QIVEK (SEQ ID NO: 70) | | | | | | weak | | | | | | |
| RC-13 | QIVEKYPLIN ANNAEDFAKI | — | — | — | — | — | Positive | — | Positive- | — | — | — | — |
| | YRSDA (SEQ ID NO: 71) | | | | | | | | weak | | | | |
| RC-14 | YRSDANNPEI VFRAFASPTT | — | — | — | — | — | — | — | Positive- | — | — | — | — |
| | GAVGA (SEQ ID NO: 72) | | | | | | | | weak | | | | |
| RC-15 | GAVGATSLNG ASVVNKKIKY | — | — | — | — | — | — | — | Positive- | — | — | — | — |
| | APFIV (SEQ ID NO: 73) | | | | | | | | weak | | | | |
| RC-16 | APFIVPLQWV VDLYEDADYR | — | — | — | — | — | — | — | Positive- | — | — | — | — |
| | KSVYI (SEQ ID NO: 74) | | | | | | | | weak | | | | |
| RC-17 | KSVYIDKTVS NGSEKGYLVN | — | — | — | — | — | — | — | Positive- | Positive | — | — | — |
| | KFLED (SEQ ID NO: 75) | | | | | | | | weak | | | | |
| RC-18 | KFLEDPAYRE TADIPILKIG | — | — | — | — | — | Positive- | — | Positive | — | — | — | — |
| | VRMFS (SEQ ID NO: 76) | | | | | | weak | | | | | | |
| RC-19 | VRMFSIAEAY LMVAECAHMT | — | — | — | — | — | — | — | Positive- | — | — | — | — |
| | GDDAT (SEQ ID NO: 77) | | | | | | | | weak | | | | |
| RC-20 | GDDATAIAYL KKLSAARGGN | — | — | — | — | — | — | — | Positive- | — | — | — | — |
| | IDTGD (SEQ ID NO: 78) | | | | | | | | weak | | | | |
| RC-21 | IDTGDVMKAI QEERTRELIG | — | — | — | — | — | — | — | Positive- | — | — | Positive | — |
| | EGARL (SEQ ID NO: 79) | | | | | | | | weak | | | | |
| RC-22 | EGARLRDMIR WNLPNIDKTE | — | — | — | — | — | — | — | Positive- | — | — | — | Positive |
| | IQPAL (SEQ ID NO: 80) | | | | | | | | weak | | | | |
| RC-23 | IQPALTGYAR EIVLEQPVPA | — | — | — | — | — | Positive- | — | Positive- | — | — | — | — |
| | GHYAF (SEQ ID NO: 81) | | | | | | weak | | weak | | | | |
| RC-24 | GHYAFTWEFP NRDRQVNPHL | — | — | — | — | — | — | Positive- | Positive- | — | — | — | — |
| | IKNW (SEQ ID NO: 82) | | | | | | | weak | weak | | | | |

Table 19 and Table 20 show the peptide library for RagB-3 (SEQ ID NO: 6), and the monoclonal antibody capture of antigenic targets, both by monoclonal antibodies directed at RagB-3 and monoclonal antibodies which were identified as reacting to other RagB subtypes. The ELISA results showed that peptide antigen targets were detected by almost all tested hybridoma antibodies of RagB-3, with 15 out of 16 hybridoma antibodies showing reactions with RagB-3 library peptides.

Surprisingly, the RagB-3 group data also show that several monoclonal antibodies react with multiple peptides at different positions. In addition, four different mouse polyclonal antibodies raised from a single protein vaccine (Table 20, 2019-G75-2) and multivalent RagB vaccine immunization (Table 20, 2019-G160-2, 2019-G160-4 and 2019-G250-3), were all unsuccessful at detecting a clear peptide target in the library. The 1D2-2-1-3 recombinant plasmid antibody showed a highly specific reaction with the RC-17 peptide, but no reaction with other peptide fragments was observed.

Certain peptide targets in the RagB-3 peptide library were also identified with the tested monoclonal antibodies. For example, it was found that the 1B11-4-4 antibody (specific to RagB-4 protein) reacted with RC-21 peptide, and 1C3-7-8 antibody (specific to RagB-4 protein) reacted with RC-22 peptide.

TABLE 21
RagB-4 (SEQ ID NO: 8) Peptide Library Screening Targeted Antigen

| | | Antibodies to RagB-4 | | | | | | | | Possible Cross Reaction Check | | | |
|---|---|---|---|---|---|---|---|---|---|---|---|---|---|
| | | G90 | | | 1B11-4-4 | 1C3-7-8 | D6-3-8 | 1E2-3-7 | 1D10-2-8 | 1A4-3- | 1D2-2-1-3 | 1E4-6-1-7 | 1H11-2-2-4 |
| Peptide's Name and | | Serum | 1B6-1-8 | 1B10-6-8 | Recom- | Recom- | Super- | Super- | Super- | 7-7 | Recom- | Super- | Super- |
| amino acid Sequence | | (P) | Ascites | Ascites | binant | binant | natant | natant | natant | Ascites | binant | natant | natant |
| RD-1 | MDPDGSDEQK DHFASFVETK | Positive | — | — | — | — | — | — | — | — | — | — | — |
| | HFRDG (SEQ ID NO: 83) | | | | | | | | | | | | |
| RD-2 | HFRDGLYATL RSTENPTRFV | — | — | — | — | — | — | — | — | — | — | — | — |
| | WQDLQ (SEQ ID NO: 84) | | | | | | | | | | | | |
| RD-3 | WQDLQSDMYA VTTNDGNTSS | — | — | — | — | — | — | — | — | — | — | — | — |
| | RFITW (SEQ ID NO: 85) | | | | | | | | | | | | |
| RD-4 | RFITWSLGAL ESSGEIASYY | — | — | — | — | — | — | — | — | — | — | — | — |
| | LAYYS (SEQ ID NO: 86) | | | | | | | | | | | | |
| RD-5 | LAYYSLLQRA NYFVTRIERS | — | — | Positive | — | — | — | — | — | — | — | — | — |
| | MELNL (SEQ ID NO: 87) | | | | | | | | | | | | |
| RD-6 | MELNLYLEKE LKDVKIFQAE | — | — | — | — | — | — | — | — | — | — | — | — |
| | GKTLQ (SEQ ID NO: 88) | | | | | | | | | | | | |
| RD-7 | GKTLQALALS RLMERFAYKY | — | — | — | — | — | — | — | — | — | — | — | — |
| | DPAAT (SEQ ID NO: 89) | | | | | | | | | | | | |
| RD-8 | DPAATTHPYD LGIVLVKDYN | — | — | — | — | — | — | — | — | — | — | — | — |
| | PMIAA (SEQ ID NO: 90) | | | | | | | | | | | | |
| RD-9 | PMIAAPRNTQ KECYDYILEC | — | — | — | — | — | — | — | — | — | — | — | — |
| | LNQAI (SEQ ID NO: 91) | | | | | | | | | | | | |
| RD-10 | LNQAIDVLPN KSNEGNIRVS | — | — | — | — | — | — | — | — | — | — | — | — |
| | KHYAH (SEQ ID NO: 92) | | | | | | | | | | | | |
| RD-11 | KHYAHALRAR VNFAMGNYDA | — | — | — | — | — | — | — | — | — | — | — | — |
| | AKEDA (SEQ ID NO: 93) | | | | | | | | | | | | |
| RD-12 | AKEDAKVLVD NYPLIDVTTA | — | — | — | — | — | — | — | — | — | — | — | — |
| | KKFAE (SEQ ID NO: 94) | | | | | | | | | | | | |
| RD-13 | KKFAEVYRDD ANNPEIVFRA | — | — | — | — | — | — | — | — | — | — | — | — |
| | FASGT (SEQ ID NO: 95) | | | | | | | | | | | | |
| RD-14 | FASGTIGTVA ETTLSGFLWH | — | — | — | — | — | — | — | — | — | — | — | — |
| | SGAQL (SEQ ID NO: 96) | | | | | | | | | | | | |
| RD-15 | SGAQLVVSSP ISAPFQWVVD | — | — | — | — | — | — | — | — | — | — | — | — |
| | LYDDT (SEQ ID NO: 97) | | | | | | | | | | | | |
| RD-16 | LYDDTDYRKS CYITKDFYVI | — | — | — | — | — | — | — | — | — | — | — | — |
| | GGGVD (SEQ ID NO: 98) | | | | | | | | | | | | |
| RD-17 | GGGVDKGYVV GKYLGNPAYQ | — | — | — | — | — | — | — | Positive | — | — | — | — |
| | SNPNV (SEQ ID NO: 99) | | | | | | | | | | | | |
| RD-18 | SNPNVPDFKV TSRFFSVAEA | — | — | — | — | — | — | — | — | — | — | — | — |
| | YLIMA (SEQ ID NO: 100) | | | | | | | | | | | | |
| RD-19 | YLIMAESMAK SGDAAGAKDL | — | — | — | — | — | — | — | — | — | — | — | — |
| | LKTLC (SEQ ID NO: 101) | | | | | | | | | | | | |
| RD-20 | LKTLCEKRGG QLEDGDIMDL | — | — | — | — | — | — | — | — | — | — | — | — |
| | VMAER (SEQ ID NO: 102) | | | | | | | | | | | | |
| RD-21 | VMAERTRELI GEGSRLNDMI | — | — | — | Positive | Positive | — | — | — | — | — | — | — |
| | RWNLP (SEQ ID NO: 103) | | | | | | | | | | | | |
| RD-22 | RWNLPNNHDD MENQPVFLQI | — | Positive | Positive | — | — | Positive | — | — | — | — | — | — |
| | GLAKA (SEQ ID NO: 104) | | | | | | | | | | | | |
| RD-23 | GLAKADKLKQ PVPAGHYAFT | — | — | — | — | — | — | — | — | — | — | — | — |
| | WEFPV (SEQ ID NO: 105) | | | | | | | | | | | | |
| RD-24 | WEFPVRDRQV NPQIIKNWPN | — | — | — | — | — | — | — | — | — | — | — | — |
| | (SEQ ID NO: 106) | | | | | | | | | | | | |

Table 21 shows the peptide library for RagB-4 (SEQ ID NO: 8), and the monoclonal antibody capture of antigenic targets, both by monoclonal antibodies directed at RagB-4 and monoclonal antibodies which were identified as reacting to other RagB subtypes. The ELISA results showed that peptide antigen targets were detected by hybridoma antibodies of RagB-4, 6 out of 7 hybridoma antibodies showed reactions with RagB-4 library peptides. Two RagB-3 monoclonal antibodies 1E4-6-1-7 and 1H11-2-2-4 cross-reacted with RagB-3 protein, but no reaction between these antibodies and cross-reaction with any RagB-4 peptides were found. Mouse serum containing polyclonal antibodies raised from vaccination with RagB-4 protein was named G90 serum (P), which reacted with RD-1 peptide.

In the antigen target capture study of Cra4S1 monoclonal antibodies, the culture supernatant of more than 40 hybridoma-positive cell lines were tested for antigen target screening. Although these antibodies reacted with the recombinant protein with high titers, they did not react with artificial synthetic peptide antigens created in the Cra4S1 peptide library. Only a few clones showed weak antigen-antibody reactions. Table 16 provides two sets of peptide libraries for Cra4S1. After adjusting the length and position of the peptide chain, no antigen-antibody binding reaction was detected

As previously noted, several monoclonal antibodies showed cross-reactivity to another RagB protein, and to fragments from the peptide library of another RagB protein. The important cross-antigenic targets are provided as follows:

### 1. Peptide Binding Site of Monoclonal Antibody RagB-2-1A4-3-7-7:

RA-19:
(SEQ ID NO: 29)
VAEVYLILVESALQTGDTPTAEKYL;
and/or
RB-18:
(SEQ ID NO: 52)
PKKENFKTGCRFFSLAEAYLILAEA

Monoclonal antibody 1A4-3-7-7, which is specific to RagB-2 protein, reacted with RA-19 and RB-18 peptides. The underlined amino acids are completely identical parts of the two sequences, therefore, the specific antigen-binding site of the RagB-2-1A4-3-7-7 monoclonal antibody is to these highlighted epitopes located in RA-19 (SEQ ID NO:29) and/or in RB-18 (SEQ ID NO:52).

### 2. Peptide Binding Site of Monoclonal Antibody RagB-3-1D2-2-1-3 (Part 1):

RA-17&18:
(SEQ ID NO: 123)
YIAKVVKKDKGYLVNKFLEDKAYRDVQDKPNLKVGARYFSVAEVY,
and/or
RC-17&18:
(SEQ ID NO: 124)
KSVYIDKTVSNGSEKGYLVNKFLEDPAYRETADIPILKIGVRMFS

### Peptide Binding Sites of Monoclonal Antibody RagB-3-1D2-2-1-3 (Part 2):

RB-17:
(SEQ ID NO: 51)
DGGKGYVVNKFLGDPELREDPKKEN;
and/or
RC-17&18:
(SEQ ID NO: 124)
KSVYIDKTVSNGSEKGYLVNKFLEDPAYRETADIPILKIGVRMFS

The underlined amino acids are completely identical of two sequences respectively, therefore, the specific antigen-binding site of the RagB-3-1D2-2-1-3 monoclonal antibody is in RA-17&18 (SEQ ID NO: 123), and/or RB-17 (SEQ ID NO: 51), and/or RC-17&18 (SEQ ID NO: 124). It showed that the monoclonal antibody RagB-3-1D2-2-1-3 has cross reactivity to two other RagB subtypes, but antibody-antigen binding site epitopes and lengths are not the same.

### 3. Peptide Binding Site of Monoclonal Antibody RagB-4-1B11-4-4 (Part 1)

RA-21:
(SEQ ID NO: 31)
MEALQAERTRELIGEGSRLR DMVRW;
and/or
RD-21:
(SEQ ID NO: 103)
VMAERTRELIGEGSRLNDMI RWNLP

### Peptide Binding Site of Monoclonal Antibody RagB-4-1B11-4-4 (Part 2)

RB-21:
(SEQ ID NO: 55)
DERTREMIGEGSRLNDMIRWNMDLV,
and/or
RD-21:
(SEQ ID NO: 103)
VMAERTRELIGEGSRLNDMIRWNLP

### Peptide Binding Site of Monoclonal Antibody RagB-4-1B11-4-4 (Part 3)

RC-21:
(SEQ ID NO: 79)
IDTGDVMKAIQEERTRELIGEGARL,
and/or
RD-21:
(SEQ ID NO: 103)
VMAERTRELIGEGSRLNDMIRWNLP

The underlined amino acids are completely identical of two sequences respectively, therefore, the specific antigen-binding site of the RagB-4-1B11-4-4 monoclonal antibody is in RA-21 (SEQ ID NO: 31), and/or RB-21 (SEQ ID NO: 55), and/or RC-21 (SEQ ID NO: 79) and RD-21 (SEQ ID NO: 103). Monoclonal antibody RagB-4-1B11-4-4 showed cross reactivity to three other RagB subtypes, but antibody-antigen binding site epitopes and lengths are not the same.

### 4. Pe Tide Binding Site of Monoclonal Antibody RagB-4-1C3-7-8 Art 1

RA-21&22:
(SEQ ID NO: 125)
MEALQAERTRELIGEGSRLRDMVRWSIPNNHDAFETQPGLEGFAN,
and/or
RD-21&22:
(SEQ ID NO: 126)
VMAERTRELIGEGSRLNDMIRWNLPNNHDDMENQPVFLQIGLA

### Peptide Binding Site of Monoclonal Antibody RagB-4-1C3-7-8 (Part 2)

RB-21:
(SEQ ID NO: 55)
DERTREMIGEGSRLNDMIRWNMDLV;
and/or
RD-21:
(SEQ ID NO: 103)
VMAERTRELIGEGSRLNDMIRWNLP

### Peptide Binding Site of Monoclonal Antibody RagB-4-1C3-7-8 (Part 3)

RC-22:
(SEQ ID NO: 80)
EGARLRDMIRWNLPNIDKTEIQPAL;
and/or
RD-21&22:
(SEQ ID NO: 126)
VMAERTRELIGEGSRLNDMIRWNLPNNHDDMENQPVFLQIGLAKA

The underlined amino acids are completely identical of the two sequences respectively, therefore, the specific antigen-binding site of the RagB-4-1C3-7-8 monoclonal antibody is in RA-21&22 (SEQ ID NO: 125), and/or RB-21 (SEQ ID NO: 55), and/or RC-22 (SEQ ID NO: 80) and RD-21&22 (SEQ ID NO: 126). Monoclonal antibody RagB-4-1C3-7-8 showed cross reactivity to three other RagB subtypes, but antibody-antigen binding site epitopes and lengths are not the same.

The discovery of polyclonal antibodies that cross react with different subtypes of RagB proteins and the discovery of monoclonal antibodies with multiple antigenic targets both are surprised and unexpected. Two monoclonal antibodies with the property of multiple cross-reactivity, 1B11-4-4 (SEQ ID NO: 148-149 and SEQ ID NO: 153-154) and 1C3-7-8 (SEQ ID NO: 158-159 and SEQ ID NO: 163-164), were obtained from RagB-4 immunization, and the data confirmed that their antigenic targets were in the region of RD-21 (SEQ ID NO:103) and RD-21 &22 (SEQ ID NO:126). Even though their antigenic target locations are close to each other, but each has its own unique position and sequence. Importantly, they both have common antigens with the antigenic determinants in different positions of RagB-1, RagB-2 and RagB-3 proteins.

In summary, the antigen-antibody reactions of monoclonal antibodies with RagB peptides confirm the antigenicity of certain fragments of the RagB peptides, providing a molecular basis for developing antibodies and vaccine thereof, that are capable of reacts with those antigenicity fragments of the RagB peptides so as to defeat bacterial infections caused by P. gingivalis. Two monoclonal antibodies, 1B11-4-4 (SEQ ID NO: 148-149 and SEQ ID NO: 153-154) and 1C3-7-8 (SEQ ID NO: 158-159 and SEQ ID NO: 163-164), have their own antigenic targets within the closely adjacent RD-21 (SEQ ID NO:103) and RD-21 &22 (SEQ ID NO:126) peptide fragments, and they both recognize antigens from the four different RagB subtypes. The adjacent antigenic targets represent ideal conditions for mixture of monoclonal antibodies and preparing bispecific antibodies.

### Example 5

### Heterophile Antigen and Pathogenic Mechanisms of P. Gingivalis

In recent years, both clinical studies and statistical surveys have shown that periodontal disease is associated with several systemic diseases, especially chronic diseases and tumors. Although there are many researches on P. gingivalis, how these distinct and wide-ranging diseases relate to one another remains unknown.

In Example 4, when monoclonal antibodies against the Cra4S1 protein were screened, although it was shown the antibody reacted strongly with the full-length recombinant protein of Cra4S1, no specific antigenic fragment target of this protein was identified. The study with the anti-Cra4S1 polyclonal antibody serum also failed to reveal a dominant peptide target in the Cra4S1 peptide library. These data indicate that there is likely no significant dominant antigenic fragment in Cra4S1, or that the antigenicity of Cra4S1 is diffuse and presents only through its three-dimensional spatial structure. Also, in Example 4, with the monoclonal antibody of RagB-3 protein, multiple targeted antigenic peptide sequence fragments were identified in the RagB-3 peptide library. However, the study with the polyclonal antibody serum failed to reveal a dominant peptide fragment and/or epitope of the RagB-3 in the peptide library. Nevertheless, both RagB-1 and RagB-4 protein immunized animals, and the polyclonal antibodies produced, were able to identify antigenic peptide fragments/targets at N-terminus of each peptide RA-1 and RD-1 respectively, and RagB-2 polyclonal antibody was able to react with peptide RB-17. Overall, these results suggest that the antigenic features of different outer membrane protein subtypes of P. gingivalis are different.

Reviewing studies on the Cra4S1 recombinant protein, it was shown in Example 2 that sera from animals following P. gingivalis challenge did not react with Cra4S1 but reacted with RagB, suggesting that the antigenicity of the Cra4S1 protein may not be dominant. The data in Example 2 also showed that, when RagB protein was used to immunize pre-infected animals, serum antibodies reacted with Cra4S1, but not with negative control GST protein, suggesting that RagB antibodies may cross react with the Cra4S1 protein. In certain embodiment, it was found that RagB-4-1B11-4-4 monoclonal antibody did cross react with Cra4S1.

Surprisingly, when human serum samples were used for antibodies to P. gingivalis, antibodies to both RagB and Cra4S1 were present in a considerable proportion of the specimens. Specifically, the human serum samples collected in this Example included healthy people, cardiovascular patients, and geriatric clinic patients. Table 22, Table 26, and Table 29 list the ELISA results of these human serum samples, detecting antibodies to P. gingivalis outer membrane protein. The design of the ELISA plate antigen coating is the same as that described in Example 2, the antigen layout design is briefly described as follows: the 1st and 7th columns were coated with RagB-1 recombinant protein, the 2nd and 8th columns were coated with RagB-2, and the 3rd and 9th columns were coated RagB-3, the 4th and 10th columns were coated with RagB-4, the 5th and 11th columns were coated with Cra4S1. The 6th and 12th columns were coated with GST recombinant protein as a negative irrelevant protein control.

The dilution of the first set of these human serum samples (healthy subjects) was 1:300, and each serum sample tested with 6-well antigen proteins, including RagB-1, RagB-2, RagB-3, RagB-4, Cra4S1 and GST, and the secondary antibody was alkaline phosphatase-conjugated with mouse anti-human IgG for human samples, and goat anti-mouse IgG for controls. Each ELISA plate was equipped with a PBS negative control, and the positive control (labelled M-PC) is from sera of immunized mice which had vaccination with a pentavalent protein, the vaccine components included RagB-1, RagB-2, RagB-3, RagB-4 and Cra4S1.

Since it was the first time to use human serum to specifically detect P. gingivalis outer membrane protein antibodies, the mean OD value of the lowest quintile (lowest 20%) of the total sample was pre-specified as negative, and the cut-off value was tentatively set to 2.5 times this value. If the OD value was more than 2.5 times the negative value, it was considered as positive. Table 22 shows the ELISA results of serum samples from 51 healthy subjects. Table 23 shows the lowest OD values for each group of detected proteins (20% of 51) from 10 subjects. Table 23 also lists the number of samples whose OD value is 2.5 times higher than the negative OD value (deemed the positive results), and the data of suspected positive subjects whose OD value is between 1.8 and 2.5 times the negative value. An OD value of more than four times (4×) the negative value is deemed a strong positive result and reflects a quantitively higher protein titer.

The first set of human serum samples consisted of 51 healthy individuals (blood tests from young adults, numbered from H1 to H51). The positive rate of RagB-1 antibody test was 11.8% (6/51), of which the strong positive rate was 2% (1/51), and its OD value was more than 4 times that of the negative; the positive rate of RagB-2 antibody test was 0% (0/51); the positive rate of RagB-3 antibody test was 2% (1/51); the positive rate of RagB-4 antibody was 31.3% (16/51), of which the strong positive rate was 13.7% (7/51). The rate of weakly positive results (1.8-2.5 times the negative serum OD value) was 29.4% (15/51), indicating that RagB-4 antibody-positive samples accounted for a relatively large proportion of the total samples (15/51 weak positive, 9/51 positive and 7/51 strong positive). The positive rate of Cra4S1 antibody was 9.8% (5/51), and the positive rate of irrelevant protein GST antibody was 0% (0/51).

The detection rate of RagB-1 and Cra4S1 antibodies was lower than that of RagB-4, and some RagB-1 antibodies were positively associated with RagB-4 antibodies, that is, when RagB-4 antibodies were detected, the higher the OD value, the higher the OD value for RagB-1 antibody. The detection rates of RagB-2, RagB-3 and GST antibodies were at a very low level (FIGS. 12A-12B, Table 22, Table 23, and Table 24). In these people, is it possible that RagB-2 and RagB-3 subtypes are less highly antigenic, and thus do not result in a significant antibody response?

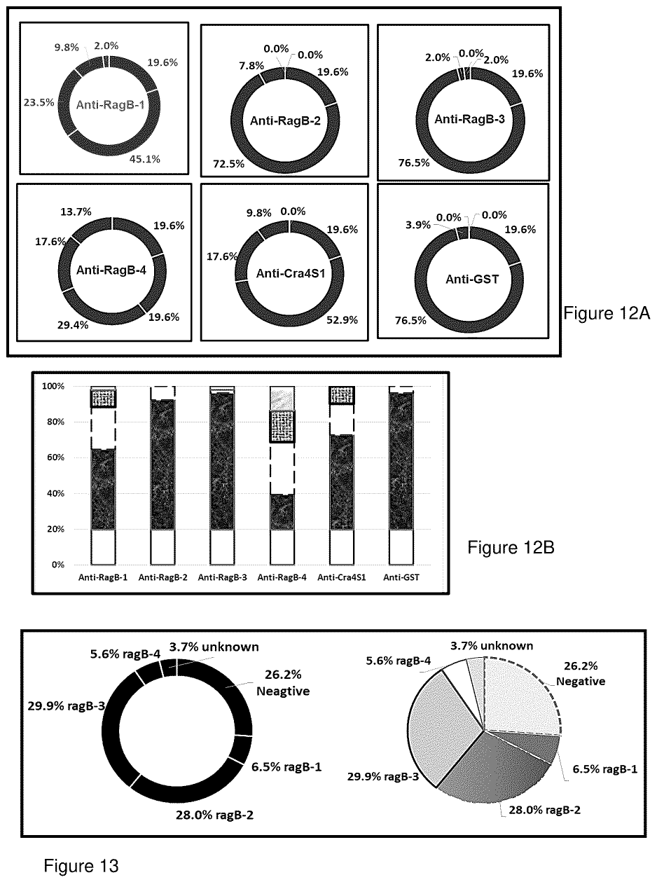

Conversely, the ragB-2 and ragB-3 alleles detected by P. gingivalis PCR nucleic acid represented the most prevalent group in the studied periodontal patient population. Spanning the years 2002-2004, an etiological study of patients with periodontal disease attending the outpatient department of the Dental School of the Royal London Hospital UK was performed. A total of 107 samples were obtained, and the PCR method was used to detect P. gingivalis and its subtypes from the gingival crevicular fluid samples of patients with periodontal disease. The detailed method is described in patent application PCT/GB2005/001976, which is incorporated by reference herewith. Briefly, whole DNA was extracted from the gingival crevicular fluid samples, and 16s RNA of P. gingivalis was identified by PCR. Four pairs of ragB primers, which were specifically designed for each RagB outer membrane protein subtype of P. gingivalis, were used to detect ragB alleles. The results showed that RagB-2 and RagB-3 subtypes together accounted for about 58% of total samples (FIG. 13, Table 25). The results of these clinical specimens showed that the RagB-2 and RagB-3 P. gingivalis subtypes were relatively high in the periodontal disease population geographically in local east London.

Virulence studies of different bacterial strains using animal models of soft tissue destruction, showed that no outer membrane protein subtype is clearly shown more or less virulent than another. The mechanisms by which bacterial virulence and immune responses cause local and systemic damage to organisms are a mystery and an urgently desired milestone.

The same ELISA method was used for the detection of human serum antibodies in cardiovascular and geriatric patients. Table 26 shows the ELISA results of serological detection in geriatric patients. Briefly, antigen coating and assay procedure were the same as described above, for plate 20-05-P5, 20-05-P6 and 20-05-P7, the primary antibody (patient serum) was in 1:1000 dilution, plate 20-05-P8 primary antibody (patient serum) 1:500 dilution. To avoid false positive readings, the sample dilutions were increased from the 1:300 to 1:500 and 1:1000.

TABLE 22
Investigation of P. gingivalis Antibodies in Healthy Human Serum

| | Specimen | | | | | | | | | | | | | Specimen |
|---|---|---|---|---|---|---|---|---|---|---|---|---|---|---|
| | number | RagB-1 | RagB-2 | RagB-3 | RagB-4 | Cra4S1 | GST | RagB-1 | RagB-2 | RagB-3 | RagB-4 | Cra4S1 | GST | number |
| 20-04-P1 | | | | | | | | | | | | | | |
| A | PBS | 0.034 | 0.033 | 0.033 | 0.039 | 0.036 | 0.033 | 0.152 | 0.099 | 0.195 | 0.653 | 0.258 | 0.055 | H7 |
| B | M-PC | 0.201 | 0.471 | 0.299 | 0.371 | 1.279 | 0.034 | 0.124 | 0.085 | 0.053 | 0.258 | 0.267 | 0.052 | H8 |
| C | H1 | 0.126 | 0.067 | 0.056 | 0.25 | 0.341 | 0.057 | 0.078 | 0.058 | 0.047 | 0.098 | 0.17 | 0.044 | H9 |
| D | H2 | 0.108 | 0.063 | 0.052 | 0.166 | 0.212 | 0.051 | 0.16 | 0.078 | 0.056 | 0.194 | 0.289 | 0.056 | H10 |
| E | H3 | 0.142 | 0.072 | 0.057 | 0.512 | 0.315 | 0.055 | 0.125 | 0.068 | 0.059 | 0.19 | 0.261 | 0.056 | H11 |
| F | H4 | 0.317 | 0.112 | 0.069 | 0.767 | 0.517 | 0.071 | 0.181 | 0.068 | 0.056 | 0.284 | 0.424 | 0.049 | H12 |
| G | H5 | 0.214 | 0.079 | 0.051 | 0.233 | 0.312 | 0.05 | 0.085 | 0.049 | 0.041 | 0.116 | 0.124 | 0.035 | H13 |
| H | H6 | 0.211 | 0.073 | 0.052 | 0.32 | 0.423 | 0.054 | 0.132 | 0.057 | 0.049 | 0.232 | 0.316 | 0.051 | H14 |
| 20-04-P2 | | | | | | | | | | | | | | |
| A | PBS | 0.033 | 0.032 | 0.032 | 0.034 | 0.034 | 0.034 | 0.125 | 0.073 | 0.057 | 0.32 | 0.317 | 0.049 | H21 |
| B | M-PC | 0.191 | 0.472 | 0.304 | 0.397 | 1.334 | 0.034 | 0.134 | 0.082 | 0.058 | 0.415 | 0.37 | 0.063 | H22 |
| C | H15 | 0.073 | 0.049 | 0.044 | 0.146 | 0.231 | 0.043 | 0.071 | 0.046 | 0.044 | 0.071 | 0.189 | 0.038 | H23 |
| D | H16 | 0.153 | 0.075 | 0.055 | 0.28 | 0.296 | 0.054 | 0.094 | 0.056 | 0.049 | 0.172 | 0.224 | 0.051 | H24 |
| E | H17 | 0.16 | 0.07 | 0.054 | 0.28 | 0.396 | 0.049 | 0.169 | 0.076 | 0.053 | 0.501 | 0.46 | 0.065 | H25 |
| F | H18 | 0.11 | 0.054 | 0.044 | 0.134 | 0.266 | 0.044 | 0.269 | 0.1 | 0.069 | 0.824 | 0.464 | 0.046 | H26 |
| G | H19 | 0.175 | 0.064 | 0.068 | 0.234 | 0.397 | 0.054 | 0.105 | 0.056 | 0.043 | 0.095 | 0.212 | 0.038 | H27 |
| H | H20 | 0.096 | 0.05 | 0.037 | 0.151 | 0.233 | 0.039 | 0.147 | 0.062 | 0.051 | 0.376 | 0.346 | 0.048 | H28 |
| 20-04-P3 | | | | | | | | | | | | | | |
| A | PBS | 0.034 | 0.033 | 0.034 | 0.034 | 0.034 | 0.034 | 0.098 | 0.072 | 0.059 | 0.217 | 0.294 | 0.051 | H35 |
| B | M-PC | 0.211 | 0.504 | 0.306 | 0.37 | 1.322 | 0.034 | 0.119 | 0.07 | 0.061 | 0.231 | 0.277 | 0.048 | H36 |
| C | H29 | 0.073 | 0.049 | 0.043 | 0.117 | 0.183 | 0.042 | 0.127 | 0.075 | 0.058 | 0.252 | 0.308 | 0.053 | H37 |
| D | H30 | 0.072 | 0.047 | 0.053 | 0.378 | 0.144 | 0.048 | 0.084 | 0.053 | 0.053 | 0.079 | 0.196 | 0.046 | H38 |
| E | H31 | 0.12 | 0.066 | 0.053 | 0.137 | 0.242 | 0.05 | 0.157 | 0.082 | 0.064 | 0.347 | 0.269 | 0.049 | H39 |
| F | H32 | 0.509 | 0.067 | 0.054 | 0.416 | 0.458 | 0.045 | 0.157 | 0.068 | 0.05 | 0.21 | 0.257 | 0.047 | H40 |
| G | H33 | 0.167 | 0.061 | 0.054 | 0.392 | 0.28 | 0.044 | 0.192 | 0.071 | 0.058 | 0.381 | 0.358 | 0.043 | H41 |
| H | H34 | 0.151 | 0.06 | 0.047 | 0.165 | 0.293 | 0.041 | 0.127 | 0.059 | 0.054 | 0.19 | 0.353 | 0.051 | H42 |
| 20-04-P4 | | | | | | | | | | | | | | |
| A | PBS | 0.034 | 0.034 | 0.034 | 0.037 | 0.036 | 0.033 | 0.165 | 0.101 | 0.083 | 0.57 | 0.322 | 0.077 | H49 |
| B | M-PC | 0.212 | 0.535 | 0.322 | 0.4 | 1.355 | 0.034 | 0.116 | 0.079 | 0.066 | 0.142 | 0.287 | 0.061 | H50 |
| C | H43 | 0.122 | 0.081 | 0.061 | 0.231 | 0.274 | 0.057 | 0.117 | 0.064 | 0.055 | 0.228 | 0.27 | 0.051 | H51 |
| D | H44 | 0.137 | 0.083 | 0.066 | 0.175 | 0.287 | 0.087 | | | | | | | |
| E | H45 | 0.096 | 0.06 | 0.048 | 0.167 | 0.186 | 0.05 | | | | | | | |
| F | H46 | 0.124 | 0.055 | 0.042 | 0.187 | 0.192 | 0.044 | | | | | | | |
| G | H47 | 0.144 | 0.064 | 0.048 | 0.228 | 0.35 | 0.049 | | | | | | | |
| H | H48 | 0.299 | 0.088 | 0.064 | 0.52 | 0.518 | 0.054 | | | | | | | |

TABLE 23
Data Analysis of Survey Results of Healthy People

| RagB-1 | | RagB-2 | | RagB-3 | | RagB-4 | | Cra4S1 | | GST | |
|---|---|---|---|---|---|---|---|---|---|---|---|
| Specimen | Lowest | Specimen | Lowest | Specimen | Lowest | Specimen | Lowest | Specimen | Lowest | Specimen | Lowest |
| number | OD | number | OD | number | OD | number | OD | number | OD | number | OD |
| H9 | 0.078 | H13 | 0.049 | H9 | 0.047 | H9 | 0.098 | H2 | 0.212 | H9 | 0.044 |
| H13 | 0.085 | H15 | 0.049 | H13 | 0.041 | H13 | 0.116 | H9 | 0.170 | H13 | 0.035 |
| H15 | 0.073 | H18 | 0.054 | H15 | 0.044 | H18 | 0.134 | H13 | 0.124 | H15 | 0.043 |
| H20 | 0.096 | H20 | 0.050 | H18 | 0.044 | H20 | 0.151 | H23 | 0.189 | H18 | 0.044 |
| H23 | 0.071 | H23 | 0.046 | H20 | 0.037 | H23 | 0.071 | H27 | 0.212 | H20 | 0.039 |
| H24 | 0.094 | H24 | 0.056 | H23 | 0.044 | H27 | 0.095 | H29 | 0.183 | H23 | 0.038 |
| H29 | 0.073 | H29 | 0.049 | H27 | 0.043 | H29 | 0.117 | H30 | 0.144 | H27 | 0.038 |
| H30 | 0.072 | H30 | 0.047 | H29 | 0.043 | H31 | 0.137 | H38 | 0.196 | H29 | 0.042 |
| H38 | 0.084 | H38 | 0.053 | H34 | 0.047 | H38 | 0.079 | H45 | 0.186 | H34 | 0.041 |
| H45 | 0.096 | H46 | 0.055 | H46 | 0.042 | H50 | 0.142 | H46 | 0.192 | H41 | 0.043 |
| Average | 0.082 | | 0.051 | | 0.043 | | 0.114 | | 0.181 | | 0.041 |
| 2.5X | 0.206 | | 0.127 | | 0.108 | | 0.285 | | 0.452 | | 0.102 |
| 1.8X | 0.148 | | 0.09 | | 0.078 | | 0.205 | | 0.325 | | 0.073 |
| 4X | 0.329 | | 0.203 | | 0.173 | | 0.456 | | 0.723 | | 0.163 |
| Specimen | | Specimen | | Specimen | | Specimen | | Specimen | | Specimen | |
| number | OD | number | OD | number | OD | number | OD | number | OD | number | OD |

Positive (2.5X)

| | | | | | | | |
|---|---|---|---|---|---|---|---|
| H4 | 0.317 | H7 | 0.195 | H3 | 0.512 | H4 | 0.517 |
| H5 | 0.214 | | | H4 | 0.767 | H25 | 0.46 |
| H6 | 0.211 | | | H6 | 0.32 | H26 | 0.464 |
| H26 | 0.269 | | | H7 | 0.653 | H32 | 0.458 |
| H32 | 0.509 | | | H21 | 0.32 | H48 | 0.518 |
| H48 | 0.299 | | | H22 | 0.415 | | |
| | | | | H25 | 0.501 | | |
| | | | | H26 | 0.824 | | |
| | | | | H28 | 0.376 | | |
| | | | | H30 | 0.378 | | |
| | | | | H32 | 0.416 | | |
| | | | | H33 | 0.392 | | |
| | | | | H39 | 0.347 | | |
| | | | | H41 | 0.381 | | |
| | | | | H48 | 0.52 | | |
| | | | | H49 | 0.57 | | |

Suspicious positive (1.8-2.5X)

| | | | | | | | | | | | |
|---|---|---|---|---|---|---|---|---|---|---|---|
| H12 | 0.181 | H4 | 0.112 | H49 | 0.083 | H1 | 0.25 | H1 | 0.341 | H44 | 0.087 |
| H16 | 0.153 | H7 | 0.099 | | | H5 | 0.233 | H6 | 0.423 | H49 | 0.077 |
| H17 | 0.16 | H26 | 0.1 | | | H8 | 0.258 | H12 | 0.424 | | |
| H19 | 0.175 | H49 | 0.101 | | | H12 | 0.284 | H17 | 0.396 | | |
| H25 | 0.169 | | | | | H14 | 0.232 | H19 | 0.397 | | |
| H28 | 0.147 | | | | | H16 | 0.28 | H28 | 0.346 | | |
| H33 | 0.167 | | | | | H17 | 0.28 | H41 | 0.358 | | |
| H34 | 0.151 | | | | | H19 | 0.234 | H47 | 0.35 | | |
| H39 | 0.157 | | | | | H35 | 0.217 | H49 | 0.322 | | |
| H40 | 0.157 | | | | | H36 | 0.231 | | | | |
| H41 | 0.192 | | | | | H37 | 0.252 | | | | |
| H49 | 0.165 | | | | | H40 | 0.21 | | | | |
| | | | | | | H43 | 0.231 | | | | |
| | | | | | | H47 | 0.228 | | | | |
| | | | | | | H51 | 0.228 | | | | |

TABLE 24
The Distribution of Antibodies to the Outer Membrane Proteins

| | Negative | <1.8X | 1.8-2.5X | 2.5-4X | >4X | Total |
|---|---|---|---|---|---|---|
| Individual | | | | | | |
| Anti-RagB-1 | 10 | 23 | 12 | 5 | 1 | 51 |
| Anti-RagB-2 | 10 | 37 | 4 | 0 | 0 | 51 |
| Anti-RagB-3 | 10 | 39 | 1 | 0 | 1 | 51 |
| Anti-RagB-4 | 10 | 10 | 15 | 9 | 7 | 51 |
| Anti-Cra4S1 | 10 | 27 | 9 | 5 | 0 | 51 |
| Anti-GST | 10 | 39 | 2 | 0 | 0 | 51 |
| Percentage | | | | | | |
| Anti-RagB-1 | 19.6% | 45.1% | 23.5% | 9.8% | 2.0% | 100.0% |
| Anti-RagB-2 | 19.6% | 72.5% | 7.8% | 0.0% | 0.0% | 100.0% |
| Anti-RagB-3 | 19.6% | 76.5% | 2.0% | 0.0% | 2.0% | 100.0% |
| Anti-RagB-4 | 19.6% | 19.6% | 29.4% | 17.6% | 13.7% | 100.0% |
| Anti-Cra4S1 | 19.6% | 52.9% | 17.6% | 9.8% | 0.0% | 100.0% |
| Anti-GST | 19.6% | 76.5% | 3.9% | 0.0% | 0.0% | 100.0% |

TABLE 25
The Distribution of P. gingivalis ragB Alleles in Periodontal Disease

| | Negative | ragB-1 | ragB-2 | ragB-3 | ragB-4 | unknown | Total |
|---|---|---|---|---|---|---|---|
| Individuals | 28 | 7 | 30 | 32 | 6 | 4 | 107 |
| Percentage | 26.2% | 6.5% | 28.0% | 29.9% | 5.6% | 3.7% | 100.0% |

TABLE 26
Investigation of P. gingivalis Antibodies in Sera of Geriatric Patients

| | Specimen | | | | | | | | | | | | | Specimen |
|---|---|---|---|---|---|---|---|---|---|---|---|---|---|---|
| | number | RagB-1 | RagB-2 | RagB-3 | RagB-4 | Cra4S1 | GST | RagB-1 | RagB-2 | RagB-3 | RagB-4 | Cra4S1 | GST | number |
| 20-05-P5 | | | | | | | | | | | | | | |
| A | PBS | 0.037 | 0.036 | 0.035 | 0.036 | 0.036 | 0.036 | 0.178 | 0.146 | 0.138 | 0.151 | 0.154 | 0.084 | 207 |
| B | M-PC | 0.241 | 0.617 | 0.464 | 0.394 | 1.757 | 0.038 | 0.221 | 0.195 | 0.177 | 0.269 | 0.204 | 0.129 | 208 |
| C | 201 | 0.317 | 0.256 | 0.205 | 0.268 | 0.339 | 0.202 | 0.207 | 0.15 | 0.148 | 0.139 | 0.14 | 0.087 | 209 |
| D | 202 | 0.147 | 0.114 | 0.105 | 0.194 | 0.37 | 0.09 | 0.365 | 0.304 | 0.3 | 0.266 | 0.251 | 0.179 | 210 |
| E | 203 | 0.197 | 0.166 | 0.17 | 0.198 | 0.3 | 0.134 | 0.052 | 0.046 | 0.042 | 0.085 | 0.115 | 0.042 | 211 |
| F | 204 | 0.131 | 0.11 | 0.099 | 0.126 | 0.18 | 0.08 | 0.415 | 0.315 | 0.255 | 0.634 | 0.545 | 0.158 | 212 |
| G | 205 | 0.146 | 0.145 | 0.125 | 0.203 | 0.173 | 0.099 | 0.352 | 0.326 | 0.264 | 0.388 | 0.307 | 0.136 | 213 |
| H | 206 | 0.373 | 0.352 | 0.318 | 0.312 | 0.382 | 0.306 | 0.289 | 0.284 | 0.254 | 0.24 | 0.229 | 0.153 | 214 |
| 20-05-P6 | | | | | | | | | | | | | | |
| A | PBS | 0.035 | 0.036 | 0.035 | 0.037 | 0.037 | 0.036 | 0.344 | 0.323 | 0.276 | 0.283 | 0.318 | 0.24 | 221 |
| B | M-PC | 0.25 | 0.608 | 0.523 | 0.386 | 1.708 | 0.038 | 0.083 | 0.069 | 0.063 | 0.2 | 0.087 | 0.058 | 222 |
| C | 215 | 0.31 | 0.237 | 0.221 | 0.306 | 0.272 | 0.209 | 0.156 | 0.097 | 0.092 | 0.209 | 0.172 | 0.077 | 223 |
| D | 216 | 0.303 | 0.231 | 0.223 | 0.253 | 0.245 | 0.213 | 0.376 | 0.316 | 0.279 | 0.346 | 0.323 | 0.239 | 224 |
| E | 217 | 0.187 | 0.157 | 0.133 | 0.152 | 0.163 | 0.123 | 0.163 | 0.13 | 0.115 | 0.193 | 0.193 | 0.099 | 225 |
| F | 218 | 0.062 | 0.05 | 0.049 | 0.201 | 0.077 | 0.039 | 0.318 | 0.179 | 0.17 | 1.056 | 0.183 | 0.121 | 226 |
| G | 219 | 0.218 | 0.171 | 0.162 | 0.26 | 0.442 | 0.122 | 0.105 | 0.072 | 0.063 | 0.13 | 0.164 | 0.07 | 227 |
| H | 220 | 0.294 | 0.207 | 0.201 | 0.235 | 0.268 | 0.187 | 0.056 | 0.045 | 0.046 | 0.056 | 0.086 | 0.053 | 228 |
| 20-05-P7 | | | | | | | | | | | | | | |
| A | PBS | 0.037 | 0.038 | 0.036 | 0.038 | 0.038 | 0.036 | 0.422 | 0.366 | 0.304 | 0.322 | 0.323 | 0.286 | 235 |
| B | M-PC | 0.28 | 0.667 | 0.546 | 0.449 | 1.859 | 0.039 | 0.691 | 0.64 | 0.516 | 0.704 | 0.65 | 0.416 | 236 |
| C | 229 | 0.112 | 0.083 | 0.076 | 0.15 | 0.159 | 0.068 | 0.219 | 0.176 | 0.171 | 0.215 | 0.182 | 0.134 | 237 |
| D | 230 | 0.092 | 0.074 | 0.063 | 0.084 | 0.117 | 0.057 | 0.213 | 0.182 | 0.151 | 0.178 | 0.183 | 0.125 | 238 |
| E | 231 | 0.104 | 0.102 | 0.086 | 0.095 | 0.101 | 0.083 | 0.107 | 0.089 | 0.08 | 0.09 | 0.091 | 0.069 | 239 |
| F | 232 | 0.302 | 0.248 | 0.23 | 0.433 | 0.219 | 0.153 | 0.674 | 0.562 | 0.491 | 0.681 | 0.549 | 0.354 | 240 |
| G | 233 | 0.308 | 0.255 | 0.235 | 0.229 | 0.234 | 0.172 | 0.224 | 0.165 | 0.139 | 0.287 | 0.29 | 0.116 | 241 |
| H | 234 | 0.317 | 0.267 | 0.236 | 0.371 | 0.314 | 0.185 | 0.204 | 0.159 | 0.148 | 0.28 | 0.203 | 0.121 | 242 |
| 20-05-P8 | | | | | | | | | | | | | | |
| A | PBS | 0.04 | 0.041 | 0.043 | 0.039 | 0.038 | 0.036 | 0.715 | 0.569 | 0.496 | 0.725 | 0.963 | 0.425 | 249 |
| B | M-PC | 0.307 | 0.648 | 0.554 | 0.419 | 1.735 | 0.039 | 0.713 | 0.613 | 0.526 | 0.795 | 0.671 | 0.467 | 250 |
| C | 243 | 0.834 | 1.33 | 0.711 | 0.787 | 0.608 | 0.597 | 0.164 | 0.141 | 0.124 | 0.212 | 0.228 | 0.1 | 251 |
| D | 244 | 0.535 | 0.398 | 0.381 | 0.782 | 0.705 | 0.322 | 0.848 | 0.745 | 0.635 | 0.73 | 0.625 | 0.52 | 252 |
| E | 245 | 0.802 | 0.452 | 0.39 | 1.598 | 0.537 | 0.292 | 0.751 | 0.638 | 0.566 | 1.013 | 1.006 | 0.479 | 253 |
| F | 246 | 0.583 | 0.417 | 0.354 | 0.781 | 0.499 | 0.268 | 0.176 | 0.164 | 0.148 | 0.191 | 0.178 | 0.118 | 254 |
| G | 247 | 0.307 | 0.199 | 0.162 | 1.0 | 0.387 | 0.106 | 0.274 | 0.162 | 0.119 | 0.875 | 0.339 | 0.102 | 255 |
| H | 248 | 0.429 | 0.3 | 0.29 | 0.452 | 0.546 | 0.25 | | | | | | | |

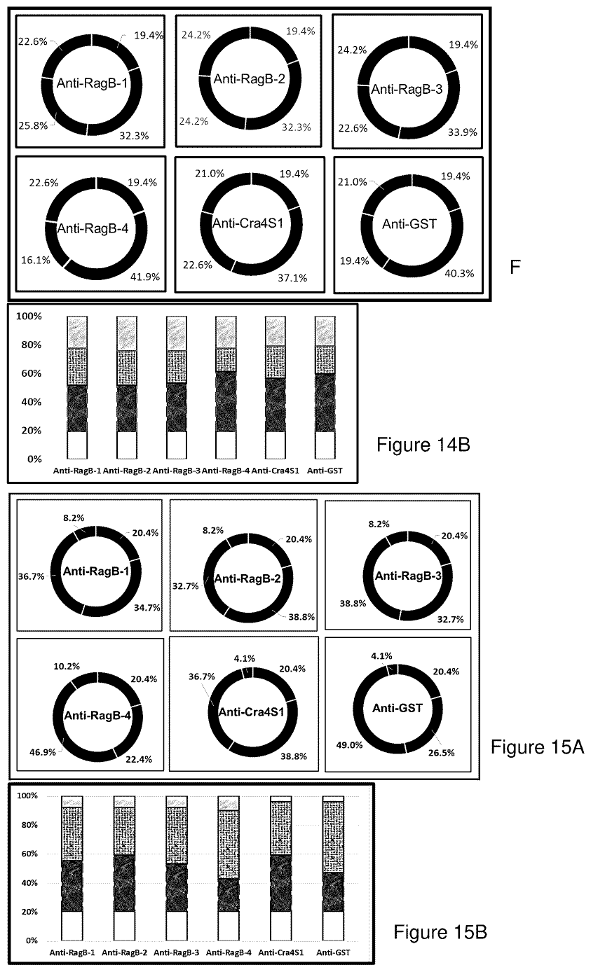

Table 27 shows the data analysis of the survey of serum samples from geriatric patients (sample 201-255 in Table 26; sample 256-262 in Table 29), total 62 samples. According to the preset standard, Table 28 shows the data analysis of the survey of serum samples from geriatric patients, the positive rate of RagB-1 antibody detection was 48.4% (30/62), of which the strong positive rate was 22.6% (14/62); the positive rate of RagB-2 was 48.4% (30/62), of which the strong positive rate was 24.2% (15/62); the positive rate of RagB-3 antibody was 46.8% (29/62), of which the strong positive rate was 24.2% (15/62); the positive rate of RagB-4 antibody was 38.7% (24/62), of which the strong positive rate was 22.6% (14/62); the positive rate of Cra4S1 antibody was 43.5% (27/62), of which the strong positive rate was 21% (13/62); and the positive rate of unrelated protein GST antibody was 40.3% (25/62), of which the strong positive rate was 21% (13/62), see attached FIGS. 14A-14B.

TABLE 27
Data Analysis of Survey Results from Geriatric Patients

| | RagB-1 | | RagB-2 | | RagB-3 | | RagB-4 | | Cra4S1 | | GST | |
|---|---|---|---|---|---|---|---|---|---|---|---|---|
| Number | Specimen | Lowest | Specimen | Lowest | Specimen | Lowest | Specimen | Lowest | Specimen | Lowest | Specimen | Lowest |
| sequence | number | OD | number | OD | number | OD | number | OD | number | OD | number | OD |
| 1 | 202 | 0.147 | 202 | 0.114 | 202 | 0.105 | 204 | 0.126 | 207 | 0.154 | 204 | 0.08 |
| 2 | 204 | 0.131 | 204 | 0.11 | 204 | 0.099 | 207 | 0.151 | 209 | 0.14 | 207 | 0.084 |
| 3 | 205 | 0.146 | 211 | 0.046 | 211 | 0.042 | 209 | 0.139 | 211 | 0.115 | 211 | 0.042 |
| 4 | 211 | 0.052 | 218 | 0.05 | 218 | 0.049 | 211 | 0.085 | 217 | 0.163 | 218 | 0.039 |
| 5 | 218 | 0.062 | 222 | 0.069 | 222 | 0.063 | 217 | 0.152 | 218 | 0.077 | 222 | 0.058 |
| 6 | 222 | 0.083 | 223 | 0.097 | 223 | 0.092 | 227 | 0.13 | 222 | 0.087 | 223 | 0.077 |
| 7 | 227 | 0.105 | 227 | 0.072 | 227 | 0.063 | 228 | 0.056 | 227 | 0.164 | 227 | 0.07 |
| 8 | 228 | 0.056 | 228 | 0.045 | 228 | 0.046 | 229 | 0.15 | 228 | 0.086 | 228 | 0.053 |
| 9 | 229 | 0.112 | 229 | 0.083 | 229 | 0.076 | 230 | 0.084 | 229 | 0.159 | 229 | 0.068 |
| 10 | 230 | 0.092 | 230 | 0.074 | 230 | 0.063 | 231 | 0.095 | 230 | 0.117 | 230 | 0.057 |
| 11 | 231 | 0.104 | 231 | 0.102 | 231 | 0.086 | 238 | 0.187 | 231 | 0.101 | 231 | 0.083 |
| 12 | 239 | 0.107 | 239 | 0.089 | 239 | 0.08 | 239 | 0.09 | 239 | 0.091 | 239 | 0.069 |
| | Average | 0.095 | | 0.079 | | 0.069 | | 0.120 | | 0.121 | | 0.065 |
| | 2.5X | 0.239 | | 0.198 | | 0.173 | | 0.301 | | 0.303 | | 0.163 |
| | 4X | 0.38 | | 0.32 | | 0.28 | | 0.48 | | 0.48 | | 0.26 |
| | Specimen | | Specimen | | Specimen | | Specimen | | Specimen | | Specimen | |
| | number | OD | number | OD | number | OD | number | OD | number | OD | number | OD |

Positive antibody (2.5X)

| | | | | | | | | | | | | |
|---|---|---|---|---|---|---|---|---|---|---|---|---|
| 1 | 201 | 0.317 | 201 | 0.256 | 201 | 0.205 | 206 | 0.312 | 201 | 0.339 | 201 | 0.202 |
| 2 | 206 | 0.373 | 208 | 0.195 | 208 | 0.177 | 213 | 0.388 | 202 | 0.37 | 210 | 0.179 |
| 3 | 210 | 0.365 | 210 | 0.304 | 212 | 0.255 | 215 | 0.306 | 203 | 0.3 | 212 | 0.158 |
| 4 | 213 | 0.352 | 212 | 0.315 | 213 | 0.264 | 224 | 0.346 | 206 | 0.382 | 215 | 0.209 |
| 5 | 214 | 0.289 | 214 | 0.284 | 214 | 0.254 | 232 | 0.433 | 213 | 0.307 | 216 | 0.213 |
| 6 | 215 | 0.31 | 215 | 0.237 | 215 | 0.221 | 234 | 0.371 | 219 | 0.442 | 220 | 0.187 |
| 7 | 216 | 0.303 | 216 | 0.231 | 216 | 0.223 | 235 | 0.322 | 221 | 0.318 | 221 | 0.24 |
| 8 | 220 | 0.294 | 220 | 0.207 | 220 | 0.201 | 241 | 0.287 | 224 | 0.323 | 224 | 0.239 |
| 9 | 221 | 0.344 | 224 | 0.316 | 221 | 0.276 | 248 | 0.452 | 234 | 0.314 | 233 | 0.172 |
| 10 | 224 | 0.376 | 232 | 0.248 | 224 | 0.279 | | | 235 | 0.323 | 234 | 0.185 |
| 11 | 226 | 0.318 | 233 | 0.255 | 232 | 0.23 | | | 241 | 0.29 | 248 | 0.25 |
| 12 | 232 | 0.302 | 234 | 0.267 | 233 | 0.235 | | | 247 | 0.387 | | |
| 13 | 233 | 0.308 | 247 | 0.199 | 234 | 0.236 | | | 255 | 0.339 | | |
| 14 | 234 | 0.317 | 248 | 0.3 | | | | | | | | |
| 15 | 247 | 0.307 | | | | | | | | | | |
| 16 | 255 | 0.274 | | | | | | | | | | |

Positive antibody (4X)

| | | | | | | | | | | | | |
|---|---|---|---|---|---|---|---|---|---|---|---|---|
| 1 | 212 | 0.415 | 206 | 0.352 | 206 | 0.318 | 212 | 0.634 | 212 | 0.545 | 206 | 0.306 |
| 2 | 235 | 0.422 | 213 | 0.326 | 210 | 0.3 | 226 | 1.056 | 236 | 0.65 | 235 | 0.286 |
| 3 | 236 | 0.691 | 221 | 0.323 | 235 | 0.304 | 236 | 0.704 | 240 | 0.549 | 236 | 0.416 |
| 4 | 240 | 0.674 | 235 | 0.366 | 236 | 0.516 | 240 | 0.681 | 243 | 0.608 | 240 | 0.354 |
| 5 | 243 | 0.834 | 236 | 0.64 | 240 | 0.491 | 243 | 0.787 | 244 | 0.705 | 243 | 0.597 |
| 6 | 244 | 0.535 | 240 | 0.562 | 243 | 0.711 | 244 | 0.782 | 245 | 0.537 | 244 | 0.322 |
| 7 | 245 | 0.802 | 243 | 1.33 | 244 | 0.381 | 245 | 1.598 | 246 | 0.499 | 245 | 0.292 |
| 8 | 246 | 0.583 | 244 | 0.398 | 245 | 0.39 | 246 | 0.781 | 248 | 0.546 | 246 | 0.268 |
| 9 | 248 | 0.429 | 245 | 0.452 | 246 | 0.354 | 247 | 1.0 | 249 | 0.963 | 249 | 0.425 |
| 10 | 249 | 0.715 | 246 | 0.417 | 248 | 0.29 | 249 | 0.725 | 250 | 0.671 | 250 | 0.467 |
| 11 | 250 | 0.713 | 249 | 0.569 | 249 | 0.496 | 250 | 0.795 | 252 | 0.625 | 252 | 0.52 |
| 12 | 252 | 0.848 | 250 | 0.613 | 250 | 0.526 | 252 | 0.73 | 253 | 1.006 | 253 | 0.479 |
| 13 | 253 | 0.751 | 252 | 0.745 | 252 | 0.635 | 253 | 1.013 | | | | |
| 14 | | | 253 | 0.638 | 253 | 0.566 | 255 | 0.875 | | | | |

TABLE 28
The Distribution of Antibodies to the Outer Membrane Proteins

| | Negative | <2.5X | 2.5-4X | >4X | Total specimen |
|---|---|---|---|---|---|
| Individual | | | | | |
| Anti-RagB-1 | 12 | 20 | 16 | 14 | 62 |
| Anti-RagB-2 | 12 | 20 | 15 | 15 | 62 |
| Anti-RagB-3 | 12 | 21 | 14 | 15 | 62 |
| Anti-RagB-4 | 12 | 26 | 10 | 14 | 62 |
| Anti-Cra4S1 | 12 | 23 | 14 | 13 | 62 |
| Anti-GST | 12 | 25 | 12 | 13 | 62 |
| Percentage | | | | | |
| Anti-RagB-1 | 19.4% | 32.3% | 25.8% | 22.6% | 100.0% |
| Anti-RagB-2 | 19.4% | 32.3% | 24.2% | 24.2% | 100.0% |
| Anti-RagB-3 | 19.4% | 33.9% | 22.6% | 24.2% | 100.0% |
| Anti-RagB-4 | 19.4% | 41.9% | 16.1% | 22.6% | 100.0% |
| Anti-Cra4S1 | 19.4% | 37.1% | 22.6% | 21.0% | 100.0% |
| Anti-GST | 19.4% | 40.3% | 19.4% | 21.0% | 100.0% |

TABLE 29
Investigation of P. gingivalis Antibodies in Sera of Cardiovascular Patients

| | Specimen | | | | | | | | | | | | | Specimen |
|---|---|---|---|---|---|---|---|---|---|---|---|---|---|---|
| | number | RagB-1 | RagB-2 | RagB-3 | RagB-4 | Cra4S1 | GST | RagB-1 | RagB-2 | RagB-3 | RagB-4 | Cra4S1 | GST | number |
| 20-05-P9 | | | | | | | | | | | | | | |
| A | PBS | 0.033 | 0.034 | 0.037 | 0.034 | 0.036 | 0.035 | 0.407 | 0.419 | 0.364 | 0.414 | 0.425 | 0.325 | 262 |
| B | M-PC | 0.247 | 0.568 | 0.51 | 0.374 | 1.647 | 0.037 | 0.108 | 0.108 | 0.106 | 0.111 | 0.121 | 0.091 | 101 |
| C | 256 | 0.168 | 0.165 | 0.152 | 0.18 | 0.203 | 0.159 | 0.1 | 0.097 | 0.105 | 0.106 | 0.116 | 0.086 | 102 |
| D | 257 | 0.045 | 0.05 | 0.051 | 0.058 | 0.066 | 0.052 | 0.239 | 0.23 | 0.225 | 0.265 | 0.271 | 0.207 | 103 |
| E | 258 | 0.078 | 0.083 | 0.082 | 0.088 | 0.096 | 0.077 | 0.299 | 0.297 | 0.273 | 0.295 | 0.3 | 0.248 | 104 |
| F | 259 | 0.133 | 0.128 | 0.114 | 0.151 | 0.155 | 0.108 | 0.139 | 0.129 | 0.121 | 0.149 | 0.115 | 0.096 | 105 |
| G | 260 | 0.074 | 0.081 | 0.077 | 0.09 | 0.082 | 0.074 | 0.106 | 0.097 | 0.098 | 0.107 | 0.114 | 0.087 | 106 |
| H | 261 | 0.005 | 0.009 | 0.056 | 0.032 | 0.039 | 0.023 | 0.072 | 0.066 | 0.077 | 0.056 | 0.13 | 0.035 | 107 |
| 20-05-P10 | | | | | | | | | | | | | | |
| A | PBS | 0.036 | 0.036 | 0.034 | 0.035 | 0.036 | 0.036 | 0.057 | 0.054 | 0.049 | 0.051 | 0.051 | 0.045 | 114 |
| B | M-PC | 0.295 | 0.567 | 0.567 | 0.38 | 1.63 | 0.037 | 0.232 | 0.208 | 0.189 | 0.209 | 0.234 | 0.177 | 115 |
| C | 108 | 0.219 | 0.179 | 0.187 | 0.193 | 0.231 | 0.186 | 0.258 | 0.223 | 0.222 | 0.233 | 0.25 | 0.201 | 116 |
| D | 109 | 0.149 | 0.145 | 0.129 | 0.148 | 0.151 | 0.126 | 0.235 | 0.222 | 0.201 | 0.23 | 0.241 | 0.19 | 117 |
| E | 110 | 0.156 | 0.152 | 0.129 | 0.172 | 0.146 | 0.14 | 0.274 | 0.244 | 0.216 | 0.244 | 0.26 | 0.206 | 118 |
| F | 111 | 0.069 | 0.058 | 0.052 | 0.06 | 0.069 | 0.053 | 0.185 | 0.157 | 0.149 | 0.156 | 0.167 | 0.138 | 119 |
| G | 112 | 0.322 | 0.267 | 0.258 | 0.292 | 0.324 | 0.239 | 0.141 | 0.122 | 0.106 | 0.108 | 0.104 | 0.097 | 120 |
| H | 113 | 0.144 | 0.135 | 0.137 | 0.158 | 0.145 | 0.125 | 0.089 | 0.086 | 0.075 | 0.082 | 0.095 | 0.071 | 121 |
| 20-05-P11 | | | | | | | | | | | | | | |
| A | PBS | 0.026 | 0.026 | 0.027 | 0.027 | 0.027 | 0.031 | 0.05 | 0.045 | 0.041 | 0.054 | 0.065 | 0.038 | 128 |
| B | M-PC | 0.294 | 0.579 | 0.513 | 0.411 | 1.477 | 0.032 | 0.062 | 0.056 | 0.051 | 0.064 | 0.069 | 0.053 | 129 |
| C | 122 | 0.083 | 0.07 | 0.068 | 0.08 | 0.103 | 0.07 | 0.202 | 0.181 | 0.168 | 0.17 | 0.157 | 0.137 | 130 |
| D | 123 | 0.235 | 0.216 | 0.198 | 0.211 | 0.21 | 0.177 | 0.119 | 0.109 | 0.103 | 0.12 | 0.12 | 0.095 | 131 |
| E | 124 | 0.162 | 0.147 | 0.135 | 0.175 | 0.144 | 0.12 | 0.192 | 0.174 | 0.161 | 0.185 | 0.187 | 0.155 | 132 |
| F | 125 | 0.168 | 0.152 | 0.145 | 0.167 | 0.165 | 0.14 | 0.19 | 0.178 | 0.159 | 0.187 | 0.172 | 0.149 | 133 |
| G | 126 | 0.116 | 0.103 | 0.091 | 0.097 | 0.106 | 0.086 | 0.15 | 0.139 | 0.135 | 0.169 | 0.135 | 0.121 | 134 |
| H | 127 | 0.274 | 0.229 | 0.186 | 0.222 | 0.239 | 0.189 | 0.127 | 0.11 | 0.116 | 0.135 | 0.127 | 0.107 | 135 |
| 20-05-P12 | | | | | | | | | | | | | | |
| A | PBS | 0.035 | 0.035 | 0.035 | 0.036 | 0.037 | 0.035 | 0.287 | 0.244 | 0.227 | 0.231 | 0.257 | 0.174 | 142 |
| B | M-PC | 0.304 | 0.664 | 0.531 | 0.469 | 1.715 | 0.037 | 0.122 | 0.097 | 0.093 | 0.109 | 0.131 | 0.081 | 143 |
| C | 136 | 0.2 | 0.168 | 0.153 | 0.177 | 0.214 | 0.139 | 0.232 | 0.191 | 0.19 | 0.175 | 0.17 | 0.14 | 144 |
| D | 137 | 0.08 | 0.073 | 0.068 | 0.075 | 0.073 | 0.06 | 0.046 | 0.045 | 0.042 | 0.045 | 0.06 | 0.042 | 145 |
| E | 138 | 0.138 | 0.131 | 0.127 | 0.174 | 0.136 | 0.113 | 0.091 | 0.078 | 0.069 | 0.073 | 0.083 | 0.063 | 146 |
| F | 139 | 0.075 | 0.068 | 0.061 | 0.082 | 0.086 | 0.058 | 0.219 | 0.176 | 0.16 | 0.182 | 0.207 | 0.136 | 147 |
| G | 140 | 0.319 | 0.269 | 0.254 | 0.275 | 0.274 | 0.207 | 0.227 | 0.203 | 0.191 | 0.185 | 0.218 | 0.17 | 148 |
| H | 141 | 0.264 | 0.264 | 0.244 | 0.261 | 0.25 | 0.194 | 0.185 | 0.16 | 0.159 | 0.16 | 0.189 | 0.142 | 149 |

TABLE 30
Data Analysis of Survey Results of Cardiovascular Patients

| | RagB-1 | | RagB-2 | | RagB-3 | | RagB-4 | | Cra4S1 | | GST | |
|---|---|---|---|---|---|---|---|---|---|---|---|---|
| Number | Specimen | Lowest | Specimen | Lowest | Specimen | Lowest | Specimen | Lowest | Specimen | Lowest | Specimen | Lowest |
| sequence | number | OD | number | OD | number | OD | number | OD | number | OD | number | OD |
| 1 | 102 | 0.1 | 106 | 0.098 | 111 | 0.052 | 107 | 0.056 | 111 | 0.069 | 106 | 0.087 |
| 2 | 107 | 0.072 | 107 | 0.066 | 114 | 0.049 | 111 | 0.06 | 114 | 0.051 | 107 | 0.035 |
| 3 | 111 | 0.069 | 111 | 0.058 | 121 | 0.075 | 114 | 0.051 | 121 | 0.095 | 111 | 0.053 |
| 4 | 114 | 0.057 | 114 | 0.054 | 122 | 0.068 | 122 | 0.08 | 126 | 0.097 | 114 | 0.045 |
| 5 | 121 | 0.089 | 122 | 0.07 | 128 | 0.041 | 128 | 0.054 | 128 | 0.065 | 122 | 0.07 |
| 6 | 122 | 0.083 | 128 | 0.045 | 129 | 0.051 | 129 | 0.064 | 129 | 0.069 | 128 | 0.038 |
| 7 | 128 | 0.05 | 129 | 0.056 | 137 | 0.068 | 137 | 0.075 | 137 | 0.073 | 129 | 0.053 |
| 8 | 129 | 0.062 | 137 | 0.073 | 139 | 0.061 | 139 | 0.082 | 139 | 0.086 | 137 | 0.06 |
| 9 | 139 | 0.075 | 139 | 0.068 | 145 | 0.042 | 145 | 0.045 | 145 | 0.06 | 139 | 0.058 |
| 10 | 145 | 0.046 | 145 | 0.045 | 146 | 0.069 | 146 | 0.073 | 146 | 0.083 | 145 | 0.042 |
| | Average | 0.070 | Average | 0.0633 | Average | 0.0576 | Average | 0.064 | Average | 0.0748 | Average | 0.0541 |
| | 2.5X | 0.176 | 2.5X | 0.158 | 2.5X | 0.144 | 2.5X | 0.160 | 2.5X | 0.187 | 2.5X | 0.135 |
| | 4X | 0.2812 | 4X | 0.253 | 4X | 0.230 | 4X | 0.256 | 4X | 0.299 | 4X | 0.216 |
| | Specimen | | Specimen | | Specimen | | Specimen | | Specimen | | Specimen | |
| | number | OD | number | OD | number | OD | number | OD | number | OD | number | OD |

Positive antibody (2.5X)

| | | | | | | | | | | | | |
|---|---|---|---|---|---|---|---|---|---|---|---|---|
| 1 | 103 | 0.239 | 103 | 0.23 | 103 | 0.225 | 105 | 0.149 | 103 | 0.271 | 103 | 0.207 |
| 2 | 108 | 0.219 | 108 | 0.179 | 108 | 0.187 | 108 | 0.193 | 108 | 0.231 | 104 | 0.248 |
| 3 | 115 | 0.232 | 115 | 0.208 | 115 | 0.189 | 110 | 0.172 | 115 | 0.234 | 108 | 0.186 |
| 4 | 116 | 0.258 | 116 | 0.223 | 116 | 0.222 | 113 | 0.158 | 116 | 0.25 | 110 | 0.14 |
| 5 | 117 | 0.235 | 117 | 0.222 | 117 | 0.201 | 115 | 0.209 | 117 | 0.241 | 115 | 0.177 |
| 6 | 118 | 0.274 | 118 | 0.244 | 118 | 0.216 | 116 | 0.233 | 118 | 0.26 | 116 | 0.201 |
| 7 | 119 | 0.185 | 127 | 0.229 | 119 | 0.149 | 117 | 0.23 | 123 | 0.21 | 117 | 0.19 |
| 8 | 123 | 0.235 | 130 | 0.181 | 123 | 0.198 | 118 | 0.244 | 127 | 0.239 | 118 | 0.206 |
| 9 | 127 | 0.274 | 132 | 0.174 | 125 | 0.145 | 123 | 0.211 | 132 | 0.187 | 119 | 0.138 |
| 10 | 130 | 0.202 | 133 | 0.178 | 127 | 0.186 | 124 | 0.175 | 133 | 0.172 | 123 | 0.177 |
| 11 | 132 | 0.192 | 136 | 0.168 | 130 | 0.168 | 125 | 0.167 | 136 | 0.214 | 125 | 0.14 |
| 12 | 133 | 0.19 | 142 | 0.244 | 132 | 0.161 | 127 | 0.222 | 140 | 0.274 | 127 | 0.189 |
| 13 | 136 | 0.2 | 144 | 0.191 | 133 | 0.159 | 130 | 0.17 | 141 | 0.25 | 130 | 0.137 |
| 14 | 141 | 0.264 | 147 | 0.176 | 136 | 0.153 | 132 | 0.185 | 142 | 0.257 | 132 | 0.155 |
| 15 | 144 | 0.232 | 148 | 0.203 | 142 | 0.227 | 133 | 0.187 | 144 | 0.17 | 133 | 0.149 |
| 16 | 147 | 0.219 | 149 | 0.16 | 144 | 0.19 | 134 | 0.169 | 147 | 0.207 | 134 | 0.121 |
| 17 | 148 | 0.227 | | | 147 | 0.16 | 136 | 0.177 | 148 | 0.218 | 136 | 0.139 |
| 18 | 149 | 0.185 | | | 148 | 0.191 | 138 | 0.174 | 149 | 0.189 | 140 | 0.207 |
| 19 | | | | | 149 | 0.159 | 142 | 0.231 | | | 141 | 0.194 |
| 20 | | | | | | | 144 | 0.175 | | | 142 | 0.174 |
| 21 | | | | | | | 147 | 0.182 | | | 144 | 0.14 |
| 22 | | | | | | | 148 | 0.185 | | | 147 | 0.136 |
| 23 | | | | | | | 149 | 0.16 | | | 148 | 0.17 |
| 24 | | | | | | | | | | | 149 | 0.142 |

Positive antibody (4X)

| | | | | | | | | | | | | |
|---|---|---|---|---|---|---|---|---|---|---|---|---|
| 1 | 104 | 0.299 | 104 | 0.297 | 104 | 0.273 | 103 | 0.265 | 104 | 0.3 | 104 | 0.248 |
| 2 | 112 | 0.322 | 112 | 0.267 | 112 | 0.258 | 104 | 0.295 | 112 | 0.324 | 112 | 0.239 |
| 3 | 140 | 0.319 | 140 | 0.269 | 140 | 0.254 | 112 | 0.292 | | | | |
| 4 | 142 | 0.287 | 141 | 0.264 | 141 | 0.244 | 140 | 0.275 | | | | |
| 5 | | | | | | | 141 | 0.261 | | | | |

Table 29 shows the original ELISA results of serological detection in cardiovascular patients (and some geriatric patients, see details above). Briefly, antigen coating and assay procedure were the same as above description, the patient serum in the reaction plate was diluted 1:500.

Table 30 shows the data analysis of the survey of serum samples from cardiovascular patients (No. 101-149), a total of 49 samples. According to the preset standard, the positive rate of RagB-1 antibody detection was 44.9% (22/49), of which the strong positive rate was 8.2% (4/49); the positive rate of RagB-2 was 40.8% (20/49), of which the strong positive rate was 8.2% (4/49); the positive rate of RagB-3 antibody was 46.9% (23/49), of which the strong positive rate was 8.2% (4/49); the positive rate of RagB-4 antibody was 57.1% (28/49), of which the strong positive rate was 10.2% (5/49); and the positive rate of Cra4S1 antibody was 40.8% (20/49), of which the strong positive rate was 4.1% (2/49); and the positive rate of unrelated protein GST antibody was 53.1% (26/49), of which the strong positive rate was 4.1% (2/49); see FIGS. 15A-15B and Table 31.

Serological findings in cardiovascular and geriatric patients had three striking features compared with healthy individuals. First, the detection rate of RagB-2 antibody and RagB-3 antibody was significantly increased; second, the detection rate of GST protein antibody (that is, to an irrelevant antigen) was greatly increased; third, the proportion of patients with high antibody titers in cardiovascular and geriatric patient samples were much higher than in healthy people.

TABLE 31
The Distribution of Antibodies to the Outer Membrane Proteins

| | | | | |
|---|---|---|---|---|
| | | | | Total |
| Negative | <2.5X | 2.5-4X | >4X | specimen |

Individual

| | | | | | |
|---|---|---|---|---|---|
| Anti-RagB-1 | 10 | 17 | 18 | 4 | 49 |
| Anti-RagB-2 | 10 | 19 | 16 | 4 | 49 |
| Anti-RagB-3 | 10 | 16 | 19 | 4 | 49 |
| Anti-RagB-4 | 10 | 11 | 23 | 5 | 49 |
| Anti-Cra4S1 | 10 | 19 | 18 | 2 | 49 |
| Anti-GST | 10 | 13 | 24 | 2 | 49 |

Percentage

| | | | | | |
|---|---|---|---|---|---|
| Anti-RagB-1 | 20.4% | 34.7% | 36.7% | 8.2% | 100.0% |
| Anti-RagB-2 | 20.4% | 38.8% | 32.7% | 8.2% | 100.0% |
| Anti-RagB-3 | 20.4% | 32.7% | 38.8% | 8.2% | 100.0% |
| Anti-RagB-4 | 20.4% | 22.4% | 46.9% | 10.2% | 100.0% |
| Anti-Cra4S1 | 20.4% | 38.8% | 36.7% | 4.1% | 100.0% |
| Anti-GST | 20.4% | 26.5% | 49.0% | 4.1% | 100.0% |

“Antibody” against the unrelated antigen GST was not detected in healthy subjects, while the detection rate of antibodies to GST in cardiovascular disease patients and geriatric patients was notably present. In addition, the detection of GST antibodies was directly related to the level of RagB and Cra4S1 antibodies and only occurred in patients with these antibodies. The individuals with higher OD values in antibodies to RagB and Cra4S1 also had higher OD values of “GST antibody”. No independent “GST antibody” positive case was found.

The serological antibody study against P. gingivalis outer membrane protein found that a large proportion of patients with cardiovascular disease and geriatric medicine had antibodies to P. gingivalis outer membrane proteins. Importantly, patients with high antibody titers to P. gingivalis outer membrane proteins, especially to RagB-2 and RagB-3 and Cra4S1, also had an increased incidence of antibodies (and high antibody titers) to the unrelated protein GST. These data provide a scientific basis, suggesting an aspect of the pathogenic mechanism of P. gingivalis through heterophilic antigenic properties.

Multiple lines of evidence suggest that P. gingivalis outer membrane proteins, including RagB and Cra4S1, have heterophilic antigenic features. Heterophilic antigens are a class of common antigens that are completely unrelated to, but similar in structure to antigens present in host tissues. Many scientific literature reports describe diseases caused by heterophilic antigens. For example, the cell membrane of hemolytic streptococcus has common antigens with glomerular basement membrane and myocardial tissue, so after streptococcus infection, glomerulonephritis or myocarditis may occur. Similarly, Escherichia coli (E. coli) lipopolysaccharide and human colonic mucosa have common antigens, infection with the former is linked to development of subsequent ulcerative colitis.

Heterophilic antibodies are multi-specific immunoglobulins with a certain titer, induced by known or unknown antigenic substances (heterophilic antigens), and can bind to various proteins, but the affinity may be not very strong. These antibodies can be endogenous and naturally occurring, and they can react with molecules unrelated to the original antigen. These immunoglobulins with different chemical structures but with multiple binding abilities to the tested substance are called heterophilic antibodies.

Heterophilic antibodies can be understood as multi-specific or nonspecific immune enhancement, which may have an immune protective function. However, multiple immune responses of heterophilic antibodies to heterophilic antigens and antigenic components shared in different tissues can cause immunopathology.

Serological findings showed that RagB, Cra4S1, and GST antibody-positive cases in cardiovascular patients and geriatric patients accounted for a large proportion of the overall sample. Combined with epidemiological survey data, P. gingivalis outer membrane proteins, including RagB and Cra4S1, showed the characteristics of heterophilic antigens.

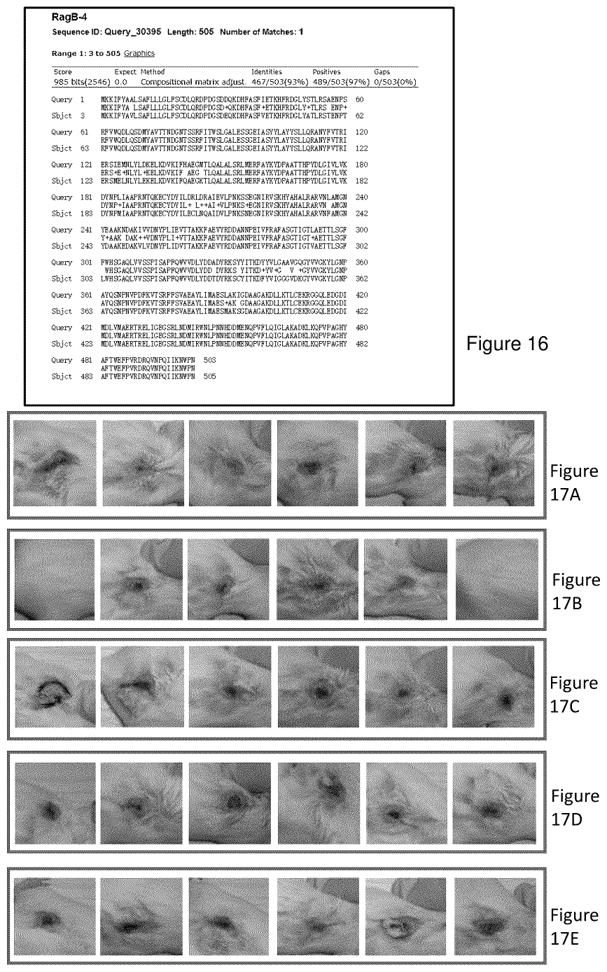

Since the RagB-4 (381 RagB) DNA/protein sequence (SEQ ID NO: 8) was firstly disclosed in 2004, scientists have discovered high similarity rate of protein sequence as RagB-4 in other species, for example, Porphyromonas gulae, a major periodontal pathogen in dogs and cats, it's outer membrane protein (access number WP_039431148) has 93% similarity rate with RagB-4 (FIG. 16), further supporting P. gingivalis heterophilic antigenic characteristics and broad application value. In addition, RagB-4 is not related to the Cra4S1 molecular sequence, but as described in Example 4 and later in Example 7, the RagB-4-1B11-4-4 monoclonal antibody cross reacted with the Cra4S1 protein, this cross-reactivity to the whole Cra4S1 protein rather than a specific proportion of peptide suggests that there may be diffuse or weak binding, a feature which is often observed in heterophilic antigens.

Periodontal disease is a very common disease in humans. P. gingivalis is the main pathogen, and the detection of antibodies against P. gingivalis outer membrane protein in human serum means that the subject has natural antibodies or has a history of P. gingivalis infections. Periodontal disease is usually chronic and progressive, the body having varying degrees of immune response to P. gingivalis but often never fully eradicating the infection.

This Example shows that the detection rate of P. gingivalis outer membrane protein antibody in the serum of cardiovascular disease and geriatric disease patients is relatively high. Additionally, a large proportion of these patients also display reactions between immunoglobulins and irrelevant proteins in the serum. This suggests that a considerable percentage of these patients are in a state of immune sensitization, and these antibodies, likely heterophilic antibodies, are strongly correlated to these chronic diseases and pathological damage, perhaps even playing a causative role in some disease etiology.

As suggested, P. gingivalis appears to be less toxic to young people. It can be postulated that the heterophilic antibodies have not yet accumulated to a harmful level in a younger population. Human serological samples also showed some individuals with no detectable antibodies to any P. gingivalis outer membrane proteins among all three groups studied, including healthy people, cardiovascular patients and geriatric patients (Tables 22, 26 and 29), these individuals either have natural/non-specific resistance to P. gingivalis or have never been infected by P. gingivalis. This finding underlines that P. gingivalis is likely one of many multifactorial contributors of complex system disease, and that the role of individual differences in immune response is important in determining whether an infection will be cleared or persist.

Detection of antibodies against RagB and Cra4S1 antigens as well as GST or other tissue antigens in serum is a simple and easy way to predict the development of P. gingivalis infection-related diseases. This could prompt timely attention to disease development and offer a huge benefit of initiating early treatment of patients, potentially reducing morbidity and mortality.

Further investigations to deepen the understanding of the relationship between P. gingivalis infection and chronic diseases may be needed. However, the data presented in this Example suggest the heterophilic antigenic characteristics of P. gingivalis, this, coupled with the characteristics of individuals, can explain the heterophilic antibodies and/or hypersensitivity reactions which are important factors in chronic P. gingivalis infection and systemic disease. Therefore, reduce tissue damage caused by harmful heterophilic antibodies, actively treat related diseases, and control and prevent the further deterioration in a multitude of tissues can be achieved by eliminating pathogenic bacteria, reducing the release of heterophilic antigens, and/or reducing heterophilic antibodies in the circulation.

### Example 6

### Application of Combined Antibodies in Animal Reproduction Models

Animal data demonstrated that a vaccine which combined RagB protein and Cra4S1 protein resulted in better local tissue protection effect than a single protein vaccine. However, the combined vaccine did not show cross protection to other subtypes of P. gingivalis. For example, animals immunized with a vaccine consisting of a mixture of RagB-1, RagB-3 and Cra4S1 were protected against subtype RagB-1 and RagB-3 of P. gingivalis infection, but not protective against the subtype P. gingivalis RagB-2 infection. Passive immunization confirmed that the combination of antibodies, e.g., mixture of anti-RagB and anti-Cra4S1, presented the best protective effect compared with any antibody alone and a control group. The protection details were described in PCT/CN2019/124433, which is incorporated by reference in its entity herewith.

The conventional antibody drug treatment was focused on the use of specific antibodies to provide a precise treatment based on the diagnosis of the dominant P. gingivalis strain of outer membrane protein RagB. As described in above Example 3, monoclonal antibodies against the four different RagB proteins and the Cra4S1 protein were identified and characterized.

A combination of multiple antibodies was designed. In this Example, 1D2-2-1-3 monoclonal antibody (specific to RagB-3) and Cra4S1 polyclonal antibody (from mouse serum) were combined. Specifically, male Balb/c mice aged 5-6 weeks old, weighing 15-18 g, six (6) were in each group. Four days before and one day before the bacterial infection, 100 μl antibody solution containing 50 μg monoclonal antibody, 200 μg mouse serum (Pab is mouse polyclonal antibody in Table 32) or a mixture of 50 μg monoclonal antibody and 200 μg mouse serum was injected into the abdominal cavity of the mice (Table 32), and the bacterial challenge was carried out.

Bacterial suspension preparation is briefly described herein: P. gingivalis, including various subtypes (P, gingivalis RagB-1, RagB-2, RagB-3 and RagB-4), were all grown on the Fastidious Anaerobic Agar (FAA) plate containing 5% defibrinated horse blood and placed in an anaerobic incubator at 37 °C, containing 80% nitrogen, 10% hydrogen and 10% carbon dioxide. The bacterial colonies were transferred to the freshly prepared Brain Heart extract (BHI) culture medium (containing 5 μg/ml hemin), grown for 18-24 hours, until the OD₆₀₀ nm reached 1-1.2. The culture was then centrifuged and washed twice, and following the washing, bacterial suspensions were created at different concentrations (2-8×10¹⁰ CFU/ml) using BHI culture medium for animal experiments.

TABLE 32
Passive Immunization Animal Information

| Group | Group | | Dose | Sex, | Bacterial |
|---|---|---|---|---|---|
| Name | Size | Antibody | μg/mouse | Age, Weight | Challenge |
| G1 | 6 | Mab 1D2-2-1-3 | 50 | Male, | P. gingivalis |
| G2 | 6 | Mab 1D2-2-1-3 | 50 | 5-6 weeks old, | RagB-3 |
| | | Anti-Cra4S1(Pab) | 200 | 15-18 g | |
| G3 | 6 | Anti-Cra4S1(Pab) | 200 | | |
| G4 | 6 | Normal Serum | 200 | | |
| G5 | 6 | Control | N/A | | |

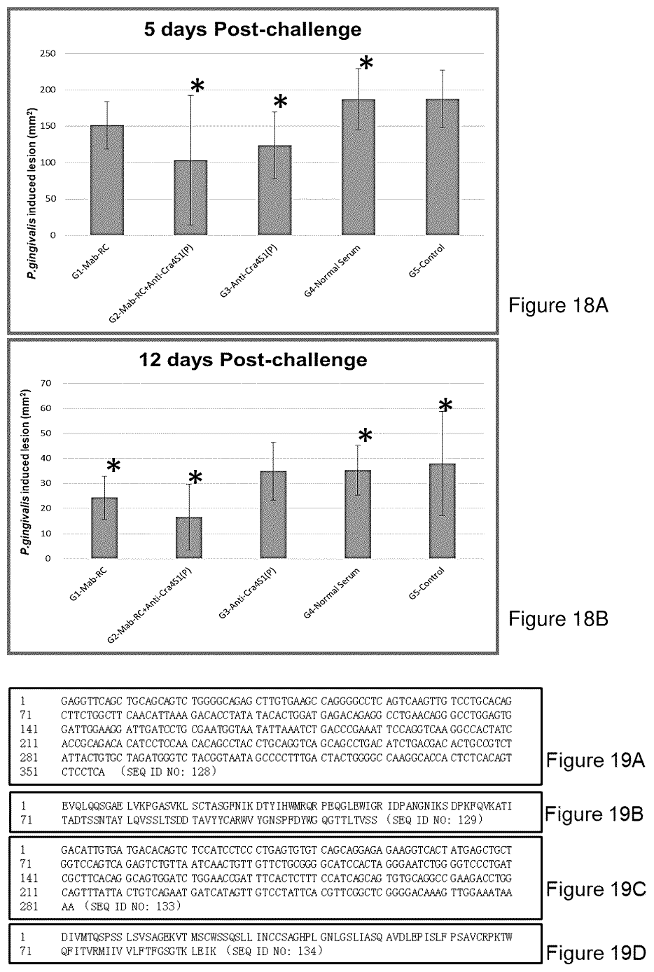

After the bacterial infection, the general condition of the animals and the development of local lesions were observed and photographs were taken, and the records are as shown in Table 33 and Table 34 and FIGS. 17A-17E and FIGS. 18A-18B.

The data showed that the animals that received a combination of RagB-3 monoclonal antibody 1D2-2-1-3- and Cra4S1 polyclonal antibodies achieved better local immune protection at the site of bacterial infection, with milder soft tissue damage and faster healing as compared with other groups of animals that did not receive the combined antibodies. The data also showed that systemic administration of the combination of the antibodies achieved local effects in the body's humoral circulation. In this case, intraperitoneally injection (a systemic administration technique) resulted in subcutaneous protection at the local injection site for subsequent P. gingivalis bacterial infection.

TABLE 33
Data Record for Immune Protective
Effect of Monoclonal Antibody

| Group | Indivadual | Local Lesion area (mm²) | | | |
|---|---|---|---|---|---|
| Number | Mouse | Day 5 | Day 8 | Day 12 | Day 17 |
| G1 | G1-1 | 199.37 | 139.04 | 24.48 | 0.00 |
| | G1-2 | 143.57 | 72.03 | 17.23 | 0.00 |
| | G1-3 | 141.90 | 91.81 | 19.95 | 0.00 |
| | G1-4 | 180.99 | 138.49 | 40.95 | 0.00 |
| | G1-5 | 128.95 | 66.16 | 23.88 | 0.00 |
| | G1-6 | 112.76 | 65.80 | 19.18 | 0.00 |
| G2 | G2-1 | 0.00 | 0.00 | 0.00 | 0.00 |
| | G2-2 | 159.49 | 71.14 | 27.22 | 0.00 |
| | G2-3 | 224.81 | 92.40 | 27.11 | 0.00 |
| | G2-4 | 107.67 | 89.02 | 26.44 | 0.00 |
| | G2-5 | 124.93 | 52.90 | 18.38 | 0.00 |
| | G2-6 | 0.00 | 0.00 | 0.00 | 0.00 |
| G3 | G3-1 | 199.43 | 108.33 | 34.63 | 4.80 |
| | G3-2 | 58.54 | 27.13 | 52.59 | 58.51 |
| | G3-3 | 119.52 | 83.04 | 33.63 | 10.14 |
| | G3-4 | 111.50 | 93.08 | 41.87 | 0.00 |
| | G3-5 | 139.79 | 41.08 | 27.86 | 0.00 |
| | G3-6 | 112.21 | 69.85 | 18.93 | 13.93 |
| G4 | G4-1 | 194.80 | 56.97 | 26.92 | 0.90 |
| | G4-2 | 121.92 | 41.90 | 27.61 | 0.00 |
| | G4-3 | 200.33 | 118.61 | 46.14 | 25.25 |
| | G4-4 | 250.55 | 96.50 | 49.46 | 8.73 |
| | G4-5 | 177.02 | 64.32 | 29.02 | 0.00 |
| | G4-6 | 178.11 | 101.76 | 32.42 | 14.22 |
| G5 | G5-1 | 176.63 | 181.22 | 30.82 | 0.00 |
| | G5-2 | 211.01 | 110.75 | 37.20 | 0.00 |
| | G5-3 | 242.07 | 100.55 | 27.44 | 0.00 |
| | G5-4 | 123.27 | 63.87 | 11.48 | 0.00 |
| | G5-5 | 180.47 | 121.93 | 72.89 | 0.00 |
| | G5-6 | 190.27 | 108.07 | 47.34 | 0.00 |

TABLE 34
Data Summary

Average Local Lesion area (mm²)

| | Day 5 | Day 8 | Day 12 | Day 17 |
|---|---|---|---|---|
| G1 | 151.26 | 95.56 | 24.28 | 0.00 |
| G2 | 102.82 | 50.91 | 16.52 | 0.00 |
| G3 | 123.50 | 70.42 | 34.92 | 14.56 |
| G4 | 187.12 | 80.01 | 35.26 | 8.18 |
| G5 | 187.28 | 114.40 | 37.86 | 0.00 |

Periodontal disease caused by P. gingivalis manifests as visible localized damages. A clinician working in periodontal clinics can determine the severity of periodontal disease by detecting the depth of the periodontal pockets and the susceptibility to bleeding gums. However, P. gingivalis infection is associated with a range of systemic diseases, and periodontal disease may be just the tip of the iceberg of the dangers posed by P. gingivalis infections.

The current conventional animal models of P. gingivalis infection mainly focus on soft tissue destruction and alveolar bone absorption. The virulence of different strains of P. gingivalis is not consistent, local tissue damage caused by infection of P. gingivalis can be observed in almost all animals and depend on an infection dose. Generally, the higher concentration of bacteria infecting the animal, the greater degree of local tissue damage. In most cases, infected animals are self-heal, but some abscesses may reappear after the wound had initially healed. Animals usually do not have a high mortality rate if infected with bacteria only. The pathogenic mechanism of P. gingivalis has never been conclusively proven, and the scientific community has not successfully established an animal model related to P. gingivalis infection and systemic disease up to date.

Over the years, clinical epidemiological data have shown that P. gingivalis infection in pregnant mothers is associated with preterm birth and low birth weight infants. Inspired by these clinical findings, an animal model was established to investigate the consequences of P. gingivalis infection on mouse reproduction.

To establish such animal model, three groups were established: the first group with both male and female mice receiving the combination of the antibodies and P. gingivalis infection; the second group control male mice with females receiving the combination of the antibodies and P. gingivalis infection; and the third group male mice receiving the combination of the antibodies and P. gingivalis infection with control female mice. The antibodies received were from rabbit antiserum, according to different antibody combinations as detailed in Table 35. Two intraperitoneally injections were given, with an interval of 4 days between antibody injections. The animals were then infected with P. gingivalis. During the recovery period, male and female mice were randomly selected and paired from each group; with 1 male and 2 female mice per cage, except for mice receiving the PBS control group where 1 male was paired with 1 female per cage.

In this Example, the numbers of female control mice (i.e., not receiving antibodies, not receiving P. gingivalis infection) were limited, so slightly younger female mice (aged 11-12 weeks rather than 18-20 weeks) were used as detailed in Table 35. As a comparison, similar aged control female mice were paired with control males to show that numbers of expected offspring are naturally lower in 11-12 weeks old females.

The results showed that when both parents were injected the combination of the antibodies followed by infection of P. gingivalis, there was a low rate of liveborn pups and a high rate of stillbirths (Table 36). Conversely, when mice without injected antibodies who received infection of P. gingivalis reproduced (the PBS groups), they had an increased number of liveborn pups and no stillbirths compared to those who received the injected combination of the antibodies.

In Table 37, female mice receiving the combination of the antibodies and subsequent P. gingivalis infection were paired with normal control male mice. These results showed an increased number of live born mice as compared to those when both sets of parents were injected the combination antibodies and infection. A control group where female mice receiving PBS and P. gingivalis infection were also paired with normal control male mice. In fact, the average number of liveborn pups from females (51/13, 3.92 live born/female mouse on average) injected with the combination of the antibodies plus infection was slightly higher than the number from female PBS control mice. Although the data may not provide enough power for showing statistical significance, these results suggest that antibody administration plus P. gingivalis infection in male and female mice plays an important role in offspring reproduction.

In Table 38, male mice receiving the combination of the antibodies and subsequent P. gingivalis infection were paired with normal control female mice. The results showed an improved number of liveborn pups as compared to both sets of parents received the combination of the antibodies and P. gingivalis infection. A control group where male mice receiving PBS and P. gingivalis infection were also paired with normal control female mice. The average number of live born pups from males injected with the combination of the antibodies plus infection was comparable to the number from male PBS control mice. Interestingly, the pairings in Table 38 had lower rates of liveborn pups and survival compared to those in Table 37, and higher stillbirth rates. These data suggest that male infection status following antibody administration and infection showed a higher impact on live birth rates than that on females with infection status.

TABLE 35
Experimental Animal Information

| Group | Sex and | Antibody Injection | Bacterial |
|---|---|---|---|
| Name | Age | Purified Rabbit IgG μg/mouse | Challenge |
| G871 | Male, | 200 μg Rabbit anti-RagB-1 + | P. gingivalis |
| | 18-20 weeks | 200 μg Rabbit anti-Cra4S1 | RagB-1 |
| G872 | old | 200 μg Rabbit anti-RagB-3 + | |
| | | 200 μg Rabbit anti-Cra4S1 | |
| G873 | | 200 μg Rabbit anti-GST + | |
| | | 200 μg Rabbit anti-Cra4S1 | |
| G874 | | 200 μg Rabbit anti-Cra4S1 | |
| G875 | | 200 μg Rabbit anti-RagB-1 | |
| G876 | | 200 μg Rabbit anti-RagB-3 | |
| G877 | | 200 μg Rabbit anti-GST | |
| G878 | | N/A | |
| G861 | Female, | 200 μg Rabbit anti-RagB-1 + | P. gingivalis |
| | 18-20 weeks | 200 μg Rabbit anti-Cra4S1 | RagB-1 |
| G862 | old | 200 μg Rabbit anti-RagB-3 + | |
| | | 200 μg Rabbit anti-Cra4S1 | |
| G863 | | 200 μg Rabbit anti-GST + | |
| | | 200 μg Rabbit anti-Cra4S1 | |
| G864 | | 200 μg Rabbit anti-Cra4S1 | |
| G865 | | 200 μg Rabbit anti-RagB-1 | |
| G866 | | 200 μg Rabbit anti-RagB-3 | |
| G867 | | 200 μg Rabbit anti-GST | |
| G868 | | N/A | |
| Control Mice | | | |
| G879 | Male, | N/A | N/A |
| | 18-20 weeks | | |
| G869 | Female, | N/A | N/A |
| | 11-12 weeks | | |
| | old | | |

TABLE 36
Reproduction Experiments (Part One)-male & female
both receiving antibodies and infection challenge
(plus control receiving no antibodies)

| | | | Male + Female | Live | |
|---|---|---|---|---|---|
| | Male | Female | number/Cage | Born Pups | Stillborn |
| | G871 | G861 | 1 + 2 | 7 | 1 |
| | G872 | G862 | 1 + 2 | 7 | 0 |
| | G873 | G863 | 1 + 2 | 0 | 0 |
| | G874 | G864 | 1 + 2 | 0 | 0 |
| | G875 | G865 | 1 + 2 | 1 | 5 |
| | G876 | G866 | 1 + 2 | 0 | 0 |
| | G877 | G867 | 1 + 1 | 0 | 0 |
| Total | | | | 15 | 6 |
| | G878 | G868 | 1 + 1 | 4 | 0 |

TABLE 37
Reproduction Experiments (Part Two)-females receiving antibodies
and infection challenge (plus control receiving no antibodies),
paired with males receiving no antibody and no infection challenge

| | | | Male + Female | Live | |
|---|---|---|---|---|---|
| | Male | Female | number/Cage | Born Pups | Stillborn |
| | G879 | G861 | 1 + 2 | 6 | 0 |
| | G879 | G862 | 1 + 2 | 8 | 0 |
| | G879 | G863 | 1 + 2 | 12 | 0 |
| | G879 | G864 | 1 + 2 | 7 | 0 |
| | G879 | G865 | 1 + 2 | 8 | 2 |
| | G879 | G866 | 1 + 2 | 10 | 0 |
| | G879 | G867 | 1 + 1 | 0 | 0 |
| Total | | | | 51 | 2 |
| | G879 | G868 | 1 + 1 | 3 | 0 |

TABLE 38
Reproduction Experiments (Part Three)-males receiving antibodies and
infection challenge (plus control receiving no antibodies), paired
with females receiving no antibody and no infection challenge

| | | | Male + Female | Live | |
|---|---|---|---|---|---|
| | Male | Female | number/Cage | Born Pups | Stillborn |
| | G871 | G869 | 1 + 2 | 0 | 3 |
| | G872 | G869 | 1 + 2 | 0 | 0 |
| | G873 | G869 | 1 + 2 | 4 | 2 |
| | G874 | G869 | 1 + 2 | 7 | 0 |
| | G875 | G869 | 1 + 2 | 7 | 0 |
| | G876 | G869 | 1 + 2 | 4 | 0 |
| | G877 | G869 | 1 + 2 | 0 | 0 |
| Total | | | | 22 | 5 |
| | G878 | G869 | 1 + 1 | 4 | 0 |

TABLE 39
Immunoprotective Effects of Combined Monoclonal Antibodies

| | | | | | Survive |
|---|---|---|---|---|---|
| | Sex + | | Antibody (μg) | Bacterial | after |
| Group | Age | Number | 100 μl/mouse | challenge | Chanllenge |
| G941 | Male, | 8 | Normal mouse IgG (100) | P. gingivalis | 7 |
| G942 | 14 weeks | 8 | 1D2 + 1B11 (50 + 50) | RagB-3 | 6 |
| G943 | | 8 | 1C3 + 1B11 (50 + 50) | 3.2 × 10¹⁰ | 8 |
| G944 | | 8 | 1D2 + 1C3 (50 + 50) | CFU/ml 200 μl | 6 |
| G945 | | 8 | PBS | | 8 |
| G951 | Female, | 5 | Normal mouse IgG(100) | P. gingivalis | 5 |
| G952 | 14 weeks | 5 | 1D2 + 1B11 (50 + 50) | RagB-3 | 5 |
| G953 | | 5 | 1C3 + 1B11 (50 + 50) | 3.2 × 10¹⁰ | 5 |
| G954 | | 5 | 1D2 + 1C3 (50 + 50) | CFU/ml 200 μl | 5 |
| G955 | | 5 | PBS | | 5 |

Following these results, the next step was to evaluate the effect of inoculation with other RagB monoclonal antibodies on these mouse models. As disclosed in Example 4, three monoclonal antibodies showed potential as agents for vaccination against P. gingivalis due to cross reactivity to multiple subtypes of RagB. They are RagB-3-1D2-2-1-3 (abbreviated to 1D2), RagB-4-1B11-4-4 (abbreviated to 1B11) and RagB-4-1C3-7-8 (abbreviated to 1C3). Since a single antibody showed limited effect in providing passive immunity, as previously described in PCT/CN2019/124433, this Example is to evaluate the effects of passive immunity with a combination of two (or more) monoclonal antibodies disclosed herein.

The same procedures were performed for animals receiving a combination of antibodies and P. gingivalis infection. Briefly, two intraperitoneally injections were given, with an interval of 4 days between antibody injections. The animals were then infected with P. gingivalis and the survival rate and local damage were observed and recorded.

Table 39 shows passive immunization results of animals receiving a combination of monoclonal antibodies and then infected with P. gingivalis infection. Male and female mice were inoculated with a combination of two monoclonal antibodies as schemed in Table 39, with control groups receiving normal mouse IgG or PBS, then infected with P. gingivalis RagB-3 subtype. One mouse (1/8) receiving normal mouse IgG died after bacterial infection, and two mice (2/8) in groups G942 and G944 also died. No female mice died.

During the recovery period, male and female mice were paired; 1 male and 3 female mice per cage. In each breeding group, two female mice received an immune challenge, and one female received no antibodies or infection. Body weights of female mice were recorded, as well as the number of live and stillborn pups. When a female mouse lost weight suddenly and produced no live or stillborn pups, this may be a case of miscarriage. Fewer female mice were available compared to males, resulting in shortage of female mice in each group, therefore, some females were used from other studies that had a recorded history of antibody injections and P. gingivalis infection, and details are shown in Table 40.

TABLE 40
Immunological history of additional females
used in reproductive mouse model

| Group | | | |
|---|---|---|---|
| Name | Age | Antibody Injection | Bacterial Challenge |
| G951 | 14 weeks old | Normal mouse IgG(100) | P. gingivalis RagB-3 |
| G952 | | 1D2 + 1B11 (50 + 50) | P. gingivalis RagB-3 |
| G953 | | 1C3 + 1B11 (50 + 50) | P. gingivalis RagB-3 |
| G954 | | 1D2 + 1C3 (50 + 50) | P. gingivalis RagB-3 |
| G955 | | PBS | P. gingivalis RagB-3 |
| G001 | 14 weeks old | N/A | N/A |
| G911 | 26 weeks old | mouse IgG | P. gingivalis RagB-1 |
| G912 | | PBS | P. gingivalis RagB-1 |
| G913 | | 1A1 + 1B11(80 + 20) | P. gingivalis RagB-1 |
| G914 | | 1D2 + 1B11 (50 + 50) | P. gingivalis RagB-1 |
| G915 | | 1A4 + 1B11(80 + 20) | P. gingivalis RagB-1 |
| G931 | | mouse IgG | P. gingivalis RagB-1 + |
| | | | RagB-3 |
| G932 | | PBS | P. gingivalis RagB-1 + |
| | | | RagB-3 |
| G933 | | 1H11 + 1B11 (80 + 20) | P. gingivalis RagB-1 + |
| | | | RagB-3 |
| G934 | | 1D2 + 1B11 (50 + 50) | P. gingivalis RagB-1 + |
| | | | RagB-3 |

TABLE 41
Effect of Combined Monoclonal Antibodies on Reproduction (1) - males
receiving mouse IgG and infection, paired with females receiving
the IgG and infection, and no antibody/infection as control

| | | | | Suspected | No Evidence |
|---|---|---|---|---|---|
| Male | Female | Liveborn | Stillborn | Miscarriage | of Pregnancy |
| G941-1 | G951 | 5 | | | |
| | G001 | 3 | | | |
| | G931 | | | | X |
| G941-2 | G951 | | | | X |
| | G001 | | | | X |
| | G931 | | | | X |
| G941-3 | G951 | 4 | | | |
| | G001 | 0 | 2 | | |
| | G931 | | | | X |
| G941-4 | G951 | | | | X |
| | G001 | | | | X |
| | G931 | | | | X |
| G941-5 | G951 | 4 | | | |
| | G001 | 4 | | | |
| | G931 | 2 | | | |
| G941-6 | G001 | | | | X |
| | G911 | | | | X |
| | G931 | | | | X |
| G941-7 | G001 | | | | X |
| | G911 | | | | X |
| | G931 | | | | X |
| Total | | 22 | 2 | | |

Group G941/G951 (Table 41) and Group G945/G955 (Table 45) are control groups where normal mouse IgG and PBS were given, respectively. Neither of these animals received any antibodies with protective properties prior to be infected with P. gingivalis, and they represent the baseline expected reproductive success of animals recovered from infection. Similar numbers of liveborn pups were observed in both groups, but in Group G941/G951 a high rate of females that do not become pregnant at all in four cages (4/7) was observed, and in Group G945/G955 a high rate of stillbirths was observed.

TABLE 42
Effect of Combined Monoclonal Antibodies on Reproduction (2) -
males receiving a combination of MAbs 1B11 and 1D2 and infection,
paired with females receiving the combination of the antibodies
and infection, and no antibody/infection as control

| | | | | Suspected | No Evidence |
|---|---|---|---|---|---|
| Male | Female | Liveborn | Stillborn | Miscarriage | of Pregnancy |
| G942-1 | G952 | | | | X |
| | G001 | | | | X |
| | G933 | | | | X |
| G942-2 | G952 | | | | X |
| | G001 | | | | X |
| | G933 | | | | X |
| G942-3 | G952 | 0 | | X | |
| | G001 | 4 | 1 | | |
| | G934 | 0 | | X | X |
| G942-4 | G952 | | | | X |
| | G001 | | | | X |
| | G934 | | | | X |
| G942-5 | G952 | | | | X |
| | G001 | | | | X |
| | G933 | | | | X |
| G942-6 | G001 | | | | X |
| | G934 | | | | X |
| | G933 | | | | X |
| Total | | 4 | 1 | | |

In the G942/G952 group (Table 42), animals received a combination of 1D2 and 1B11 monoclonal antibodies prior to be infected with P. gingivalis. Mortality in male mice after bacterial infection was 25% (2/8) as previously described. The group's subsequent reproductive success was also greatly affected. Five of the six male mice did not cause the female mice, including the control female mice, pregnant, only one male mouse was able to produce offspring, one normal female in this group gave birth to four live-born pups and one stillborn, and the other two females had suspected miscarriages.

In the G943/G953 group (Table 43), animals received a combination of 1B11 and 1C3 monoclonal antibodies prior to be infected with P. gingivalis. All animals, male and female, were survived after being infected by P. gingivalis. Both males and females recovered well, although two males failed to cause females (including the control females) pregnant. Of the remaining six cages, 48 livebirths were observed to the 18 of females, and a low number of stillbirths (four) were recorded.

In the G944/G954 group (Table 44), animals received a combination of 1D2 and 1C3 monoclonal antibodies prior to be infected with P. gingivalis. As mentioned earlier, the male mice had a 25% mortality (2/8) following bacterial infection. Subsequent reproductive success is similar to the G945/G955 control group received PBS before infection. One male (out of 6 remaining) failed to cause any females (including control females) pregnant. A reasonable number of livebirths: 31 pups to 15 females was observed. A high number of stillbirths (ten) was also observed, and three females suspected miscarriages.

TABLE 43
Effect of Combined Monoclonal Antibodies on Reproduction (3) -
males receiving a combination of MAbs 1B11 and 1C3 and infection,
paired with females receiving the combination of the antibodies
and infection, and no antibody/infection as control

| | | | | Suspected | No Evidence |
|---|---|---|---|---|---|
| Male | Female | Liveborn | Stillborn | Miscarriage | of Pregnancy |
| G943-1 | G953 | | | | X |
| | G001 | | | | X |
| | G913 | | | | X |
| G943-2 | G953 | 4 | 1 | | |
| | G001 | 6 | 0 | | |
| | G913 | 2 | 1 | | |
| G943-3 | G953 | | | | X |
| | G001 | 5 | 0 | | |
| | G913 | | | | X |
| G943-4 | G953 | 5 | 0 | | |
| | G001 | | | | X |
| | G913 | | | | X |
| G943-5 | G953 | 1 | 1 | | |
| | G001 | 5 | | | |
| | G913 | 6 | 0 | | |
| G943-6 | G001 | 6 | 0 | | |
| | G934 | | | | X |
| | G913 | | | | X |
| G943-7 | G914 | | | | X |
| | G001 | | | | X |
| | G913 | | | | X |
| G943-8 | G001 | 3 | 0 | | |
| | G914 | | | | X |
| | G913 | 5 | 0 | | |
| Total | | 48 | 4 | | |

TABLE 44
Effect of Combined Monoclonal Antibodies on Reproduction (4) -
males receiving a combination of MAbs 1C3 and ID2 and infection,
paired with females receiving the combination of the antibodies
and infection, and no antibody/infection as control

| | | | | Suspected | No Evidence |
|---|---|---|---|---|---|
| Male | Female | Liveborn | Stillborn | Miscarriage | of Pregnancy |
| G944-1 | G954 | | | | X |
| | G001 | 3 | 2 | | |
| | G915 | 0 | 3 | | |
| G944-2 | G954 | 2 | 2 | | |
| | G001 | 8 | 0 | | |
| | G915 | 2 | 1 | | |
| G944-3 | G954 | | | | X |
| | G001 | 0 | 2 | | |
| | G915 | | | | X |
| G944-4 | G954 | | | | X |
| | G001 | | | | X |
| | G915 | | | | X |
| G944-5 | G954 | | | X | |
| | G001 | 5 | 0 | | |
| | G915 | 6 | 0 | | |
| G944-6 | G915 | | | X | |
| | G001 | 5 | 0 | | |
| | G915 | | | X | |
| Total | | 31 | 10 | | |

TABLE 45
Effect of Combined Monoclonal Antibodies on Reproduction (5) -
males receiving PBS and infection, paired with females receiving
PBS and infection, and no antibody/infection as control

| | | | | Suspected | No Evidence |
|---|---|---|---|---|---|
| Male | Female | Liveborn | Stillborn | Miscarriage | of Pregnancy |
| G945-1 | G955 | | | | X |
| | G001 | 0 | 7 | | |
| | G912 | | | | X |
| G945-2 | G955 | 4 | 2 | | |
| | G001 | 4 | 0 | | |
| | G912 | 0 | 2 | | |
| G945-3 | G955 | 3 | 0 | | |
| | G001 | | | | X |
| | G912 | 7 | 0 | | |
| G945-4 | G955 | | | | X |
| | G001 | 6 | 0 | | |
| | G912 | | | | X |
| G945-5 | G955 | | | | X |
| | G001 | 5 | 0 | | |
| | G912 | 5 | 0 | | |
| G945-6 | G001 | | | | X |
| | G932 | 3 | 1 | | |
| | G912 | 4 | 1 | | |
| G945-7 | G932 | | | | X |
| | G001 | | | | X |
| | G912 | | | | X |
| G945-8 | G001 | 5 | 3 | | |
| | G914 | | | | X |
| | G914 | 2 | 1 | | |
| Total | | 48 | 17 | | |

It is worth noting that the Rag 1D2 monoclonal antibody, in animal studies (shown in Table 33 and Table 34), showed local protective effects when the animal were infected with P. gingivalis. Furthermore, in multiple ELISA studies, the 1D2 showed high-affinity antigen-antibody reactions. However, the effects on animal reproduction following 1D2 inoculation (in various combinations) may cause safety concerns, as a 25% mortality among male mice in both group G942 and G944, a decrease in livebirth rate, increase in miscarriage and stillbirth rate, and a decrease in successful fertilization or a combination of these indicators, were observed.

The results from the animal models disclosed herein demonstrated that P. gingivalis infection causes reproductive damages. Literature has reported that pregnant women with periodontal disease are 3-7 times more likely to give birth to premature infants and infants of low birth weight compared to pregnant women without periodontal disease. For many years, researchers have focused on the maternal reproductive system and features of the fetus, studying genetics, endocrine features, and tissue and organ infections.

This Example shows that P. gingivalis infection caused reproductive damage. When both parents were infected, it results in the poorest outcomes with increased rates of failed fertilization, high stillbirth, and high miscarriage. The data disclosed in this Example also shows that when infected females were paired with uninfected males, their reproductive status were maintained, but conversely, when an infected male was paired with an uninfected female their outcomes were poorer. These results clearly show that paternal P. gingivalis infection and immune status has a detrimental effect as infected males always had poorer reproductive outcomes in these animal models. A possible mechanism of action is through heterophilic antigens present on the surface of P. gingivalis, which have similarities to host tissues, such as spermatozoa, or tissues of the male reproductive system.

Further, P. gingivalis specific antibodies can provide an immune protective function but can also cause immune mediated pathological damages. For example, 1D2 monoclonal antibody has a strong antigen-antibody response to multiple RagB subtypes, reacting with RA-17, RB-17 and RC-17 peptide targets (Example 4) and showed local immune protection, however, it has been noted that this monoclonal antibody also caused mortality and reduced reproductive success in this Example.

The data also disclosed in this Example showed that two other monoclonal antibodies, both specific to RagB-4, 1B11-4-4 (1B11) and RagB-4-1C3-7-8 (1C3), are capable of cross-reacting to multiple RagB subtypes and are safety and do not cause harm to the animal reproduction system, the combined antibodies effectively improve the physical condition of male and female animals, and directly or indirectly restore/cure reproductive success. The data suggested that these combined antibodies could be used for other conditions and/or systemic diseases associated/related to P. gingivalis infection, including but not limited to, periodontal disease, cardiovascular disease, rheumatoid arthritis, oral gastrointestinal cancer, ulcerative colitis, neurological disorders, autoimmune encephalomyelitis, lung cancer, adverse pregnancy, pancreatic cancer, diabetes, chronic kidney disease, bacterial pneumonia and chronic obstructive pulmonary disease. One of the treatment strategies would be to remove harmful antigens and/or antibodies involved in the occurrence and development of diseases, and/or to improve the titer or concentration of beneficial antibodies in the body.

### Example 7

### Mouse Monoclonal Antibodies and Humanization

Four monoclonal antibody hybridoma cell lines: RagB-2-1A4-3-7-7, RagB-3-1D2-2-1-3, RagB-4-1B11-4-4 and RagB-4-1C3-7-8, all showed cross-reacting with different subtypes of P. gingivalis outer membrane proteins. To sequence each, total RNA of hybridoma cell lines was first extracted and reverse-transcribed with antisense primers according to the conventional protocol for RNA extraction and transcription. Next, amplification of the light chain and heavy chain variable region sequences and the PCR fragments placed into the pUC19T vector. Colony PCR screening was then performed, and positive clones were chosen for sequencing.

The nucleotide and amino acid sequences of each idiotypic antibodies are provided as follows:

RagB-2-1A4-3-7-7 (Biointron, China, B467701, aka “1A4”) comprises a heavy chain (Mouse IgG1) variable region (V_{H}) comprising a nucleotide sequence as set forth in SEQ ID NO: 128 that encodes an amino acid sequence as set forth in SEQ ID NO: 129; and a light chain (Mouse Kappa) variable region (V_{L}) comprising a nucleotide sequence as set forth in SEQ ID NO: 133 that encodes an amino acid sequence as set forth in SEQ ID NO: 134 (see also FIGS. 19A-19D). The expression of recombinant antibody is not provided.

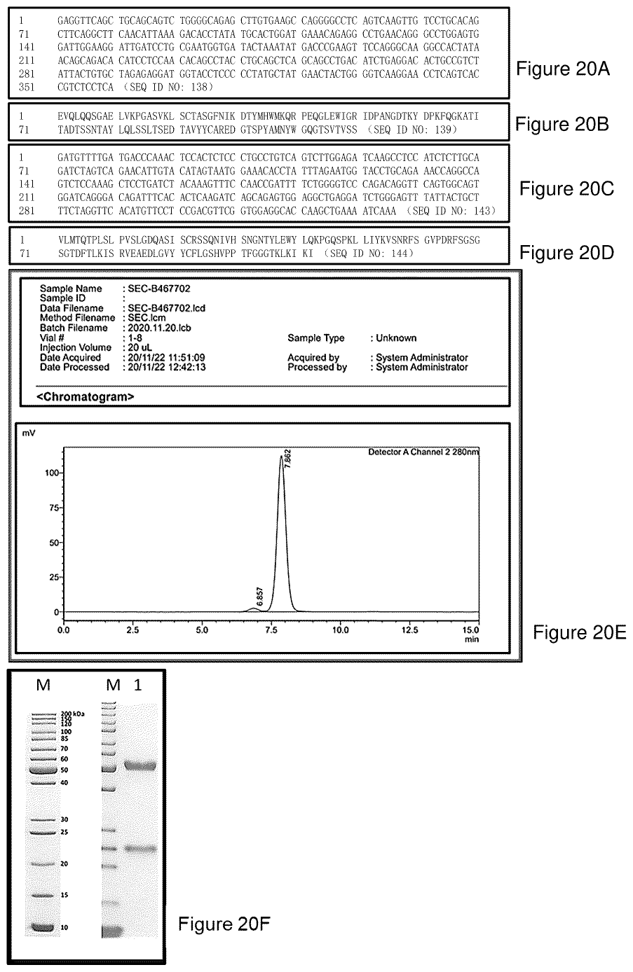

RagB-3-1D2-2-1-3 (Biointron, China, B467702, aka “1D2”) comprises a heavy chain (Mouse IgG2a) variable region (V_{H}) comprising a nucleotide sequence as set forth in SEQ ID NO: 138 that encodes an amino acid sequence as set forth in SEQ ID NO: 139, and a light chain (Mouse Kappa) variable region (V_{L}) comprising a nucleotide sequence as set forth in SEQ ID NO: 143 that encodes an amino acid sequence as set forth in SEQ ID NO: 144 (see also FIGS. 20A-20D). The expression of recombinant antibody was shown in FIGS. 20E and 20F.

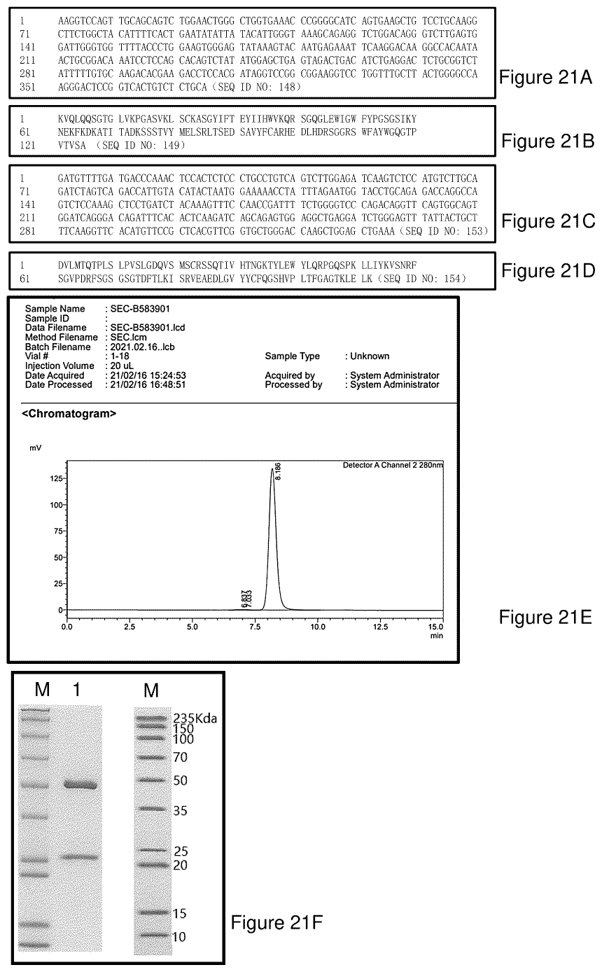

Ragb-4-1B11-4-4 (Biointron, China, B583901, aka “1B11”) comprises a heavy chain (Mouse IgG1) variable region (V_{H}) comprising a nucleotide sequence as set forth in SEQ ID NO: 148 that encodes an amino acid sequence as set forth in SEQ ID NO: 149, and a light chain (Mouse Kappa) variable region (V_{L}) comprising a nucleotide sequence as set forth in SEQ ID NO: 153 that encodes an amino acid sequence as set forth in SEQ ID NO: 154 (see also FIGS. 21A-21D). Bispecific antibodies of this monoclonal antibody are also provided in the present disclosure. The expression of recombinant antibody was shown in FIGS. 21E and 21F

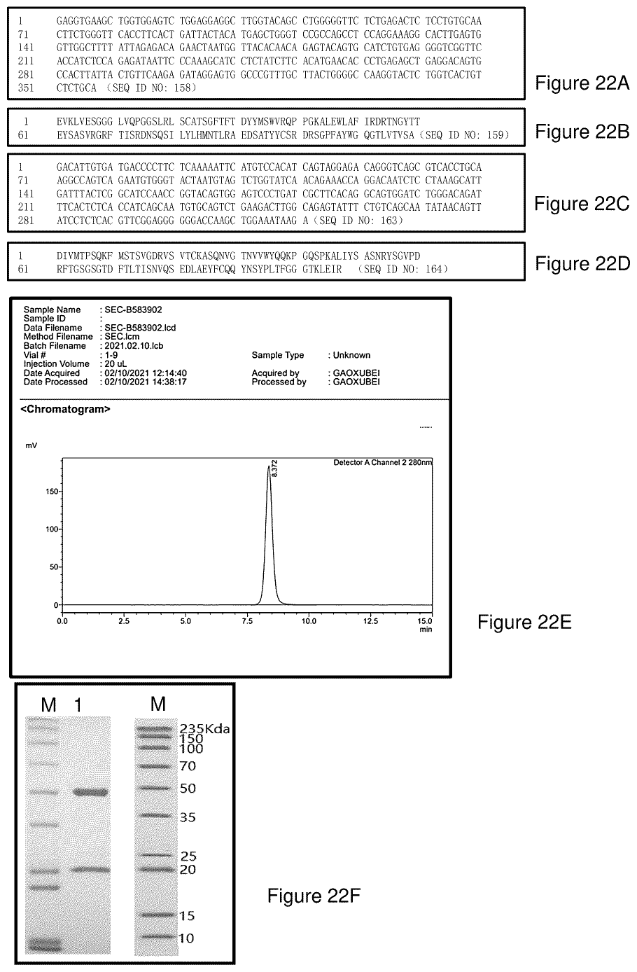

RagB-4-1C3-7-8 (Biointron, China, B583902, aka “1C3”) comprises a heavy chain (Mouse IgG1) variable region (V_{H}) comprising a nucleotide sequence as set forth in SEQ ID NO: 158 that encodes an amino acid sequence as set forth in SEQ ID NO: 159, and a light chain (Mouse Kappa) variable region (V_{L}) comprising a nucleotide sequence as set forth in SEQ ID NO: 163 that encodes an amino acid sequence as set forth in SEQ ID NO: 164 (see also FIGS. 22A-22D). The present disclosure provides detailed descriptions of this monoclonal and bispecific antibodies thereof. The expression of recombinant antibody was shown in FIGS. 22E and 22F.

TABLE 46
Physical and Chemical Properties of Recombinant Antibodies

| | | | Expected MW (kDa) | | | Antibody Purity | | |
|---|---|---|---|---|---|---|---|---|
| Contract | | | Reduction | Non | Expression | SEC | | Endotoxin |
| Number | Antibody Name | Ig subclass | Heave/Light | Reduction | Rate | 280 nm | SDS-PAGE | EU/mg |
| B467702 | RagB-3-1D2-2-1-3 | Mouse IgG2a-Kappa | 49/24 Kda | 146 Kda | 30.1 mg/L | 97.5 | >95 | <1 |
| B583901 | RagB-4-1B11-4-4 | Mouse IgG1-Kappa | 50/24 Kda | 148 Kda | 66.7 | 99.305 | >95 | <1 |
| B583902 | RagB-4-1C3-7-8 | Mouse IgG1-Kappa | 49/24 Kda | 146 Kda | 44.4 | 100 | >95 | <1 |

The recombinant plasmids of above three RagB monoclonal antibodies include 1D2, 1B11 and 1C3 were expressed in eukaryotic cells HEK293 by CRO Company (Biointron, China). The purification results of the recombinant antibodies are shown in FIGS. 20E-20F, 21E-21F, and 22E-22F, and the physical and chemical properties of each recombinant antibody are shown in Table 46. The ELISA method was used to detect the binding function of recombinant antibodies to antigen (Table 47).

Table 47 shows the detection results of recombinant antibody plasmids. The ELISA operation steps are as described in Example 2, and the antigen coating design is described as follows: the first column: RA-21 peptide, the second column: RB-21 peptide, the third column: RC-21 peptide, the fourth column: RC-22 peptide, the fifth column: RD-21 peptide, the sixth column: RD-22 peptide, the seventh column: RagB-1 recombinant protein, the eighth column: RagB-2 recombinant protein, the ninth column: RagB-3 recombinant protein, the tenth column: RagB-4 recombinant protein, the eleventh column: Cra4S1 recombinant protein, and the twelfth column: RC-17 peptide. The antibody dilution was 100 μg/ml, each antibody was added to one row of the ELISA plate, and there were 12-well antigen detections, including RA-21 (SEQ ID NO. 31), RB-21 (SEQ ID NO. 55), RC-21 (SEQ ID NO. 79), RC-22 (SEQ ID NO. 80), RD-21 (SEQ ID NO. 103), RD-22 (SEQ ID NO. 104), RagB-1 (SEQ ID NO. 2), RagB-2 (SEQ ID NO. 4), RagB-3 (SEQ ID NO. 6), RagB-4 (SEQ ID NO. 8), Cra4S1 (SEQ ID NO. 10), and RC-17 (SEQ ID NO. 75).

TABLE 47
Monoclonal Antibody Peptide Target Detection

| | | 210606 | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Antibody | Peptide | RA-21 | RB-21 | RC-21 | RC-22 | RD-21 | RD-22 | RagB-1 | RagB-2 | RagB-3 | RagB-4 | Cra4S1 | RC-17 |
| Bispecific 1C3-1B11 | A | 0.349 | 1.279 | 0.239 | 0.724 | 1.402 | 0.265 | 1.000 | 0.795 | 0.849 | 1.045 | 0.237 | 0.196 |
| Mixure of Recombinant | B | 0.488 | 1.034 | 0.694 | 0.503 | 1.171 | 0.253 | 0.875 | 0.774 | 0.893 | 1.263 | 0.233 | 0.213 |
| (1C3-7-8 + 1B11-4-4) | | | | | | | | | | | | | |
| Recombinant 1C3-7-8 | C | 0.154 | 1.028 | 0.151 | 0.827 | 1.107 | 0.154 | 0.771 | 0.409 | 0.778 | 1.026 | 0.152 | 0.147 |
| Recombinant 1B11-4-4 | D | 0.520 | 0.292 | 0.731 | 0.284 | 0.571 | 0.293 | 0.933 | 0.876 | 0.786 | 1.228 | 0.248 | 0.222 |
| Recombinant 1D2-2-1-3 | E | 0.144 | 0.157 | 0.155 | 0.155 | 0.153 | 0.156 | 1.415 | 0.976 | 1.544 | 0.159 | 0.153 | 1.581 |
| 1C3-7-8-ascites | F | 0.158 | 1.194 | 0.159 | 0.741 | 1.232 | 0.158 | 0.906 | 0.433 | 0.863 | 1.099 | 0.151 | 0.143 |
| 1B11-4-4-ascites | G | 0.625 | 0.362 | 0.858 | 0.291 | 0.677 | 0.445 | 0.894 | 0.891 | 0.853 | 1.229 | 0.305 | 0.277 |
| Normal mouse serum | H | 0.144 | 0.159 | 0.157 | 0.154 | 0.160 | 0.157 | 0.202 | 0.204 | 0.188 | 0.207 | 0.148 | 0.147 |

The data showed that the recombinant antibodies showed the same pattern of binding reaction to their corresponding antigens as compared to each corresponding monoclonal antibody produced by hybridoma cells. Under the conditions of the same dilution of the antibody, the absorbance value and pattern of the ELISA reaction plate are very similar, indicating that the recombinant antibody retains the antigen-binding function and the stability of the original monoclonal antibody. These recombinant antibodies are candidates for further development to become therapeutic agents.

Example 6 discloses that two monoclonal antibodies 1B11-4-4 (1B11) and 1C3-7-8 (1C3) were combined and showed cross-immune protection against four different subtypes of P. gingivalis. The 1B11-4-4 (1B11) antibody also cross-reacts with the Cra4S1 protein (Table 47), a conserved epitope in all subtypes of P. gingivalis. Since Cra4S1 is considered a heterophilic antigen, monoclonal antibody 1B11 can synergize by reducing the heterophilic antigen of Cra4S1, in addition to its cross-immunoprotection against other P. gingivalis outer membrane protein isoforms.

Therefore, humanized monoclonal antibodies of RagB-4-1B11-4-4 (1B11) and RagB-4-1C3-7-8 (1C3) are conducted according to conventional methods by the third-party CRO Company (Genscript, USA). The present disclosure provides the analysis of the mouse chimeric antibody, humanized gene synthesis, antibody expression and affinity sorting, antibody production and seed cell bank establishment to achieve antibody industrialization of production.

The MAbs disclosed herein are prepared to treat chronic diseases caused by P. gingivalis infection, including but not limited to periodontal disease, cardiovascular disease, rheumatoid arthritis, oral gastrointestinal cancer, ulcerative colitis, neurological disorders, autoimmune encephalomyelitis, lung cancer, adverse pregnancy, pancreatic cancer, diabetes, chronic kidney disease, bacterial pneumonia and chronic obstructive pulmonary disease.

### Example 8

### RagB Bispecific Monoclonal Antibody

A bispecific monoclonal antibody (BsMAb) is an artificially made protein that can simultaneously bind to two different antigens or two and more different epitopes. Naturally occurring monoclonal antibodies typically target only one antigen. Upon development, BsMAb can be manufactured in several structural formats. Through different mechanisms of action, BsMAb can be designed to recruit and activate immune cells, to interfere with receptor signalling and inactivate signalling ligands, and to be associated with protein complexes.

The data presented above showed that the antigenic targets of the two monoclonal antibodies RagB-4-1B11-4-4 (1B11) and RagB-4-1C3-7-8 (1C3) are two adjacent epitopes within the region of peptides RD-21&22 of the P. gingivalis outer membrane protein RagB-4, and the combination of two monoclonal antibodies showed stable systemic and local immune protective effects. Therefore, bispecific monoclonal antibody 1B11-1C3 (Biointron, China, B745901) combining RagB-4-1B11-4-4 (1B11) and RagB-4-1C3-7-8 (1C3) was conducted, which comprises a heavy chain comprising a nucleotide sequence as set forth in SEQ ID NO: 168 that encodes an amino acid sequence as set forth in SEQ ID NO:172, and a light chain comprising a nucleotide sequence as set forth in SEQ ID NO:173 that encodes an amino acid sequence as set forth in SEQ ID NO:174.

Specifically, the bispecific antibody 1B11-1C3 used scFv mode, a single-chain variable fragment (scFv), is a fusion protein of the variable regions of the heavy (V_{H}) and light chains (V_{L}) of immunoglobulins, connected with a commercial available short linker peptide. The fusion protein comprises heavy chain comprising variable region of specific monoclonal antibodies connected by short linker peptide and IgG1 heavy chain constant region (CH), Mus musculus albula; light chain comprising variable region of specific monoclonal antibody and an immunoglobulin kappa constant, Mus musculus albula.

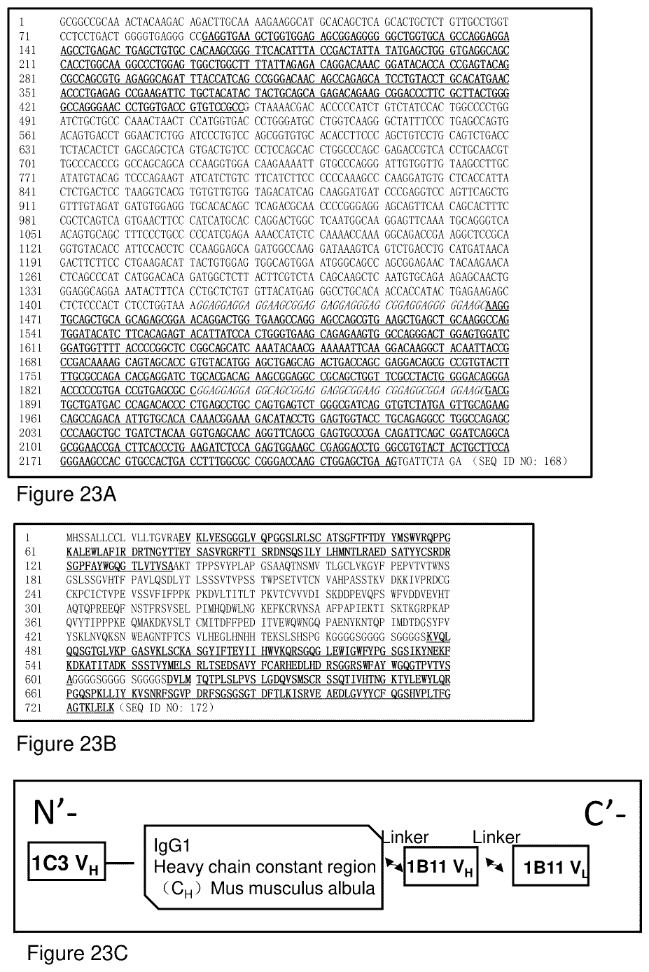

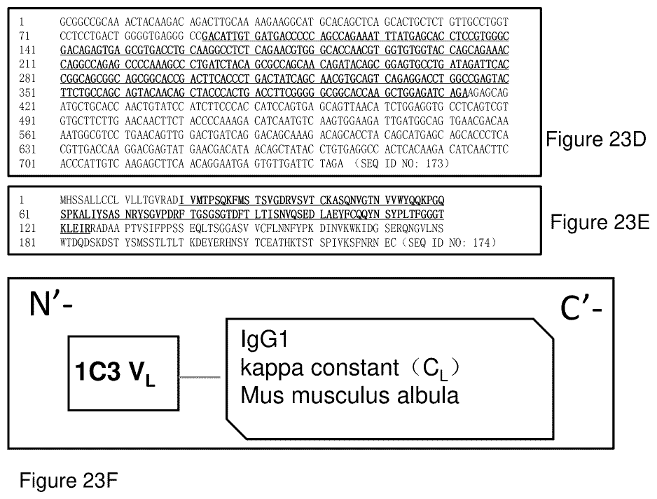

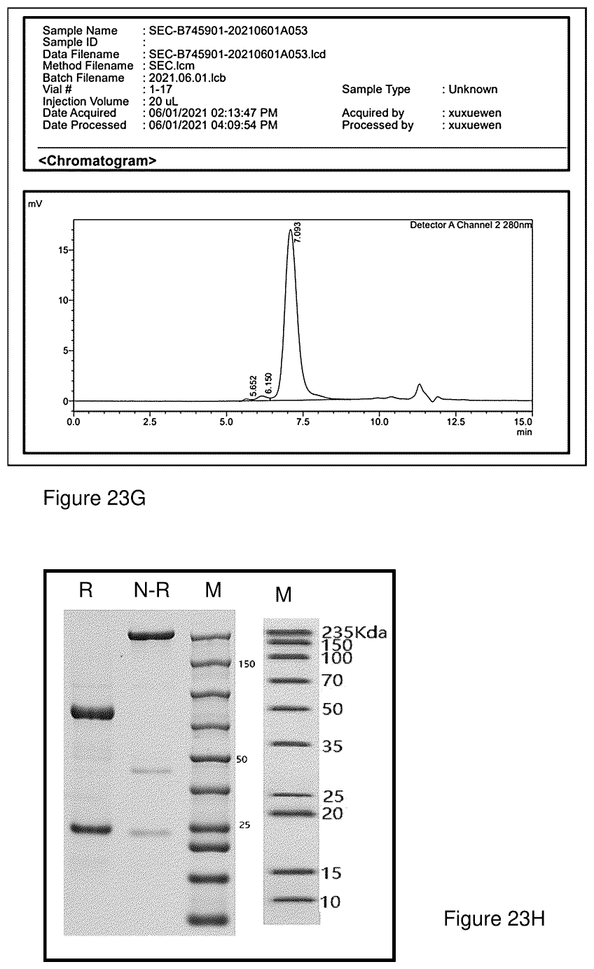

The heavy chain of bispecific 1B11-1C3 antibody comprises a heavy chain variable region V_{H} of nucleotide sequences as set forth in SEQ ID NO: 169 (1C3 V_{H}), SEQ ID NO: 170 (1B11 V_{H}), and SEQ ID NO: 171 (1B11 V_{L}), that encode amino acid sequences as set forth in SEQ ID NO: 159 (1C3 V_{H}), SEQ ID NO: 149 (1B11 V_{H}) and SEQ ID NO: 154 (1B11 V_{L}) respectively; a light chain variable region V_{L} of a nucleotide sequence as set forth in SEQ ID NO: 173, that encode an amino acid sequence of 1C3 full length light chain variable region as set forth in SEQ ID NO: 164 (see also FIGS. 23A-23F).

TABLE 48
Physical and Chemical Properties of Recombinant Bispecific Antibody

| | | | Expected MW (kDa) | | | Antibody Purity | | |
|---|---|---|---|---|---|---|---|---|
| CRO company | Antibody | | Reduction | Non | Expression | SEC | | Endotoxin |
| Contract Number | Name | Ig subclass | Heave/Light | Reduction | Rate | 280 nm | SDS-PAGE | EU/mg |
| Biointron, China | 1B11-1C3 | IgG1-Kappa, | 76/24 Kda | 200 Kda | 5.1 mg/L | 97.5 | >95 | <1 |
| B745901 | | Recombinant Bispecific | | | | | | |
| | | Antibody | | | | | | |

The recombinant plasmid was transformed into eukaryotic cells HEK293, and the recombinant antibodies were induced to be expressed and purified by Protein A affinity chromatography (FIGS. 23G and 23H). The physical and chemical properties of the recombinant bispecific antibody protein are shown in Table 48. The ELISA method was used to detect antigen-antibody responses; Table 47 in Example 7 also provided the data of the bispecific antibodies. The experimental procedure of ELISA is as described in Example 2, and the design of antigen coating is as described in Example 7. The antibody dilution was 100 μg/ml, and each antibody had a 12-well antigen detection.

The data presented herein show that the bispecific 1B11-1C3 showed antigen-antibody binding reactions to each corresponding target peptide fragment, and recombinant protein of different subtypes, as each individual monoclonal antibody does to its corresponding peptide target. The bispecific antibody also showed the same effect as a combination of each monoclonal antibodies 1B11-4-4 and 1C3-7-8 does. Compared with combining antibodies and single antibody use, the fusion protein retained the specificity of the original immunoglobulin, and enhance the immune response to antigen targets. The BsMAb disclosed herein is prepared to treat chronic diseases caused by P. gingivalis infection, including but not limited to periodontal disease, cardiovascular disease, rheumatoid arthritis, oral gastrointestinal cancer, ulcerative colitis, neurological disorders, autoimmune encephalomyelitis, lung cancer, adverse pregnancy, pancreatic cancer, diabetes, chronic kidney disease, bacterial pneumonia and chronic obstructive pulmonary disease.

In terms of ensuring efficacy and achieving a stable yield, bispecific antibodies represent a more efficient and simpler solution in developing new therapeutics.

### Example 9

### Serum Antibody Diagnostic Kit

The data presented in Example 5 show that about 40-50% of patients with cardiovascular disease and geriatric patients have high titer antibodies against P. gingivalis outer membrane proteins RagB and Cra4S1, and to irrelevant proteins (e.g., GST recombinant protein). These data suggest that qualitative and/or quantitative detection and/or measurement of these antibodies could provide early diagnosis of P. gingivalis infection; evaluate risk of developing a chronic disease associated with P. gingivalis infection; and assess disease prognosis and treatment effectiveness.

Therefore, the present disclosure provides a method of diagnosing or assessing prognosis and/or treatment effectiveness for P. gingivalis infection or a chronic disease resulted from or associated with P. gingivalis using the antibodies disclosed herein. Specifically, patients can be screened for antibodies to RagB proteins (including Rag-1, RagB-2, RagB-3, and RagB-4), Cra4S1 proteins and unrelated proteins (including GST and/or other tissue proteins such as muscle proteins or mucosal proteins). The ELISA technique is used to show their presence or absence in the sera of individual patients. If a high number of antibodies are present, and especially at high titers, it suggests that the patient is in a state of immune sensitization, which is at a high risk for potentially developing certain chronic diseases. This screening allows stratification of patients and identification of at-risk individuals, and prompts rapid commencement of treatment.

### Example 10

### PgingiVacRD1B11-1C3, Polypeptide Vaccine

Animal model test data showed that most animals with local damage caused by initial P. gingivalis infection can self-heal, but if the infection is repeated, their conditions could gradually deteriorate, and the local damage becomes difficult to heal. These phenomena indicated that host immune protection and bacterial pathology enter a process of continuous imbalance with repair then deterioration, and chronic P. gingivalis infection is never fully eradicated.

Certain diseases require patients to take antibody drugs for long term treatment often without any interruption. However, a one-off treatment with a vaccination could represent a more effective, easy and cost-effective way for a treatment.

For the treatment of P. gingivalis infection, after a period of passive immunotherapy with specific antibodies, the patient could be vaccinated with a high-efficiency polypeptide vaccine to stimulate his/her own active immune to generate effective neutralizing antibodies which remain in the patient's sera for long term in preventing the growth and spread of pathogenic bacteria. The polypeptide vaccine disclosed herein is cost-effective and can be administered safely via a conventional route. Moreover, vaccine products do not contain a full-length protein, can particularly avoid harmful, allergic and unrelated antigenic fragment so as to reduce the risk of heterophilic antigens stimulating heterophilic antibodies.

The present disclosure provides a polypeptide vaccine, namely, PgingiVacRD1B11-1C3 that is based on the RD21 to RD22 peptide fragment that RagB-4-1B11-4-4 (1B11) and/or RagB-4-1C3-7-8 (1C3) antibodies react to. As part of the process of refinement, both ends of the polypeptide were appropriately optimized. The polypeptide of PgingiVacRD1B11-1C3 vaccine can be synthesize and recombinant expression plasmids containing the polypeptide can be constructed and expressed in host cells inductive conditions. The protein expressed in the host cells can be purified by the conventional methods. The PgingiVacRD1B11-1C3 polypeptide comprises 75 amino acids, its amino acid sequence is set forth in SEQ ID NO. 127.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A monoclonal antibody that targets one or more outer membrane proteins of P. gingivalis, comprises:
(i) a light chain variable domain (V_{L}) comprising an amino acid sequence set forth in SEQ ID NO.154, which comprises (a) a CDR-L1 comprising an amino acid sequence set forth in SEQ ID NO.155; (b) a CDR-L2 comprising an amino acid sequence set forth in SEQ ID NO.156; and (c) a CDR-L3 comprising an amino acid sequence set forth in SEQ ID NO. 157, and a heavy chain variable domain (V_{H}) comprising an amino acid sequence set forth in SEQ ID NO.149, which comprises: (a) a CDR-H1 comprising an amino acid sequence set forth in SEQ ID NO.150; (b) a CDR-H2 comprising an amino acid sequence set forth in SEQ ID NO.151; and (c) a CDR-H3 comprising an amino acid sequence set forth in SEQ ID NO.152;
(ii) a light chain variable domain (V_{L}) comprising an amino acid sequence set forth in SEQ ID NO.164, which comprises (a) a CDR-L1 comprising an amino acid sequence set forth in SEQ ID NO.165; (b) a CDR-L2 comprising an amino acid sequence set forth in SEQ ID NO.166; and (c) a CDR-L3 comprising an amino acid sequence set forth in SEQ ID NO.167, and a heavy chain variable domain (V_{H}) comprising an amino acid sequence set forth in SEQ ID NO.159, which comprises: (a) a CDR-H1 comprising an amino acid sequence set forth in SEQ ID NO.160; (b) a CDR-H2 comprising an amino acid sequence set forth in SEQ ID NO. 161; and (c) a CDR-H3 comprising an amino acid sequence set forth in SEQ ID NO. 162; or
(iii) a light chain variable domain (V_{L}) comprising an amino acid sequence set forth in SEQ ID NO. 144, which comprises (a) a CDR-L1 comprising an amino acid sequence set forth in SEQ ID NO. 145; (b) a CDR-L2 comprising an amino acid sequence set forth in SEQ ID NO. 146; and (c) a CDR-L3 comprising an amino acid sequence set forth in SEQ ID NO. 147, and a heavy chain variable domain (V_{H}) comprising an amino acid sequence set forth in SEQ ID NO. 139, which comprises: (a) a CDR-H1 comprising an amino acid sequence set forth in SEQ ID NO. 140; (b) a CDR-H2 comprising an amino acid sequence set forth in SEQ ID NO. 141; and (c) a CDR-H3 comprising an amino acid sequence set forth in SEQ ID NO. 142.

2. The monoclonal antibody of claim 1, which is a Fab, Fab′, a F(ab)′2, a single-chain Fv(scFv), a Fv fragment or an Ig.

3. The monoclonal antibody of claim 1, is a diabody, a linear antibody or a bispecific or multispecific antibody.

4. (canceled)

5. An isolated nucleic acid molecule encoding the monoclonal antibody of claim 1.

6. An expression vector comprising the isolated nucleic acid molecule of claim 5.

7. A cell or a cell line comprising the isolated nucleic acid molecule of claim 6.

8.-11. (canceled)

12. A composition comprising the monoclonal antibody of claim 1, and a pharmaceutically acceptable carrier.

13. A composition comprising the isolated nucleic acid molecule of claim 5, and a pharmaceutically acceptable carrier.

14. A composition comprising the cell or cell line of claim 7, and a pharmaceutically acceptable carrier.

15. A method for treating a subject with an infection caused by P. gingivalis or a chronic disease resulted from P. gingivalis infection comprising administering to the subject an effective amount of the composition of claim 12.

16. The method of claim 15, wherein the chronic disease is periodontal disease, cardiovascular disease, rheumatoid arthritis, oral gastrointestinal cancer, ulcerative colitis, neurological disorders, autoimmune encephalomyelitis, lung cancer, adverse pregnancy, pancreatic cancer, diabetes, chronic kidney disease, bacterial pneumonia and chronic obstructive pulmonary disease.

17. A method of producing the monoclonal antibody of claim 1, comprising culturing the cell or the cell line comprising the isolated nucleic acid molecule encoding the monoclonal antibody and recovering the monoclonal antibody from cultured cell.

18. An antigen of an outer membrane protein of P. gingivalis that reacts or cross reacts with the monoclonal antibody of claim 1.

19. (canceled)

20. A vaccine comprising the antigens of claim 18.

21.-26. (canceled)

27. An animal model used to evaluate P. gingivalis infection and treatment efficacy and prognosis, wherein an animal is infected by P. gingivalis, and used for evaluating the monoclonal antibody of claim 1, antibody-antigen reaction or cross-reaction, and effects on animal's offspring.

28. A method of diagnosis and prognosis of an infection caused by P. gingivalis, comprising:
(a) identifying serum polyclonal antibodies against one or more outer membrane protein of P. gingivalis;
(b) performing an antigen-antibody reaction or cross-reaction with the antigen of claim 18; and
(c) evaluating reaction results for diagnosis and/or prognosis assessment of infection caused by P. gingivalis or a chronic disease resulted from P. gingivalis infection.

29. A kit for diagnosis and prognosis of an infection caused by P. gingivalis, comprising
(a) a reagent comprising the antigen of claim 18, and
(b) an instruction on performing reaction using the reagent to detect specific antibodies from a biologic sample.

30. The kit of claim 29, wherein the biologic sample is a body fluid selected from the group consisting of blood, gingival crevicular fluid, urine, saliva, cerebrospinal fluid, pleural and ascites fluid, and amniotic fluid.

31. A method for treating a subject with an infection caused by P. gingivalis or a chronic disease resulted from P. gingivalis infection comprising administering to the subject an effective amount of the composition of claim 13.

32. A method for treating a subject with an infection caused by P. gingivalis or a chronic disease resulted from P. gingivalis infection comprising administering to the subject an effective amount of the composition of claim 14.


SEQUENCE LISTING (174 sequences)

SEQ ID NO: 1 - DNA, 1461 bp, synthetic construct
  source 1..1461: mol_type = other DNA
catatggaactagatagggatcccgaaggaaaggatttccaacaaccatacaccagcttcgtgcaaaccaaacagaaccgtgatggcctgtacgcgctgctgcgtaacaccgaaaacccgcgtatgcacttctaccaggagctgcaaagcgacatgtattgcaccaccatcaccgatggtaacagcctggcgccgtttgttaactgggacctgggtatcctgaacgatcacggccgtgcggacgaggatgaagtgagcggtattgcgggctactatttcgtttacaaccgtctgaaccagcaagcgaacgcgtttgtgaacaacaccgaagcggcgctgcagaaccaagtttataaaaacagcaccgaaatcgcgaacgcgaagagcttcctggcggagggcaaggttctgcaggcgctggcgatttggcgtctgatggaccgtttcagctttcacgagagcgtgaccgaagttaacagcggtgcgaaagatctgggcgtgatcctgctgaaggagtacaacccgggttatattggcccgcgtgcgaccaaagcgcaatgctacgactatatcctgagccgtctgagcgaggcgattgaagttctgccggagaaccgtgaaagcgtgctgtacgttagccgtgattacgcgtatgcgctgcgtgcgcgtatctacctggcgctgggtgaatatggtaaagcggcggcggatgcgaagatggtggttgataaatacccgctgattggtgcggcggatgcgagcgagttcgaaaacatttatcgtagcgatgcgaacaacccggagatcattttccgtggttttgcgagcgcgaccctgggcagctttaccgcgaccaccctgaacggtgcggcgccggcgggcaaggacatcaaatacaacccgagcgcggtgccgttccagtgggtggttgacctgtacgagaacgaagattttcgtaagagcgtgtatattgcgaaagtggttaagaaagacaagggttacctggttaacaagttcctggaagataaagcgtatcgtgacgtgcaggataagccgaacctgaaagttggcgcgcgttactttagcgtggcggaagtttatctgattctggtggagagcgcgctgcaaaccggtgataccccgaccgcggagaagtatctgaaagcgctgagcaaagcgcgtggcgcggaagttagcgtggttaacatggaggcgctgcaagcggagcgtacccgtgaactgatcggcgagggcagccgtctgcgtgacatggtgcgttggagcattccgaacaaccacgatgcgttcgaaacccagccgggtctggagggctttgcgaacaccaccccgctgaaggcgcaagcgccggtgggtttttacgcgtatacctgggaatttccgcaacgcgaccgtcaaaccaacccgcaactgattaagaactggccgatttaatgaaagctt

SEQ ID NO: 2 - protein, 482 aa, Porphyromonas gingivalis
MELDRDPEGKDFQQPYTSFVQTKQNRDGLYALLRNTENPRMHFYQELQSDMYCTTITDGNSLAPFVNWDLGILNDHGRADEDEVSGIAGYYFVYNRLNQQANAFVNNTEAALQNQVYKNSTEIANAKSFLAEGKVLQALAIWRLMDRFSFHESVTEVNSGAKDLGVILLKEYNPGYIGPRATKAQCYDYILSRLSEAIEVLPENRESVLYVSRDYAYALRARIYLALGEYGKAAADAKMVVDKYPLIGAADASEFENIYRSDANNPEIIFRGFASATLGSFTATTLNGAAPAGKDIKYNPSAVPFQWVVDLYENEDFRKSVYIAKVVKKDKGYLVNKFLEDKAYRDVQDKPNLKVGARYFSVAEVYLILVESALQTGDTPTAEKYLKALSKARGAEVSVVNMEALQAERTRELIGEGSRLRDMVRWSIPNNHDAFETQPGLEGFANTTPLKAQAPVGFYAYTWEFPQRDRQTNPQLIKNWPI

SEQ ID NO: 3 - DNA, 1449 bp, synthetic construct
  source 1..1449: mol_type = other DNA
catatgtgtgatctggatcgtaccccgcataatagcgacgtccagaaaccgtacgaagatatggcaaccaccgttcagtatcgcgacggtctgtattctgttctgcgcggcgcagaaaacgcaggtcgttataccctgtccgaatacatgagcgacatgtactgcgttatgcaaggcgacggcggtcacgcaaccccgtacgttacctggaccattccgcgtattgaaattgcggatcacgcaagtaactactacttcggcttcaaccgcctgattcagcaggcaaacgcatttgtaggcaacgttaaactggcgattgcgaacggcgtctacaaaaccgaagtcgacaaaaccaacgcgcagatttatctggcggaagcaaaaaccctgcaagcactggctctgtttcgtctgatggaacgctttgcgtatccgtacgatccgaacgaaaccaccagtccgaaaaacctgggcgttgttctgatcaaagaatacgatccgtgggcagttggcgcacgcgcaacccaaaccgaaacctatagctatatcatgagcctgctggacgaagcaattagcgttctgccggaaaccaacgctaacaacatgtatgtaagccgcgattacgctctgggtctgcgtgctcgcgttcacatggcgatggacaactatgcagaagcagcaaacgatattcgcgcgttctacaaaaaatacaacctgatcagcgcggcgaacagcgacgaatttgaagaagcctaccgcaaaatgagcagcaatccggaactgatctttcgcggttacgcgagcgtgaccaacggttacctggtttaccaggatctgatgggcgcaaccgcaagcggcaccaacgtcaaatataacccgcgcgttaccccgctgcagtgggtttgcgatctgtacgacgcagccgattatcgcaaaaaagtctacatcgtcgacaaagttaacggcgacggcggtaaaggctacgtagtcaacaaattcctgggcgatccggaactgcgcgaagacccgaaaaaagagaacttcaaaaccggctgccgctttttcagcctggctgaagcgtatctgattctggctgaagccgatatcatgaccggtaataccgctgaagcgatggaggtcctgaaagaactgagtaaaagtcgcggcgcagaagtttctggcgccgattacatgcagatcctgaaagacgagcgtacccgcgaaatgattggcgaaggttctcgtctgaacgatatgatccgctggaacatggatctggttgttagcccggttcaggcagttctgcataaaattgcggttccgaccattctgcaaaccgacgatccgacccgcgttccggcaggtttttacgcatttacctgggaaatcccgaatcgcgatctggttgttattccggaactggttcgtaattggccgaaacaataatga

SEQ ID NO: 4 - protein, 480 aa, Porphyromonas gingivalis
MCDLDRTPHNSDVQKPYEDMATTVQYRDGLYSVLRGAENAGRYTLSEYMSDMYCVMQGDGGHATPYVTWTIPRIEIADHASNYYFGFNRLIQQANAFVGNVKLAIANGVYKTEVDKTNAQIYLAEAKTLQALALFRLMERFAYPYDPNETTSPKNLGVVLIKEYDPWAVGARATQTETYSYIMSLLDEAISVLPETNANNMYVSRDYALGLRARVHMAMDNYAEAANDIRAFYKKYNLISAANSDEFEEAYRKMSSNPELIFRGYASVTNGYLVYQDLMGATASGTNVKYNPRVTPLQWVCDLYDAADYRKKVYIVDKVNGDGGKGYVVNKFLGDPELREDPKKENFKTGCRFFSLAEAYLILAEADIMTGNTAEAMEVLKELSKSRGAEVSGADYMQILKDERTREMIGEGSRLNDMIRWNMDLVVSPVQAVLHKIAVPTILQTDDPTRVPAGFYAFTWEIPNRDLVVIPELVRNWPKQ

SEQ ID NO: 5 - DNA, 1470 bp, synthetic construct
  source 1..1470: mol_type = other DNA
catatgctgaaccgcgatccggaagacaaagcgaaaaaagagccgttcaaaaccattgcgcaggcacgtcaaagtcgcgacggtctgtattctctgctgcgtgaaagcgaacaactggcatttcacgtctacgacgaggtccagtctgatatgtacaccaccaccaaaaacgacggcaatcagtactacccgtttgttgcttggcaactgggttctatggaaacccacggtcgcgcagacgaagatcaggtctacggcatcgcgggctattattttgaatacagccgcctgattcagcaggcaaatagctacatcgaggacatggagaaagcactggcgaacaacaccgacatgagcatcttcatcagcagcgaagaagttgaacgcgcaaaacgttatctggcggaagttcgcgttgttcaagcactggctaattggcgtctgatggatcgcttcgcgtataaatacgacgcggcaagcaatcagagtccgcagaatctgggcattgttctgatcaaagagttcaacccggcgtatattggtccgcgcgctacccaaaaagagtgctacgatcacattctgagcgcactggaagcagcaattaccgttctgccggagaaaaacccggacggtattacctatgcaggtcgcgattacgcacacgcactgcgcgcacgcgttcatctgtctatgggtaattatgcggacgcactggcagacgcaaaacagatcgtcgagaaatacccgctgatcaacgcgaacaacgcggaagatttcgcgaaaatctatcgtagcgacgcgaacaacccggaaattgtttttcgcgcatttgcttctccgaccaccggtgcagttggcgctacctctctgaacggtgcgagcgtcgtcaacaaaaagattaaatacgcgccgtttatcgttccgctgcagtgggttgttgatctgtacgaagacgccgattatcgcaaaagcgtctacatcgacaaaaccgtcagcaacggtagcgaaaaaggctacctggtcaacaaattcctggaggatccggcatatcgcgaaaccgcggatattccgattctgaaaatcggcgtccgcatgtttagcattgcggaagcctatctgatggttgctgagtgcgctcacatgaccggtgatgacgcaaccgcaattgcgtatctgaaaaaactgtctgcagcacgcggcggtaatattgataccggcgacgtcatgaaagcgatccaagaagaacgtacccgcgaactgattggcgaaggcgctcgtctgcgcgatatgattcgttggaacctgccgaacatcgacaaaaccgagattcaaccggcactgaccggttacgcacgcgaaattgttctggaacaaccggtaccggcaggtcattacgcatttacctgggaattcccgaaccgcgatcgtcaagttaatccgcatctgatcaaaaactggtaatgaaagctt

SEQ ID NO: 6 - protein, 485 aa, Porphyromonas gingivalis
MLNRDPEDKAKKEPFKTIAQARQSRDGLYSLLRESEQLAFHVYDEVQSDMYTTTKNDGNQYYPFVAWQLGSMETHGRADEDQVYGIAGYYFEYSRLIQQANSYIEDMEKALANNTDMSIFISSEEVERAKRYLAEVRVVQALANWRLMDRFAYKYDAASNQSPQNLGIVLIKEFNPAYIGPRATQKECYDHILSALEAAITVLPEKNPDGITYAGRDYAHALRARVHLSMGNYADALADAKQIVEKYPLINANNAEDFAKIYRSDANNPEIVFRAFASPTTGAVGATSLNGASVVNKKIKYAPFIVPLQWVVDLYEDADYRKSVYIDKTVSNGSEKGYLVNKFLEDPAYRETADIPILKIGVRMFSIAEAYLMVAECAHMTGDDATAIAYLKKLSAARGGNIDTGDVMKAIQEERTRELIGEGARLRDMIRWNLPNIDKTEIQPALTGYAREIVLEQPVPAGHYAFTWEFPNRDRQVNPHLIKNW

SEQ ID NO: 7 - DNA, 1455 bp, synthetic construct
  source 1..1455: mol_type = other DNA
aagctttcattagttcggccagtttttgatgatctgcgggttaacctgacgatcgcgaaccggaaattcccaggtaaatgcgtaatgacctgccggaaccggctgtttcagtttatccgctttcgccagaccaatctgcagaaataccggctggttctccatatcgtcgtggttattcggcaggttccagcgaatcatgtcgttcagacgactaccttcgccaatcagttcgcgggtacgttctgccataaccaggtccataatatcgccgtcttccagttgaccgccgcgtttttcgcacagggttttcagcagatcttttgcgcctgcagcatcaccagatttggccatgctctcggccataatcagataggcttccgcaacgctgaaaaagcggctggtgactttgaagtccggaacgttcggattgctctgatatgccgggttacccaggtatttaccaactacgtaacctttatcaacgccgcccccaatgacgtagaagtctttggtgatgtagcagcttttgcggtaatcggtatcgtcgtacagatcaactacccactgaaacggggcagaaatcggactactaacaaccagctgtgcaccagaatgccacagaaaaccagacagggtcgtttctgcaacggtaccaatggtaccagatgcaaacgcgcggaagacaatttccgggttgtttgcgtcgtcgcgataaacttcggcgaattttttcgcggtggtaacgtcaatcagcggatagttgtcgaccagaactttcgcgtcttcttttgccgcgtcgtaattgcccattgcaaagttaacgcgtgcgcgcagtgcgtgtgcataatgtttggaaacgcggatgttgccttcgttgcttttgttcggcagaacgtcaatcgcctggttcagacactccaggatgtagtcgtagcactctttctgggtattacgcggtgccgcaatcatcgggttgtagtctttgaccaggacaatacccagatcgtacggatgggtggttgctgccggatcgtatttgtacgcaaagcgttccatcagacgagacagagccagtgcttgcagggttttgccttccgcctggaagattttgacgtctttcagctctttctccaggtacaggttcagttccatgctacgttcgatgcgggtaacgaagtaatttgcgcgttgcagcaggctataatacgccagataatagctcgcgatttcgccgctactttccagtgcgcccagactccaggtaatgaaacgactactggtattaccgtcgttggtggtaaccgcgtacatatcagactgcagatcctgccagacaaaacgggtcggattttcggtactacgcagggttgcgtacagaccgtcgcgaaaatgtttggtctcgacgaagctcgcgaaatgatctttctgctcgtcagaaccgtccggatccatatg

SEQ ID NO: 8 - protein, 480 aa, Porphyromonas gingivalis
MDPDGSDEQKDHFASFVETKHFRDGLYATLRSTENPTRFVWQDLQSDMYAVTTNDGNTSSRFITWSLGALESSGEIASYYLAYYSLLQRANYFVTRIERSMELNLYLEKELKDVKIFQAEGKTLQALALSRLMERFAYKYDPAATTHPYDLGIVLVKDYNPMIAAPRNTQKECYDYILECLNQAIDVLPNKSNEGNIRVSKHYAHALRARVNFAMGNYDAAKEDAKVLVDNYPLIDVTTAKKFAEVYRDDANNPEIVFRAFASGTIGTVAETTLSGFLWHSGAQLVVSSPISAPFQWVVDLYDDTDYRKSCYITKDFYVIGGGVDKGYVVGKYLGNPAYQSNPNVPDFKVTSRFFSVAEAYLIMAESMAKSGDAAGAKDLLKTLCEKRGGQLEDGDIMDLVMAERTRELIGEGSRLNDMIRWNLPNNHDDMENQPVFLQIGLAKADKLKQPVPAGHYAFTWEFPVRDRQVNPQIIKNWPN

SEQ ID NO: 9 - DNA, 411 bp, synthetic construct
  source 1..411: mol_type = other DNA
catatgcaaaataggacagttaaagggactgtaatatcaagtgaagataatgaaccgctgattggcgcgaatgttgttgttgtgggtaacaccaccattggtgcggcgaccgacctggatggtaacttcaccctgagcgttccggcgaacgcgaagatgctgcgtgtgagctacagcggcatgaccaccaaagaggttgcgatcgcgaacgttatgaagattgtgctggacccggatagcaaagtgctggaacaggtggttgtgctgggttatggcaccggtcaaaagctgagcaccgttagcggtagcgttgcgaaggtgagcagcgagaaactggcggagaagccggtggcgaacattatggatgcgctgcaaggtcaagtggcgggtatgcaagtgtaatgaaagctt

SEQ ID NO: 10 - protein, 132 aa, Porphyromonas gingivalis
MQNRTVKGTVISSEDNEPLIGANVVVVGNTTIGAATDLDGNFTLSVPANAKMLRVSYSGMTTKEVAIANVMKIVLDPDSKVLEQVVVLGYGTGQKLSTVSGSVAKVSSEKLAEKPVANIMDALQGQVAGMQV

SEQ ID NO: 11 - protein, 25 aa, synthetic construct
ELDRDPEGKDFQQPYTSFVQTKQNR

SEQ ID NO: 12 - protein, 25 aa, synthetic construct
TKQNRDGLYALLRNTENPRMHFYQE

SEQ ID NO: 13 - protein, 25 aa, synthetic construct
HFYQELQSDMYCTTITDGNSLAPFV

SEQ ID NO: 14 - protein, 25 aa, synthetic construct
LAPFVNWDLGILNDHGRADEDEVSG

SEQ ID NO: 15 - protein, 25 aa, synthetic construct
DEVSGIAGYYFVYNRLNQQANAFVN

SEQ ID NO: 16 - protein, 25 aa, synthetic construct
NAFVNNTEAALQNQVYKNSTEIANA

SEQ ID NO: 17 - protein, 25 aa, synthetic construct
EIANAKSFLAEGKVLQALAIWRLMD

SEQ ID NO: 18 - protein, 25 aa, synthetic construct
WRLMDRFSFHESVTEVNSGAKDLGV

SEQ ID NO: 19 - protein, 25 aa, synthetic construct
KDLGVILLKEYNPGYIGPRATKAQC

SEQ ID NO: 20 - protein, 25 aa, synthetic construct
TKAQCYDYILSRLSEAIEVLPENRE

SEQ ID NO: 21 - protein, 25 aa, synthetic construct
PENRESVLYVSRDYAYALRARIYLA

SEQ ID NO: 22 - protein, 25 aa, synthetic construct
RIYLALGEYGKAAADAKMVVDKYPL

SEQ ID NO: 23 - protein, 25 aa, synthetic construct
DKYPLIGAADASEFENIYRSDANNP

SEQ ID NO: 24 - protein, 25 aa, synthetic construct
DANNPEIIFRGFASATLGSFTATTL

SEQ ID NO: 25 - protein, 25 aa, synthetic construct
TATTLNGAAPAGKDIKYNPSAVPFQ

SEQ ID NO: 26 - protein, 25 aa, synthetic construct
AVPFQWVVDLYENEDFRKSVYIAKV

SEQ ID NO: 27 - protein, 25 aa, synthetic construct
YIAKVVKKDKGYLVNKFLEDKAYRD

SEQ ID NO: 28 - protein, 25 aa, synthetic construct
KAYRDVQDKPNLKVGARYFSVAEVY

SEQ ID NO: 29 - protein, 25 aa, synthetic construct
VAEVYLILVESALQTGDTPTAEKYL

SEQ ID NO: 30 - protein, 25 aa, synthetic construct
AEKYLKALSKARGAEVSVVNMEALQ

SEQ ID NO: 31 - protein, 25 aa, synthetic construct
MEALQAERTRELIGEGSRLRDMVRW

SEQ ID NO: 32 - protein, 25 aa, synthetic construct
DMVRWSIPNNHDAFETQPGLEGFAN

SEQ ID NO: 33 - protein, 25 aa, synthetic construct
EGFANTTPLKAQAPVGFYAYTWEFP

SEQ ID NO: 34 - protein, 21 aa, synthetic construct
TWEFPQRDRQTNPQLIKNWPI

SEQ ID NO: 35 - protein, 25 aa, synthetic construct
CDLDRTPHNSDVQKPYEDMATTVQY

SEQ ID NO: 36 - protein, 25 aa, synthetic construct
TTVQYRDGLYSVLRGAENAGRYTLS

SEQ ID NO: 37 - protein, 25 aa, synthetic construct
RYTLSEYMSDMYCVMQGDGGHATPY

SEQ ID NO: 38 - protein, 25 aa, synthetic construct
HATPYVTWTIPRIEIADHASNYYFG

SEQ ID NO: 39 - protein, 25 aa, synthetic construct
NYYFGFNRLIQQANAFVGNVKLAIA

SEQ ID NO: 40 - protein, 25 aa, synthetic construct
KLAIANGVYKTEVDKTNAQIYLAEA

SEQ ID NO: 41 - protein, 25 aa, synthetic construct
YLAEAKTLQALALFRLMERFAYPYD

SEQ ID NO: 42 - protein, 25 aa, synthetic construct
AYPYDPNETTSPKNLGVVLIKEYDP

SEQ ID NO: 43 - protein, 25 aa, synthetic construct
KEYDPWAVGARATQTETYSYIMSLL

SEQ ID NO: 44 - protein, 25 aa, synthetic construct
IMSLLDEAISVLPETNANNMYVSRD

SEQ ID NO: 45 - protein, 25 aa, synthetic construct
YVSRDYALGLRARVHMAMDNYAEAA

SEQ ID NO: 46 - protein, 25 aa, synthetic construct
YAEAANDIRAFYKKYNLISAANSDE

SEQ ID NO: 47 - protein, 25 aa, synthetic construct
ANSDEFEEAYRKMSSNPELIFRGYA

SEQ ID NO: 48 - protein, 25 aa, synthetic construct
FRGYASVTNGYLVYQDLMGATASGT

SEQ ID NO: 49 - protein, 25 aa, synthetic construct
TASGTNVKYNPRVTPLQWVCDLYDA

SEQ ID NO: 50 - protein, 25 aa, synthetic construct
DLYDAADYRKKVYIVDKVNGDGGKG

SEQ ID NO: 51 - protein, 25 aa, synthetic construct
DGGKGYVVNKFLGDPELREDPKKEN

SEQ ID NO: 52 - protein, 25 aa, synthetic construct
PKKENFKTGCRFFSLAEAYLILAEA

SEQ ID NO: 53 - protein, 25 aa, synthetic construct
ILAEADIMTGNTAEAMEVLKELSKS

SEQ ID NO: 54 - protein, 25 aa, synthetic construct
ELSKSRGAEVSGADYMQILKDERTR

SEQ ID NO: 55 - protein, 25 aa, synthetic construct
DERTREMIGEGSRLNDMIRWNMDLV

SEQ ID NO: 56 - protein, 25 aa, synthetic construct
NMDLVVSPVQAVLHKIAVPTILQTD

SEQ ID NO: 57 - protein, 25 aa, synthetic construct
ILQTDDPTRVPAGFYAFTWEIPNRD

SEQ ID NO: 58 - protein, 19 aa, synthetic construct
IPNRDLVVIPELVRNWPKQ

SEQ ID NO: 59 - protein, 25 aa, synthetic construct
LNRDPEDKAKKEPFKTIAQARQSRD

SEQ ID NO: 60 - protein, 25 aa, synthetic construct
RQSRDGLYSLLRESEQLAFHVYDEV

SEQ ID NO: 61 - protein, 25 aa, synthetic construct
VYDEVQSDMYTTTKNDGNQYYPFVA

SEQ ID NO: 62 - protein, 25 aa, synthetic construct
YPFVAWQLGSMETHGRADEDQVYGI

SEQ ID NO: 63 - protein, 25 aa, synthetic construct
QVYGIAGYYFEYSRLIQQANSYIED

SEQ ID NO: 64 - protein, 25 aa, synthetic construct
SYIEDMEKALANNTDMSIFISSEEV

SEQ ID NO: 65 - protein, 25 aa, synthetic construct
SSEEVERAKRYLAEVRVVQALANWR

SEQ ID NO: 66 - protein, 25 aa, synthetic construct
LANWRLMDRFAYKYDAASNQSPQNL

SEQ ID NO: 67 - protein, 25 aa, synthetic construct
SPQNLGIVLIKEFNPAYIGPRATQK

SEQ ID NO: 68 - protein, 25 aa, synthetic construct
RATQKECYDHILSALEAAITVLPEK

SEQ ID NO: 69 - protein, 25 aa, synthetic construct
VLPEKNPDGITYAGRDYAHALRARV

SEQ ID NO: 70 - protein, 25 aa, synthetic construct
LRARVHLSMGNYADALADAKQIVEK

SEQ ID NO: 71 - protein, 25 aa, synthetic construct
QIVEKYPLINANNAEDFAKIYRSDA

SEQ ID NO: 72 - protein, 25 aa, synthetic construct
YRSDANNPEIVFRAFASPTTGAVGA

SEQ ID NO: 73 - protein, 25 aa, synthetic construct
GAVGATSLNGASVVNKKIKYAPFIV

SEQ ID NO: 74 - protein, 25 aa, synthetic construct
APFIVPLQWVVDLYEDADYRKSVYI

SEQ ID NO: 75 - protein, 25 aa, synthetic construct
KSVYIDKTVSNGSEKGYLVNKFLED

SEQ ID NO: 76 - protein, 25 aa, synthetic construct
KFLEDPAYRETADIPILKIGVRMFS

SEQ ID NO: 77 - protein, 25 aa, synthetic construct
VRMFSIAEAYLMVAECAHMTGDDAT

SEQ ID NO: 78 - protein, 25 aa, synthetic construct
GDDATAIAYLKKLSAARGGNIDTGD

SEQ ID NO: 79 - protein, 25 aa, synthetic construct
IDTGDVMKAIQEERTRELIGEGARL

SEQ ID NO: 80 - protein, 25 aa, synthetic construct
EGARLRDMIRWNLPNIDKTEIQPAL

SEQ ID NO: 81 - protein, 25 aa, synthetic construct
IQPALTGYAREIVLEQPVPAGHYAF

SEQ ID NO: 82 - protein, 24 aa, synthetic construct
GHYAFTWEFPNRDRQVNPHLIKNW

SEQ ID NO: 83 - protein, 25 aa, synthetic construct
MDPDGSDEQKDHFASFVETKHFRDG

SEQ ID NO: 84 - protein, 25 aa, synthetic construct
HFRDGLYATLRSTENPTRFVWQDLQ

SEQ ID NO: 85 - protein, 25 aa, synthetic construct
WQDLQSDMYAVTTNDGNTSSRFITW

SEQ ID NO: 86 - protein, 25 aa, synthetic construct
RFITWSLGALESSGEIASYYLAYYS

SEQ ID NO: 87 - protein, 25 aa, synthetic construct
LAYYSLLQRANYFVTRIERSMELNL

SEQ ID NO: 88 - protein, 25 aa, synthetic construct
MELNLYLEKELKDVKIFQAEGKTLQ

SEQ ID NO: 89 - protein, 25 aa, synthetic construct
GKTLQALALSRLMERFAYKYDPAAT

SEQ ID NO: 90 - protein, 25 aa, synthetic construct
DPAATTHPYDLGIVLVKDYNPMIAA

SEQ ID NO: 91 - protein, 25 aa, synthetic construct
PMIAAPRNTQKECYDYILECLNQAI

SEQ ID NO: 92 - protein, 25 aa, synthetic construct
LNQAIDVLPNKSNEGNIRVSKHYAH

SEQ ID NO: 93 - protein, 25 aa, synthetic construct
KHYAHALRARVNFAMGNYDAAKEDA

SEQ ID NO: 94 - protein, 25 aa, synthetic construct
AKEDAKVLVDNYPLIDVTTAKKFAE

SEQ ID NO: 95 - protein, 25 aa, synthetic construct
KKFAEVYRDDANNPEIVFRAFASGT

SEQ ID NO: 96 - protein, 25 aa, synthetic construct
FASGTIGTVAETTLSGFLWHSGAQL

SEQ ID NO: 97 - protein, 25 aa, synthetic construct
SGAQLVVSSPISAPFQWVVDLYDDT

SEQ ID NO: 98 - protein, 25 aa, synthetic construct
LYDDTDYRKSCYITKDFYVIGGGVD

SEQ ID NO: 99 - protein, 25 aa, synthetic construct
GGGVDKGYVVGKYLGNPAYQSNPNV

SEQ ID NO: 100 - protein, 25 aa, synthetic construct
SNPNVPDFKVTSRFFSVAEAYLIMA

SEQ ID NO: 101 - protein, 25 aa, synthetic construct
YLIMAESMAKSGDAAGAKDLLKTLC

SEQ ID NO: 102 - protein, 25 aa, synthetic construct
LKTLCEKRGGQLEDGDIMDLVMAER

SEQ ID NO: 103 - protein, 25 aa, synthetic construct
VMAERTRELIGEGSRLNDMIRWNLP

SEQ ID NO: 104 - protein, 25 aa, synthetic construct
RWNLPNNHDDMENQPVFLQIGLAKA

SEQ ID NO: 105 - protein, 25 aa, synthetic construct
GLAKADKLKQPVPAGHYAFTWEFPV

SEQ ID NO: 106 - protein, 20 aa, synthetic construct
WEFPVRDRQVNPQIIKNWPN

SEQ ID NO: 107 - protein, 18 aa, synthetic construct
QNRTVKGTVISSEDNEPL

SEQ ID NO: 108 - protein, 18 aa, synthetic construct
DNEPLIGANVVVVGNTTI

SEQ ID NO: 109 - protein, 18 aa, synthetic construct
GNTTIGAATDLDGNFTLS

SEQ ID NO: 110 - protein, 18 aa, synthetic construct
NFTLSVPANAKMLRVSYS

SEQ ID NO: 111 - protein, 18 aa, synthetic construct
RVSYSGMTTKEVAIANVM

SEQ ID NO: 112 - protein, 18 aa, synthetic construct
IANVMKIVLDPDSKVLEQ

SEQ ID NO: 113 - protein, 18 aa, synthetic construct
KVLEQVVVLGYGTGQKLS

SEQ ID NO: 114 - protein, 18 aa, synthetic construct
GQKLSTVSGSVAKVSSEK

SEQ ID NO: 115 - protein, 18 aa, synthetic construct
VSSEKLAEKPVANIMDAL

SEQ ID NO: 116 - protein, 14 aa, synthetic construct
IMDALQGQVAGMQV

SEQ ID NO: 117 - protein, 25 aa, synthetic construct
KGTVISSEDNEPLIGANVVVVGNTT

SEQ ID NO: 118 - protein, 25 aa, synthetic construct
VGNTTIGAATDLDGNFTLSVPANAK

SEQ ID NO: 119 - protein, 25 aa, synthetic construct
PANAKMLRVSYSGMTTKEVAIANVM

SEQ ID NO: 120 - protein, 25 aa, synthetic construct
IANVMKIVLDPDSKVLEQVVVLGYG

SEQ ID NO: 121 - protein, 25 aa, synthetic construct
VLGYGTGQKLSTVSGSVAKVSSEKL

SEQ ID NO: 122 - protein, 25 aa, synthetic construct
SSEKLAEKPVANIMDALQGQVAGMQ

SEQ ID NO: 123 - protein, 45 aa, synthetic construct
YIAKVVKKDKGYLVNKFLEDKAYRDVQDKPNLKVGARYFSVAEVY

SEQ ID NO: 124 - protein, 45 aa, synthetic construct
KSVYIDKTVSNGSEKGYLVNKFLEDPAYRETADIPILKIGVRMFS

SEQ ID NO: 125 - protein, 45 aa, synthetic construct
MEALQAERTRELIGEGSRLRDMVRWSIPNNHDAFETQPGLEGFAN

SEQ ID NO: 126 - protein, 45 aa, synthetic construct
VMAERTRELIGEGSRLNDMIRWNLPNNHDDMENQPVFLQIGLAKA

SEQ ID NO: 127 - protein, 75 aa, synthetic construct
QLEDGDIMDLVMAERTRELIGEGSRLNDMIRWNLPNNHDDMENQPVFLQIGLAKADKLKQPVPAGHYAFTWEFPV

SEQ ID NO: 128 - DNA, 357 bp, Porphyromonas gingivalis
  source 1..357: mol_type = other DNA
gaggttcagctgcagcagtctggggcagagcttgtgaagccaggggcctcagtcaagttgtcctgcacagcttctggcttcaacattaaagacacctatatacactggatgagacagaggcctgaacagggcctggagtggattggaaggattgatcctgcgaatggtaatattaaatctgacccgaaattccaggtcaaggccactatcaccgcagacacatcctccaacacagcctacctgcaggtcagcagcctgacatctgacgacactgccgtctattactgtgctagatgggtctacggtaatagcccctttgactactggggccaaggcaccactctcacagtctcctca

SEQ ID NO: 129 - protein, 119 aa, Porphyromonas gingivalis
EVQLQQSGAELVKPGASVKLSCTASGFNIKDTYIHWMRQRPEQGLEWIGRIDPANGNIKSDPKFQVKATITADTSSNTAYLQVSSLTSDDTAVYYCARWVYGNSPFDYWGQGTTLTVSS

SEQ ID NO: 130 - protein, 5 aa, Porphyromonas gingivalis
DTYIH

SEQ ID NO: 131 - protein, 17 aa, Porphyromonas gingivalis
RIDPANGNIKSDPKFQV

SEQ ID NO: 132 - protein, 10 aa, Porphyromonas gingivalis
WVYGNSPFDY

SEQ ID NO: 133 - DNA, 282 bp, Porphyromonas gingivalis
  source 1..282: mol_type = other DNA
gacattgtgatgacacagtctccatcctccctgagtgtgtcagcaggagagaaggtcactatgagctgctggtccagtcagagtctgttaatcaactgttgttctgcggggcatccactagggaatctggggtccctgatcgcttcacaggcagtggatctggaaccgatttcactctttccatcagcagtgtgcaggccgaagacctggcagtttattactgtcagaatgatcatagttgtcctattcacgttcggctcggggacaaagttggaaataaaa

SEQ ID NO: 134 - protein, 94 aa, Porphyromonas gingivalis
DIVMTQSPSSLSVSAGEKVTMSCWSSQSLLINCCSAGHPLGNLGSLIASQAVDLEPISLFPSAVCRPKTWQFITVRMIIVVLFTFGSGTKLEIK

SEQ ID NO: 135 - protein, 11 aa, Porphyromonas gingivalis
WSSQSLLINCC

SEQ ID NO: 136 - protein, 7 aa, Porphyromonas gingivalis
QAVDLEP

SEQ ID NO: 137 - protein, 5 aa, Porphyromonas gingivalis
KLEIK

SEQ ID NO: 138 - DNA, 360 bp, Porphyromonas gingivalis
  source 1..360: mol_type = other DNA
gaggttcagctgcagcagtctggggcagagcttgtgaagccaggggcctcagtcaagttgtcctgcacagcttcaggcttcaacattaaagacacctatatgcactggatgaaacagaggcctgaacagggcctggagtggattggaaggattgatcctgcgaatggtgatactaaatatgacccgaagttccagggcaaggccactataacagcagacacatcctccaacacagcctacctgcagctcagcagcctgacatctgaggacactgccgtctattactgtgctagagaggatggtacctccccctatgctatgaactactggggtcaaggaacctcagtcaccgtctcctca

SEQ ID NO: 139 - protein, 120 aa, Porphyromonas gingivalis
EVQLQQSGAELVKPGASVKLSCTASGFNIKDTYMHWMKQRPEQGLEWIGRIDPANGDTKYDPKFQGKATITADTSSNTAYLQLSSLTSEDTAVYYCAREDGTSPYAMNYWGQGTSVTVSS

SEQ ID NO: 140 - protein, 5 aa, Porphyromonas gingivalis
DTYMH

SEQ ID NO: 141 - protein, 17 aa, Porphyromonas gingivalis
RIDPANGDTKYDPKFQG

SEQ ID NO: 142 - protein, 11 aa, Porphyromonas gingivalis
EDGTSPYAMNY

SEQ ID NO: 143 - DNA, 336 bp, Porphyromonas gingivalis
  source 1..336: mol_type = other DNA
gatgttttgatgacccaaactccactctccctgcctgtcagtcttggagatcaagcctccatctcttgcagatctagtcagaacattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttctaggttcacatgttcctccgacgttcggtggaggcaccaagctgaaaatcaaa

SEQ ID NO: 144 - protein, 112 aa, Porphyromonas gingivalis
VLMTQTPLSLPVSLGDQASISCRSSQNIVHSNGNTYLEWYLQKPGQSPKLLIYKVSNRFSGVPDRFSGSGSGTDFTLKISRVEAEDLGVYYCFLGSHVPPTFGGGTKLKIKI

SEQ ID NO: 145 - protein, 16 aa, Porphyromonas gingivalis
RSSQNIVHSNGNTYLE

SEQ ID NO: 146 - protein, 7 aa, Porphyromonas gingivalis
KVSNRFS

SEQ ID NO: 147 - protein, 9 aa, Porphyromonas gingivalis
FLGSHVPPT

SEQ ID NO: 148 - DNA, 375 bp, Porphyromonas gingivalis
  source 1..375: mol_type = other DNA
aaggtccagttgcagcagtctggaactgggctggtgaaacccggggcatcagtgaagctgtcctgcaaggcttctggctacattttcactgaatatattatacattgggtaaagcagaggtctggacagggtcttgagtggattgggtggttttaccctggaagtgggagtataaagtacaatgagaaattcaaggacaaggccacaataactgcggacaaatcctccagcacagtctatatggagctgagtagactgacatctgaggactctgcggtctatttttgtgcaagacacgaagacctccacgataggtccggcggaaggtcctggtttgcttactggggccaagggactccggtcactgtctctgca

SEQ ID NO: 149 - protein, 125 aa, Porphyromonas gingivalis
KVQLQQSGTGLVKPGASVKLSCKASGYIFTEYIIHWVKQRSGQGLEWIGWFYPGSGSIKYNEKFKDKATITADKSSSTVYMELSRLTSEDSAVYFCARHEDLHDRSGGRSWFAYWGQGTPVTVSA

SEQ ID NO: 150 - protein, 5 aa, Porphyromonas gingivalis
EYIIH

SEQ ID NO: 151 - protein, 17 aa, Porphyromonas gingivalis
WFYPGSGSIKYNEKFKD

SEQ ID NO: 152 - protein, 16 aa, Porphyromonas gingivalis
HEDLHDRSGGRSWFAY

SEQ ID NO: 153 - DNA, 336 bp, Porphyromonas gingivalis
  source 1..336: mol_type = other DNA
gatgttttgatgacccaaactccactctccctgcctgtcagtcttggagatcaagtctccatgtcttgcagatctagtcagaccattgtacatactaatggaaaaacctatttagaatggtacctgcagagaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 154 - protein, 112 aa, Porphyromonas gingivalis
DVLMTQTPLSLPVSLGDQVSMSCRSSQTIVHTNGKTYLEWYLQRPGQSPKLLIYKVSNRFSGVPDRFSGSGSGTDFTLKISRVEAEDLGVYYCFQGSHVPLTFGAGTKLELK

SEQ ID NO: 155 - protein, 16 aa, Porphyromonas gingivalis
RSSQTIVHTNGKTYLE

SEQ ID NO: 156 - protein, 7 aa, Porphyromonas gingivalis
KVSNRFS

SEQ ID NO: 157 - protein, 9 aa, Porphyromonas gingivalis
FQGSHVPLT

SEQ ID NO: 158 - DNA, 357 bp, Porphyromonas gingivalis
  source 1..357: mol_type = other DNA
gaggtgaagctggtggagtctggaggaggcttggtacagcctgggggttctctgagactctcctgtgcaacttctgggttcaccttcactgattactacatgagctgggtccgccagcctccaggaaaggcacttgagtggttggcttttattagagacagaactaatggttacacaacagagtacagtgcatctgtgaggggtcggttcaccatctccagagataattcccaaagcatcctctatcttcacatgaacaccctgagagctgaggacagtgccacttattactgttcaagagataggagtggcccgtttgcttactggggccaaggtactctggtcactgtctctgca

SEQ ID NO: 159 - protein, 119 aa, Porphyromonas gingivalis
EVKLVESGGGLVQPGGSLRLSCATSGFTFTDYYMSWVRQPPGKALEWLAFIRDRTNGYTTEYSASVRGRFTISRDNSQSILYLHMNTLRAEDSATYYCSRDRSGPFAYWGQGTLVTVSA

SEQ ID NO: 160 - protein, 5 aa, Porphyromonas gingivalis
DYYMS

SEQ ID NO: 161 - protein, 19 aa, Porphyromonas gingivalis
FIRDRTNGYTTEYSASVRG

SEQ ID NO: 162 - protein, 8 aa, Porphyromonas gingivalis
DRSGPFAY

SEQ ID NO: 163 - DNA, 321 bp, Porphyromonas gingivalis
  source 1..321: mol_type = other DNA
gacattgtgatgaccccttctcaaaaattcatgtccacatcagtaggagacagggtcagcgtcacctgcaaggccagtcagaatgtgggtactaatgtagtctggtatcaacagaaaccaggacaatctcctaaagcattgatttactcggcatccaaccggtacagtggagtccctgatcgcttcacaggcagtggatctgggacagatttcactctcaccatcagcaatgtgcagtctgaagacttggcagagtatttctgtcagcaatataacagttatcctctcacgttcggaggggggaccaagctggaaataaga

SEQ ID NO: 164 - protein, 107 aa, Porphyromonas gingivalis
DIVMTPSQKFMSTSVGDRVSVTCKASQNVGTNVVWYQQKPGQSPKALIYSASNRYSGVPDRFTGSGSGTDFTLTISNVQSEDLAEYFCQQYNSYPLTFGGGTKLEIR

SEQ ID NO: 165 - protein, 11 aa, Porphyromonas gingivalis
KASQNVGTNVV

SEQ ID NO: 166 - protein, 7 aa, Porphyromonas gingivalis
SASNRYS

SEQ ID NO: 167 - protein, 9 aa, Porphyromonas gingivalis
QQYNSYPLT

SEQ ID NO: 168 - DNA, 2232 bp, synthetic construct
  source 1..2232: mol_type = other DNA
gcggccgcaaactacaagacagacttgcaaaagaaggcatgcacagctcagcactgctctgttgcctggtcctcctgactggggtgagggccgaggtgaagctggtggagagcggaggggggctggtgcagccaggaggaagcctgagactgagctgtgccacaagcgggttcacatttaccgactattatatgagctgggtgaggcagccacctggcaaggccctggagtggctggcttttattagagacaggacaaacggatacaccaccgagtacagcgccagcgtgagaggcagatttaccatcagccgggacaacagccagagcatcctgtacctgcacatgaacaccctgagagccgaagattctgctacatactactgcagcagagacagaagcggacccttcgcttactggggccagggaaccctggtgaccgtgtccgccgctaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccgcaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaaggaggaggaggaagcggaggaggagggagcggaggagggggaagcaaggtgcagctgcagcagagcggaacaggactggtgaagccaggagccagcgtgaagctgagctgcaaggccagtggatacatcttcacagagtacattatccactgggtgaagcagagaagtggccagggactggagtggatcggatggttttaccccggctccggcagcatcaaatacaacgaaaaattcaaggacaaggctacaattaccgccgacaaaagcagtagcaccgtgtacatggagctgagcagactgaccagcgaggacagcgccgtgtacttttgcgccagacacgaggatctgcacgacagaagcggaggccgcagctggttcgcctactggggacagggaacccccgtgaccgtgagcgccggaggaggaggcagcggaggaggcggaagcggaggcggaggaagcgacgtgctgatgacccagacacccctgagcctgccagtgagtctgggcgatcaggtgtctatgagttgcagaagcagccagacaattgtgcacacaaacggaaagacatacctggagtggtacctgcagaggcctggccagagccccaagctgctgatctacaaggtgagcaacaggttcagcggagtgcccgacagattcagcggatcaggcagcggaaccgacttcaccctgaagatctccagagtggaagccgaggacctgggcgtgtactactgcttccagggaagccacgtgccactgacctttggcgccgggaccaagctggagctgaagtgattctaga

SEQ ID NO: 169 - DNA, 357 bp, synthetic construct
  source 1..357: mol_type = other DNA
gaggtgaagctggtggagagcggaggggggctggtgcagccaggaggaagcctgagactgagctgtgccacaagcgggttcacatttaccgactattatatgagctgggtgaggcagccacctggcaaggccctggagtggctggcttttattagagacaggacaaacggatacaccaccgagtacagcgccagcgtgagaggcagatttaccatcagccgggacaacagccagagcatcctgtacctgcacatgaacaccctgagagccgaagattctgctacatactactgcagcagagacagaagcggacccttcgcttactggggccagggaaccctggtgaccgtgtccgcc

SEQ ID NO: 170 - DNA, 375 bp, synthetic construct
  source 1..375: mol_type = other DNA
aaggtgcagctgcagcagagcggaacaggactggtgaagccaggagccagcgtgaagctgagctgcaaggccagtggatacatcttcacagagtacattatccactgggtgaagcagagaagtggccagggactggagtggatcggatggttttaccccggctccggcagcatcaaatacaacgaaaaattcaaggacaaggctacaattaccgccgacaaaagcagtagcaccgtgtacatggagctgagcagactgaccagcgaggacagcgccgtgtacttttgcgccagacacgaggatctgcacgacagaagcggaggccgcagctggttcgcctactggggacagggaacccccgtgaccgtgagcgcc

SEQ ID NO: 171 - DNA, 336 bp, synthetic construct
  source 1..336: mol_type = other DNA
gacgtgctgatgacccagacacccctgagcctgccagtgagtctgggcgatcaggtgtctatgagttgcagaagcagccagacaattgtgcacacaaacggaaagacatacctggagtggtacctgcagaggcctggccagagccccaagctgctgatctacaaggtgagcaacaggttcagcggagtgcccgacagattcagcggatcaggcagcggaaccgacttcaccctgaagatctccagagtggaagccgaggacctgggcgtgtactactgcttccagggaagccacgtgccactgacctttggcgccgggaccaagctggagctgaag

SEQ ID NO: 172 - protein, 728 aa, Porphyromonas gingivalis
MHSSALLCCLVLLTGVRAEVKLVESGGGLVQPGGSLRLSCATSGFTFTDYYMSWVRQPPGKALEWLAFIRDRTNGYTTEYSASVRGRFTISRDNSQSILYLHMNTLRAEDSATYYCSRDRSGPFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGGGGSGGGGSGGGGSKVQLQQSGTGLVKPGASVKLSCKASGYIFTEYIIHWVKQRSGQGLEWIGWFYPGSGSIKYNEKFKDKATITADKSSSTVYMELSRLTSEDSAVYFCARHEDLHDRSGGRSWFAYWGQGTPVTVSAGGGGSGGGGSGGGGSDVLMTQTPLSLPVSLGDQVSMSCRSSQTIVHTNGKTYLEWYLQRPGQSPKLLIYKVSNRFSGVPDRFSGSGSGTDFTLKISRVEAEDLGVYYCFQGSHVPLTFGAGTKLELK

SEQ ID NO: 173 - DNA, 744 bp, synthetic construct
  source 1..744: mol_type = other DNA
gcggccgcaaactacaagacagacttgcaaaagaaggcatgcacagctcagcactgctctgttgcctggtcctcctgactggggtgagggccgacattgtgatgacccccagccagaaatttatgagcacctccgtgggcgacagagtgagcgtgacctgcaaggcctctcagaacgtgggcaccaacgtggtgtggtaccagcagaaaccaggccagagccccaaagccctgatctacagcgccagcaacagatacagcggagtgcctgatagattcaccggcagcggcagcggcaccgacttcaccctgactatcagcaacgtgcagtcagaggacctggccgagtacttctgccagcagtacaacagctacccactgaccttcgggggcggcaccaagctggagatcagaagagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgattctaga

SEQ ID NO: 174 - protein, 232 aa, Porphyromonas gingivalis
MHSSALLCCLVLLTGVRADIVMTPSQKFMSTSVGDRVSVTCKASQNVGTNVVWYQQKPGQSPKALIYSASNRYSGVPDRFTGSGSGTDFTLTISNVQSEDLAEYFCQQYNSYPLTFGGGTKLEIRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC